(19) United States
(12) Patent Application Publication
(10) Pub. No.: US 2025/0369013 A1
(43) Pub. Date: Dec. 4, 2025

(54) ADENOVIRAL HELPER VECTORS

(71) Applicant: Ensoma, Inc., Boston, MA (US)

(72) Inventor: Soumitra Roy, Townsend, DE (US)

(21) Appl. No.: 18/877,814

(22) PCT Filed: Jun. 28, 2023
(86) PCT No.: PCT/US2023/026399

§ 371 Date: Dec. 20, 2024

Related U.S. Application Data
(60) Provisional application No. 63/356,821, filed on Jun. 29, 2022.

(51) Int. Cl. C12N 15/86; C12N 7/00

(52) U.S. Cl. CPC C12N 15/86; C12N 7/00; C12N 2710/10321; C12N 2710/10343; C12N 2710/10352; C12N 2800/30

(57) ABSTRACT

The present disclosure provides, among other things, helper genomes and vectors useful in gene therapy, e.g., for production of helper-dependent donor vectors. Helper genomes of the present disclosure include a conditionally defective packaging sequence.

### PRIORITY APPLICATION

The present application claims the benefit of U.S. Provisional Patent Application No. 63/356,821, filed Jun. 29, 2022, the content of which is hereby incorporated by reference herein in its entirety.

### BACKGROUND

Many medical conditions are caused by genetic mutation and/or are treatable, at least in part, by gene therapy. Some conditions are particularly treatable by modification of target cells such as hematopoietic stem cells (HSCs). Compositions and methods for gene therapy are therefore needed.

### SUMMARY

Gene therapy can treat many conditions that have a genetic component, including without limitation hemoglobinopathies, immune deficiencies, and cancers. In various gene therapies, hematopoietic stem cells (HSCs) are an important target. However, current methods and compositions for gene therapy, and particularly for modifying HSCs, are limited. For instance, some vectors for gene therapy such as lentiviral vectors have a relatively limited payload capacity. Others, such as adenoviral serotype 5 (Ad5) vectors, are characterized by substantial payload capacity but are sufficiently prevalent such that the majority of humans have antibodies directed against proteins of such vectors, some of which antibodies may be neutralizing. The present disclosure provides, among other things, adenoviral helper genomes and vectors useful in gene therapy, e.g., for production of helper-dependent adenoviral donor vectors.

The present disclosure includes, among other things, Ad3, Ad7, Ad11, Ad14, Ad16, Ad21, Ad34, or Ad50 serotype helper vectors and Ad3, Ad7, Ad11, Ad14, Ad16, Ad21, Ad34, or Ad50 serotype helper genomes (e.g., “recombinant” or “engineered” adenoviral vectors and genomes). Adenoviral helper-dependent vectors are a type of vector that can be particularly useful for viral gene therapy, e.g., where the vector includes a donor genome that encodes a therapeutic payload for delivery to a recipient. Donor genomes of adenoviral helper-dependent vectors are engineered to remove viral coding sequences that are required for viral propagation and/or contribute to viral propagation, such that the helper-dependent vectors are deficient for propagation in recipients (e.g., human recipients receiving gene therapy including the helper-dependent vector). Because adenoviral helper-dependent donor genomes do not encode proteins used in viral production, they are dependent on other sources of viral proteins (e.g., expression from an adenoviral “helper” genome of the same serotype). For example, for packaging into vector, helper-dependent adenoviral genomes can be delivered to a cell that includes a nucleic acid sequence that provides viral proteins in trans. Viral proteins can be provided by an adenoviral helper genome engineered to reduce or eliminate packaging of the helper genome into helper-dependent donor vectors. Packaging of adenoviral helper genome into adenoviral donor vectors risks propagation in the recipient.

Adenoviral helper vectors must be conditionally competent (i.e., conditionally deficient or conditionally defective) for propagation. One means of achieving conditional propagation deficiency is by engineering of a conditionally defective packaging sequence in the helper genome (e.g., a packaging sequence that can mediate packaging of the helper genome, or mediate packaging of the helper genome more efficiently, in a first state or condition as compared to a second state or condition). The present disclosure includes, among other things, adenoviral helper genomes that include two recombinase sites positioned such that the two recombinase sites flank a packaging sequence, where the two recombinase sites are sites for the same recombinase. Positions of such recombinase sites to produce a conditionally defective packaging sequence in an adenoviral helper vector cannot be predicted from existing knowledge relating to other vectors. To the contrary, relevant sequences of Ad3, Ad7, Ad11, Ad14, Ad16, Ad21, Ad34, and Ad50 serotype genomes are very different from, e.g., corresponding sequences of Ad5 (compare, e.g., the 5′ 600 to 620 nucleotides of Ad3, Ad7, Ad11, Ad14, Ad16, Ad21, Ad34, or Ad50 and Ad5). Moreover, packaging sequences are serotype-specific. The Ad3, Ad7, Ad11, Ad14, Ad16, Ad21, Ad34, and Ad50 packaging sequence includes sequences that correspond to at least Ad5 packaging signal sequences AI, AII, AIII, AIV, and AV, but are unique to Ad3, Ad7, Ad11, Ad14, Ad16, Ad21, Ad34, and Ad50. Accordingly, production of an adenoviral helper vector requires several unpredictable determinations, including (1) identification of the adenoviral packaging sequence to be flanked by recombinase sites (e.g., loxP sites) by inserting or positioning recombinase sites in the subject genome, which is not straightforward where sequence similarity is limited; (2) identification of recombinase site insertions or positions that do not negate propagation of the helper vector (under conditions where the flanked packaging sequence is not excised), which cannot be predicted; and/or (3) identification of spacing between the recombinase sites that permits efficient deletion of the packaging sequence while reducing helper virus packaging during production of helper-dependent adenoviral donor vectors (e.g., in a cre recombinase-expressing cell line such as the 116 cell line). Thus, the present disclosure includes placement of recombinase sites (e.g., loxP recombinase sites) flanking adenoviral packaging sequences to produce conditionally defective packaging sequences in Ad3, Ad7, Ad11, Ad14, Ad16, Ad21, Ad34, and Ad50 helper genomes. In various embodiments, presence of the conditionally defective packaging sequence in an adenoviral helper genome renders the adenoviral helper genome conditionally defective for propagation, in that excision of the flanked adenoviral packaging sequence by recombination of the recombinase sites renders the adenoviral helper genome defective for packaging.

The present disclosure further includes the recognition that, in various embodiments, packaging sequence inversion can reduce the likelihood of mutations that bypass or disrupt conditionality of propagation and/or packaging. One problem that has characterized various donor vector production systems is that, when a helper genome is present in the same cell or system as a donor genome that includes a wild type or reference packaging sequence, all or a portion of a conditionally defective packaging sequence, or a genome fragment including the same, can be exchanged by homologous recombination with the donor genome for a corresponding fragment of the donor genome that includes the wild type or reference packaging sequence (which can be referred to herein as packaging sequence recombination). When packaging sequence recombination causes a modification of the helper genome that removes at least one of the recombinase sites flanking a packaging sequence of a conditionally defective packaging sequence, the event can be referred to as recombinase site-excising homologous recombination. When recombinase site-excising homologous recombination occurs, conditionality is lost. As a result, helper genomes can be packaged into vectors in the same manner as donor genomes (even in the presence of recombinases that would otherwise render the helper genome defective for packaging), and the production of donor vectors can be contaminated by production of vectors that include helper genomes.

Packaging sequence inversion as provided herein can reduce and/or eliminate recombinase site-excising homologous recombination at least in part by reducing overall homology between helper and donor genomes for any single strand orientation (particularly in packaging sequences and genome fragments including packaging sequences), thereby reducing the potential for packaging sequence recombination. While the present disclosure includes discussion of Ad3, Ad7, Ad11, Ad14, Ad16, Ad21, Ad34, and Ad50 vectors in particular, those of skill in the art will appreciate that packaging sequence inversion will be beneficial for helper genomes of diverse adenoviral serotypes and diverse types of viral vectors.

In at least one aspect, the present disclosure provides a recombinant adenoviral helper genome including: a 5′ inverted terminal repeat (ITR); a 3′ ITR; and a packaging sequence; where the 5′ ITR, the 3′ ITR, and the packaging sequence are each derived from a species B adenovirus of a serotype selected from Ad3, Ad7, Ad11, Ad14, Ad16, Ad21, Ad34, or Ad50; where the packaging sequence is flanked by or includes recombinase direct repeats including a first recombinase direct repeat and a second recombinase direct repeat; where the position of the first recombinase direct repeat corresponds to a position that is within 10 nucleotides of an L1, L2, L3, or L4 site of a reference sequence for the serotype of the packaging sequence, as set forth in Table 21; and where the position of the second recombinase direct repeat corresponds to a position that is within 10 nucleotides of an R1, R2, or R3 site of a reference sequence for the serotype of the packaging sequence, as set forth in Table 21. In various embodiments, the position of the first recombinase direct repeat corresponds to a position that is within 10 nucleotides of an L1 site of a reference sequence for the serotype of the packaging sequence, as set forth in Table 21; and the position of the second recombinase direct repeat corresponds to a position that is within 10 nucleotides of an R3 site of a reference sequence for the serotype of the packaging sequence, as set forth in Table 21. In various embodiments, the position of the first recombinase direct repeat corresponds to a position that is within 10 nucleotides of an L2 site of a reference sequence for the serotype of the packaging sequence, as set forth in Table 21; and the position of the second recombinase direct repeat corresponds to a position that is within 10 nucleotides of an R1 site of a reference sequence for the serotype of the packaging sequence, as set forth in Table 21. In various embodiments, the position of the first recombinase direct repeat corresponds to a position that is within 10 nucleotides of an L3 site of a reference sequence for the serotype of the packaging sequence, as set forth in Table 21; and the position of the second recombinase direct repeat corresponds to a position that is within 10 nucleotides of an R2 site of a reference sequence for the serotype of the packaging sequence, as set forth in Table 21. In various embodiments, the position of the first recombinase direct repeat corresponds to a position that is within 10 nucleotides of an L4 site of a reference sequence for the serotype of the packaging sequence, as set forth in Table 21; and the position of the second recombinase direct repeat corresponds to a position that is within 10 nucleotides of an R1 site of a reference sequence for the serotype of the packaging sequence, as set forth in Table 21.

In various embodiments, the position of the first recombinase direct repeat corresponds to a position at an L1, L2, L3, or L4 site of a reference sequence for the serotype of the packaging sequence, as set forth in Table 21; and where the position of the second recombinase direct repeat corresponds to a position that is at an R1, R2, or R3 site of a reference sequence for the serotype of the packaging sequence, as set forth in Table 21. In various embodiments, the position of the first recombinase direct repeat corresponds to a position at an L1 site of a reference sequence for the serotype of the packaging sequence, as set forth in Table 21; and the position of the second recombinase direct repeat corresponds to a position that is at an R3 site of a reference sequence for the serotype of the packaging sequence, as set forth in Table 21. In various embodiments, the position of the first recombinase direct repeat corresponds to a position at an L2 site of a reference sequence for the serotype of the packaging sequence, as set forth in Table 21; and the position of the second recombinase direct repeat corresponds to a position that is at an R1 site of a reference sequence for the serotype of the packaging sequence, as set forth in Table 21. In various embodiments, the position of the first recombinase direct repeat corresponds to a position at an L3 site of a reference sequence for the serotype of the packaging sequence, as set forth in Table 21; and the position of the second recombinase direct repeat corresponds to a position that is at an R2 site of a reference sequence for the serotype of the packaging sequence, as set forth in Table 21. In various embodiments, the position of the first recombinase direct repeat corresponds to a position at an L4 site of a reference sequence for the serotype of the packaging sequence, as set forth in Table 21; and the position of the second recombinase direct repeat corresponds to a position that is at an R1 site of a reference sequence for the serotype of the packaging sequence, as set forth in Table 21.

In various embodiments, the position of the first recombinase direct repeat corresponds to a position at an L1, L2, L3, or L4 site of a reference sequence for the serotype of the packaging sequence, as set forth in any one of Tables 22-27; and the position of the second recombinase direct repeat corresponds to a position that is at an R1, R2, or R3 site of a reference sequence for the serotype of the packaging sequence, as set forth in any one of Tables 22-27. In various embodiments, the position of the first recombinase direct repeat corresponds to a position at an L1 site of a reference sequence for the serotype of the packaging sequence, as set forth in any one of Tables 22-27; and the position of the second recombinase direct repeat corresponds to a position that is at an R3 site of a reference sequence for the serotype of the packaging sequence, as set forth in any one of Tables 22-27. In various embodiments, the position of the first recombinase direct repeat corresponds to a position at an L2 site of a reference sequence for the serotype of the packaging sequence, as set forth in any one of Tables 22-27; and the position of the second recombinase direct repeat corresponds to a position that is at an R1 site of a reference sequence for the serotype of the packaging sequence, as set forth in any one of Tables 22-27. In various embodiments, the position of the first recombinase direct repeat corresponds to a position at an L3 site of a reference sequence for the serotype of the packaging sequence, as set forth in any one of Tables 22-27; and the position of the second recombinase direct repeat corresponds to a position that is at an R2 site of a reference sequence for the serotype of the packaging sequence, as set forth in any one of Tables 22-27. In various embodiments, the position of the first recombinase direct repeat corresponds to a position at an L4 site of a reference sequence for the serotype of the packaging sequence, as set forth in any one of Tables 22-27; and the position of the second recombinase direct repeat corresponds to a position that is at an RI site of a reference sequence for the serotype of the packaging sequence, as set forth in any one of Tables 22-27.

In various embodiments, the 5′ ITR and the 3′ ITR are derived from the same serotype. In various embodiments, the 5′ ITR, the 3′ ITR, and the packaging sequence are derived from the same serotype. In various embodiments, the recombinase direct repeats that flank the packaging sequence are FRT, loxP, rox, vox, AttB, or AttP sites. In various embodiments, the recombinase direct repeats that flank the packaging sequence are loxP sites. The present disclosure includes a recombinant adenoviral helper vector including a helper genome of the present disclosure.

In at least one aspect, the present disclosure provides a recombinant adenoviral vector production system including: (i) a helper genome of the present disclosure, and (ii) a helper-dependent adenoviral (HDAd) donor genome, the HDAd donor genome including: a 5′ inverted terminal repeat (ITR); a 3′ ITR; a packaging sequence; and a nucleic acid sequence encoding at least one heterologous expression product; where the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50. In various embodiments, the 5′ ITR and the 3′ ITR of the HDAd donor genome are derived from the same serotype. In various embodiments, the 5′ ITR, the 3′ ITR, and the packaging sequence of the HDAd donor genome are derived from the same serotype.

In at least one aspect, the present disclosure provides a method of producing a recombinant helper-dependent adenoviral (HDAd) donor vector, the method including isolating the recombinant HDAd donor vector from a culture of cells, where the cells include: a recombinant helper genome of the present disclosure or a recombinant adenoviral helper vector of the present disclosure; and a recombinant HDAd donor genome including: a 5′ inverted terminal repeat (ITR); a 3′ ITR; a packaging sequence; and a nucleic acid sequence encoding at least one heterologous expression product; where the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50. In various embodiments, the 5′ ITR and the 3′ ITR of the HDAd donor genome are derived from the same serotype. In various embodiments, the 5′ ITR, the 3′ ITR, and the packaging sequence of the HDAd donor genome are derived from the same serotype.

In various embodiments, a helper genome of the present disclosure includes an inverted packaging sequence.

In at least one aspect, the present disclosure provides a recombinant adenoviral helper genome including: a 5′ inverted terminal repeat (ITR); a 3′ ITR; and a packaging sequence; where the 5′ ITR, the 3′ ITR, and the packaging sequence are each derived from a species B adenovirus of a serotype selected from Ad3, Ad7, Ad11, Ad14, Ad16, Ad21, Ad34, or Ad50; where the packaging sequence is flanked by or includes recombinase direct repeats including a first recombinase direct repeat and a second recombinase direct repeat; where the position of the first recombinase direct repeat corresponds to a position that is within 10 nucleotides of an L1, L2, L3, or L4 site of a reference sequence for the serotype of the packaging sequence, as set forth in Table 21; and where the position of the second recombinase direct repeat corresponds to a position that is within 10 nucleotides of an R1, R2, or R3 site of a reference sequence for the serotype of the packaging sequence, as set forth in Table 21; and where the helper genome includes an inverted packaging sequence. In various embodiments, the 5′ ITR and the 3′ ITR are derived from the same serotype. In various embodiments, the 5′ ITR, the 3′ ITR, and the packaging sequence are derived from the same serotype. In various embodiments, the recombinase direct repeats that flank the packaging sequence are FRT, loxP, rox, vox, AttB, or AttP sites. In various embodiments, the recombinase direct repeats that flank the packaging sequence are loxP sites. The present disclosure includes a recombinant adenoviral helper vector including a helper genome of the present disclosure.

In at least one aspect, the present disclosure provides a recombinant adenoviral vector production system including: (i) a helper genome of the present disclosure or a helper vector of the present disclosure, and (ii) a helper-dependent adenoviral (HDAd) donor genome, the HDAd donor genome including: a 5′ inverted terminal repeat (ITR); a 3′ ITR; a packaging sequence; and a nucleic acid sequence encoding at least one heterologous expression product; where the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50. In various embodiments, the 5′ ITR and the 3′ ITR of the HDAd donor genome are derived from the same serotype. In various embodiments, the 5′ ITR, the 3′ ITR, and the packaging sequence of the HDAd donor genome are derived from the same serotype.

In at least one aspect, the present disclosure provides a method of producing a recombinant helper-dependent adenoviral (HDAd) donor vector, the method including isolating the recombinant HDAd donor vector from a culture of cells, where the cells include: a recombinant helper genome of the present disclosure or a recombinant adenoviral helper vector of the present disclosure; and a recombinant HDAd donor genome including: a 5′ inverted terminal repeat (ITR); a 3′ ITR; an packaging sequence; and a nucleic acid sequence encoding at least one heterologous expression product; where the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50. In various embodiments, the 5′ ITR and the 3′ ITR of the HDAd donor genome are derived from the same serotype. In various embodiments, the 5′ ITR, the 3′ ITR, and the packaging sequence of the HDAd donor genome are derived from the same serotype.

In various embodiments, a helper genome of the present disclosure includes a nucleic acid sequence that encodes an Ad35 fiber knob. In various embodiments, the Ad35 fiber knob includes a mutation that increases affinity with CD46. In various embodiments, the Ad35 fiber knob includes one or more mutations: selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or including each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

In various embodiments, a helper genome of the present disclosure is present in a cell that includes a nucleic acid encoding a recombinase for recombination of the direct repeats. In various embodiments, the recombinase is a Flp, Cre, Dre, Vika, or PhiC31 recombinase. In various embodiments, the cell is a HEK293 cell, optionally where the cell is a HEK293 cell that encodes or expresses Cre recombinase, optionally where the HEK293 cell that encodes or expresses Cre recombinase is a 116 cell.

In various embodiments, an inverted packaging sequence includes a packaging sequence and one or both of a first recombinase direct repeat and a second recombinase direct repeat. In various embodiments, the inverted packaging sequence includes, or includes a first end point at, a nucleotide position corresponding to a position that is within 25 nucleotides of a Left Inversion Point of a reference sequence for the serotype of the packaging sequence (e.g., at the Left Inversion Point, no more than 25 nucleotides 5′ of the Left Inversion Point, and/or no more than 25 nucleotides 3′ of the Left Inversion Point), as set forth in Table 28. In various embodiments, the inverted packaging sequence includes, or includes a first end point at, a nucleotide position corresponding to a position that is within 10 nucleotides of a Left Inversion Point of a reference sequence for the serotype of the packaging sequence, as set forth in Table 28. In various embodiments, the inverted packaging sequence includes, or includes a first end point at, a nucleotide position corresponding to a position at a Left Inversion Point of a reference sequence for the serotype of the packaging sequence, as set forth in Table 28. In various embodiments, the inverted packaging sequence includes, or includes a first end point at, a nucleotide position corresponding to a position at a Left Inversion Point of a reference sequence for the serotype of the packaging sequence, as set forth in any one of Tables 29-35. In various embodiments, the inverted packaging sequence includes, or includes a second end point at, a nucleotide position corresponding to a position that is within 25 nucleotides of a Right Inversion Point of a reference sequence for the serotype of the packaging sequence (e.g., at the Right Inversion Point, no more than 25 nucleotides 5′ of the Right Inversion Point, and/or no more than 25 nucleotides 3′ of the Right Inversion Point), as set forth in Table 28. In various embodiments, the inverted packaging sequence includes, or includes a second end point at, a nucleotide position corresponding to a position that is within 10 nucleotides of a Right Inversion Point of a reference sequence for the serotype of the packaging sequence, as set forth in Table 28. In various embodiments, the inverted packaging sequence includes, or includes a second end point at, a nucleotide position corresponding to a position at a Right Inversion Point of a reference sequence for the serotype of the packaging sequence, as set forth in Table 28. In various embodiments, the inverted packaging sequence includes, or includes a second end point at, a nucleotide position corresponding to a position at a Right Inversion Point of a reference sequence for the serotype of the packaging sequence, as set forth in any one of Tables 29-35.

In at least one aspect, the present disclosure provides a recombinant recombinase site-flanked adenoviral packaging sequence, where recombinase direct repeats flank a packaging sequence, and where the packaging sequence is derived from a species B adenovirus of a serotype selected from Ad3, Ad7, Ad11, Ad14, Ad16, Ad21, Ad34, or Ad50; and where the packaging sequence corresponds to a fragment of an adenoviral genome having: (i) a first end point that corresponds to a position that is within 10 nucleotides of an L1, L2, L3, or L4 site of a reference sequence for the serotype of the packaging sequence, as set forth in Table 21, and (ii) a second end point that corresponds to a position that is within 10 nucleotides of an R1, R2, or R3 site of a reference sequence for the serotype of the packaging sequence, as set forth in Table 21.

In various embodiments, the first end point corresponds to a position that is within 10 nucleotides of an L1 site of a reference sequence for the serotype of the packaging sequence, as set forth in Table 21; and the second end point corresponds to a position that is within 10 nucleotides of an R3 site of a reference sequence for the serotype of the packaging sequence, as set forth in Table 21. In various embodiments, the first end point corresponds to a position that is within 10 nucleotides of an L2 site of a reference sequence for the serotype of the packaging sequence, as set forth in Table 21; and the second end point corresponds to a position that is within 10 nucleotides of an R1 site of a reference sequence for the serotype of the packaging sequence, as set forth in Table 21. In various embodiments, the first end point corresponds to a position that is within 10 nucleotides of an L3 site of a reference sequence for the serotype of the packaging sequence, as set forth in Table 21; and the second end point corresponds to a position that is within 10 nucleotides of an R2 site of a reference sequence for the serotype of the packaging sequence, as set forth in Table 21. In various embodiments, the first end point corresponds to a position that is within 10 nucleotides of an L4 site of a reference sequence for the serotype of the packaging sequence, as set forth in Table 21; and the second end point corresponds to a position that is within 10 nucleotides of an R1 site of a reference sequence for the serotype of the packaging sequence, as set forth in Table 21.

In various embodiments, the first end point corresponds to a position at an L1, L2, L3, or L4 site of a reference sequence for the serotype of the packaging sequence, as set forth in Table 21; and the second end point corresponds to a position at an R1, R2, or R3 site of a reference sequence for the serotype of the packaging sequence, as set forth in Table 21. In various embodiments, the first end point corresponds to a position at an L1 site of a reference sequence for the serotype of the packaging sequence, as set forth in Table 21; and the second end point corresponds to a position at an R3 site of a reference sequence for the serotype of the packaging sequence, as set forth in Table 21. In various embodiments, the first end point corresponds to a position at an L2 site of a reference sequence for the serotype of the packaging sequence, as set forth in Table 21; and the second end point corresponds to a position at an R1 site of a reference sequence for the serotype of the packaging sequence, as set forth in Table 21. In various embodiments, the first end point corresponds to a position at an L3 site of a reference sequence for the serotype of the packaging sequence, as set forth in Table 21; and the second end point corresponds to a position at an R2 site of a reference sequence for the serotype of the packaging sequence, as set forth in Table 21. In various embodiments, the first end point corresponds to a position at an L4 site of a reference sequence for the serotype of the packaging sequence, as set forth in Table 21; and where the second end point corresponds to a position at an R1 site of a reference sequence for the serotype of the packaging sequence, as set forth in Table 21.

In various embodiments, the first end point corresponds to a position at an L1, L2, L3, or L4 site of a reference sequence for the serotype of the packaging sequence, as set forth in any one of Tables 22-27; and the second end point corresponds to a position at an R1, R2, or R3 site of a reference sequence for the serotype of the packaging sequence, as set forth in any one of Tables 22-27. In various embodiments, the first end point corresponds to a position at an L1 site of a reference sequence for the serotype of the packaging sequence, as set forth in any one of Tables 22-27; and the second end point corresponds to a position at an R3 site of a reference sequence for the serotype of the packaging sequence, as set forth in any one of Tables 22-27. In various embodiments, the first end point corresponds to a position at an L2 site of a reference sequence for the serotype of the packaging sequence, as set forth in any one of Tables 22-27; and the second end point corresponds to a position at an R1 site of a reference sequence for the serotype of the packaging sequence, as set forth in any one of Tables 22-27. In various embodiments, the first end point corresponds to a position at an L3 site of a reference sequence for the serotype of the packaging sequence, as set forth in any one of Tables 22-27; and the second end point corresponds to a position at an R2 site of a reference sequence for the serotype of the packaging sequence, as set forth in any one of Tables 22-27. In various embodiments, the first end point corresponds to a position at an L4 site of a reference sequence for the serotype of the packaging sequence, as set forth in any one of Tables 22-27; and the second end point corresponds to a position at an RI site of a reference sequence for the serotype of the packaging sequence, as set forth in any one of Tables 22-27.

In various embodiments, the packaging sequence is present in an adenoviral genome and is inverted, optionally where the packaging sequence is inverted as compared to a 5′ ITR of the adenoviral genome.

In at least one aspect, the present disclosure provides a recombinant adenoviral helper genome including: a 5′ inverted terminal repeat (ITR); a 3′ ITR; and an inverted sequence including a packaging sequence; where the 5′ ITR, the 3′ ITR, and the packaging sequence are each derived from a species B adenovirus of a serotype selected from Ad3, Ad7, Ad11, Ad14, Ad16, Ad21, Ad34, or Ad50; and where the inverted sequence includes, or includes a first end point at, a nucleotide position corresponding to a position within 25 nucleotides of a Left Inversion Point (e.g., within 10 nucleotides of a Left Inversion Point, e.g., at a Left Inversion Point) of a reference sequence for the serotype of the packaging sequence, as set forth in Table 28; and where the inverted sequence includes, or includes a second end point at, a nucleotide position corresponding to a position within 25 nucleotides of a Right Inversion Point (e.g., within 10 nucleotides of a Right Inversion Point, e.g., at a Right Inversion Point) of a reference sequence for the serotype of the packaging sequence, as set forth in Table 28. In various embodiments, the 5′ ITR and the 3′ ITR are derived from the same serotype. In various embodiments, the 5′ ITR, the 3′ ITR, and the packaging sequence are derived from the same serotype. In various embodiments, recombinase direct repeats flank the packaging sequence.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Antibody: As used herein, the term “antibody” refers to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains can be linked to one another by a single disulfide bond, and two other disulfide bonds can connect the heavy chain hinge regions to one another, so that dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is a polyclonal, monoclonal, monospecific, or multispecific antibody (e.g., a bispecific antibody). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-Bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc., as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

Between or From: As used herein, the term “between” refers to content that falls between indicated upper and lower, or first and second, boundaries, inclusive of the boundaries. Thus, for the avoidance of doubt, the term “between” includes values that are exactly the provided upper or lower, or first or second, bound, as well as all values within the provided range. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position and/or identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Accordingly, a provided amino acid or nucleic acid sequence can have, for example, added, removed, inserted, or deleted positions or units that differ from a reference sequence but do not limit the designation of other positions or units as corresponding to the reference. In nucleic acid sequences, for example, exemplary additions or insertions can include restriction enzyme site nucleotides or recombinase site nucleotides. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE. Two sequences can be identified as corresponding if they are identical or if they share substantial identity, e.g., at least 80%, 85%, 90%, 91%, 92%, 93%, 94%, 95%, 96%, 97%, 98%, or 99% identity. In various embodiments, a nucleic acid sequence can correspond to a sequence that is identical or substantially identical (e.g., at least 80%, 85%, 90%, 91%, 92%, 93%, 94%, 95%, 96%, 97%, 98%, or 99% identical) to the complement of the nucleic acid sequence.

Downstream and Upstream: As used herein, the term “downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a composition (e.g., a formulation) necessary to result in a desired physiological change in a subject. Effective amounts are often administered for research purposes.

Engineered: As used herein, the terms “engineered” and “recombinant” are used interchangeably herein to refer to compositions having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment is a sequence having a number of units having a lower bound selected from 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300 monomeric units and an upper bound selected from 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, a host cell is a cell that has been entered by a viral vector, e.g., a vector of the present disclosure or a viral genome thereof, e.g., a viral genome disclosed herein. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, where the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, where the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represent comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof. Without wishing to be bound by any particular embodiment(s), in various embodiments a reference sequence can be a sequence associated with a sequence accession number provided herein, certain of which sequences associated with sequence accession numbers are provided in the below listing of accession sequences.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a subject to which an agent is administered can be interchangeably referred to as a “recipient.” In some instances, a human subject can be interchangeably referred to as a “patient” or “individual.”

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

### BRIEF DESCRIPTION OF THE DRAWINGS

FIG. 1A is a schematic showing alignment of the ‘left end’ sequences of wild type sequences of Ad3 (NCBI accession no. NC_011203) and Ad35 (GenBank accession no. AY128640) (the ‘left end’ being defined by the conventional representation of adenoviral maps, where the major late promoter transcribes the ‘top strand’). Alignment was used to identify putative packaging signals of Ad35 (boxed). Packaging signals A1, A2, A5, and A6 were identified in accordance with terminology set forth in Ostapchuk and Hearing, J Virol. 2001 75:45-51. The table shown in FIG. 1A provides four exemplary positions for placement of a 5′ loxP site, three exemplary positions for placement of a 3′ loxP site, and four exemplary pairings of a position for placement of a 5′ loxP site and a position for placement of a 3′ loxP site. LoxP sites were inserted in the Ad35 genome to the left of the packaging signal A1 at one of four positions indicated by black arrowheads (i.e., after nucleotide numbers 161, 171, 195, or 224) in combination with a loxP sequence inserted to the right of the packaging signal A6, e.g., at positions indicated by open arrowheads (shown after nucleotide numbers 402 or 479). A loxP sequence can also be inserted at a third position (after nucleotide number 497) to the right of the packaging signal A6. The exemplified combinations are further described in Example 1. Because adenoviral sequences of Example 1 were deleted between base pairs 480, 481, or 482 to 3199 to derive E1-deleted replication incompetent vectors, insertion of a loxP sequence after nucleotide 497 can also be described as an insertion at position 3200, which due to the E1 deletion is not as distant from the other insertions as the number would suggest.

FIG. 1B is a schematic showing alignment of the ‘left end’ sequences of wild type sequences of Ad35 (NCBI accession no. AC_000019), Ad3 (NCBI accession no. NC_011203), Ad7 (GenBank accession no. AY601634), Ad11 (NCBI accession no. NC_011202), Ad14 (GenBank accession no. AY803294), Ad16 (GenBank accession no. AY601636), Ad21 (GenBank accession no. AY601633), Ad34 (GenBank accession no. AY737797), and Ad50 (GenBank accession no. AY737798) (the ‘left end’ being defined by the conventional representation of adenoviral maps, where the major late promoter transcribes the ‘top strand’). Highlighted nucleotides are identical to the corresponding aligned nucleotide in Ad35. Alignment was used to identify putative packaging signals of Ad3, Ad7, Ad11, Ad14, Ad16, Ad21, Ad34, and Ad50 (boxed). Packaging signals A1, A2, A5, and A6 were identified in accordance with terminology set forth in Ostapchuk and Hearing, J Virol. 75(1):45-51 (2001). The arrows indicate four exemplary positions for placement of a 5′ recombinase direct repeat (e.g., a loxP site) and two exemplary positions for placement of a 3′ recombinase direct repeat (e.g., a loxP site). Recombinase direct repeats can be inserted in an Ad3, Ad7, Ad11, Ad14, Ad16, Ad21, Ad34, or Ad50 genome at one of four positions indicated by arrows labelled L1, L2, L3 or L4 in combination with a recombinase direct repeat inserted to the right of the packaging signal A6, e.g., at positions indicated by arrows labelled R1 or R2. A loxP sequence can also be inserted at a third position, denoted as R3, to the right of the packaging signal A6. Particular exemplary combinations of recombinase direct repeat positions are further described in Example 1.

FIG. 2A is a schematic showing the ‘left end’ sequence of an Ad35 helper genome which corresponds to that shown in FIG. 1A (see also GenBank accession number AY128640) and includes (i) six nucleotides added to produce an FseI restriction site between Ad35 positions 143 and 144, (ii) loxP sites added after positions 224 and 402, and (iii) I-SceI and FseI sites added after position 480. Certain added sequences are shown within boxes.

FIG. 2B is a schematic showing the ‘left end’ sequence of an Ad35 helper genome which corresponds to that shown in FIG. 1A (see also GenBank accession number AY128640) and includes (i) six nucleotides added to produce an FseI restriction site between Ad35 positions 143 and 144, (ii) loxP sites added after positions 171 and 402, and (iii) I-SceI and FseI sites added after position 480. Certain added sequences are shown within boxes.

FIG. 2C is a schematic showing the ‘left end’ sequence of an Ad35 helper genome which corresponds to that shown in FIG. 1A (see also GenBank accession number AY128640) and includes (i) six nucleotides added to produce an FseI restriction site between Ad35 positions 143 and 144, (ii) loxP sites added after positions 195 and 479, and (iii) I-SceI and FseI sites added after position 480. Certain added sequences are shown within boxes.

FIG. 2D is a schematic showing the ‘left end’ sequence of an Ad35 helper genome which corresponds to that shown in FIG. 1A (see also GenBank accession number AY128640) and includes (i) six nucleotides added to produce a first FseI restriction site between Ad35 positions corresponding to 143 and 144, (ii) loxP sites added after positions corresponding to 161 and 497, (iii) an I-SceI site inserted in place of the canonical sequence at positions corresponding to 481-497, and (iv) a second FseI site added after the position corresponding to 497. Certain added sequences are shown within boxes. The loxP site added after the position corresponding to 497 can alternatively be described as being added at position 3200 in a construct that includes a deletion of nucleotide positions 481 or 482 to 3199, and insertion of the I-SceI and second FseI sites after the position corresponding to 480 (accordingly, this loxP site can further alternatively be described as being added together with the I-SceI and second FseI sites after the position corresponding to 480).

FIG. 2E is a schematic showing the ‘left end’ sequence of an Ad35 helper genome which corresponds to that shown in FIG. 1A (see also GenBank accession number AY128640) and includes sequences added after positions corresponding to 206 and 484 to introduce SwaI restriction sites and loxP sites. Certain added sequences are shown within boxes. pEN024 is a plasmid encoding a helper vector genome that includes the construct of this figure. As noted elsewhere herein, and as applicable throughout, where an inserted sequence (such as a loxP site, to provide one non-limiting example) includes terminal nucleotide positions identical in sequence with reference nucleotides that could be construed as displaced by the insertion, the site of the insertion can be represented, e.g., as occurring after any of such terminal nucleotide positions, or after the last nucleotide that does not correspond to the inserted sequence of interest. Thus, for example, the defining loxP insertion positions of pEN024 could alternative be identified, e.g., as after positions corresponding to 206 and 481.

FIG. 3 is an image of a gel showing digestion of Ad35 helper genomes and plasmids including Ad35 helper genomes, together with a table describing the gel. Lanes 1, 3, 6, and 8 of the gel show BsrGI digestion of helper virus genomes produced using pEN025, pEN026, pEN027, and pEN028, respectively, while lanes 2, 4, 7, and 9 of the gel show digestion of the respective starting plasmids with BsrGI and SwaI. Lane 5 includes a 1 Kb Plus ladder. The accompanying table included in the figure shows that pEN025, pEN026, pEN027, and pEN028 each include a conditional packaging sequence according to the present disclosure, in particular one of the 4 constructs described as Constructs 1˜4 in Example 1 (i.e., pEN025 corresponds to Construct 1 and FIG. 2A, pEN026 corresponds to Construct 2 and FIG. 2B, pEN027 corresponds to Construct 3 and FIG. 2C, and pEN028 corresponds to Construct 4 and FIG. 2D), respectively. Expected band sizes were obtained in all lanes.

FIG. 4 is an image of a gel showing digestion of Ad35 helper genomes, together with a table describing the gel. The accompanying table included in the figure shows the plasmid and cell type used in producing the sample shown in each lane, as well as the expected band size. ApaI digestion produces a 2014 bp fragment from packaging-competent Ad35 genomes (flanked packaging sequence not excised), and a smaller fragment from Ad35 genomes from which a flanked packaging sequence has been excised. Lane 1 includes a 1 Kb Plus ladder. All band sizes were consistent with expectations.

FIG. 5 is a plasmid map depicting the structural organization of plasmid 5427, a plasmid that encodes a helper-dependent genome that includes terminal sequences derived from Ad35. The encoded helper-dependent genome includes a cassette for expression of beta-galactosidase. Digestion of plasmid 5427 with the restriction enzyme PmeI releases the helper-dependent genome from the plasmid backbone. At least because the 5′ and 3′ ends of plasmid 5427 include sequences derived from Ad35, the encoded helper-dependent genome can be packaged into vector particles produced using Ad35 helper genomes of the present disclosure.

FIG. 6A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN025) and a plasmid including a helper-dependent genome that includes terminal sequences derived from Ad35 (plasmid 5427).

FIG. 6B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN026) and a plasmid including a helper-dependent genome that includes terminal sequences derived from Ad35 (plasmid 5427).

FIG. 6C is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN027) and a plasmid including a helper-dependent genome that includes terminal sequences derived from Ad35 (plasmid 5427).

FIG. 6D is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN028) and a plasmid including a helper-dependent genome that includes terminal sequences derived from Ad35 (plasmid 5427).

FIG. 6E is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN024) and a plasmid including a helper-dependent genome that includes terminal sequences derived from Ad35 (plasmid 5427).

FIG. 7A is an image of a gel, together with a table describing the gel. The gel shows digestion of adenoviral genomes obtained from 116 cells transfected with a plasmid including an Ad35 helper genome according to the present disclosure (pEN025 or pEN026) and plasmid 5427, then purified by two successive rounds of cesium chloride gradient purification. As indicated in the table included in the figure, the purified adenoviral genomes were digested with SacII (lanes 4 and 6), while parental plasmids were also digested for comparison (lanes 2, 3, and 5). In lane 2, plasmid 5427 was digested with PmeI (releases the helper-dependent genome from the plasmid 5427 backbone) and SacII. In lanes 3 and 5, helper plasmids pEN025 and pEN026 were digested with SwaI (releases helper genome from the plasmid backbone of pEN025 and pEN026) and SacII.

FIG. 7B is an image of a gel, together with a table describing the gel. The gel shows digestion of adenoviral genomes obtained from 116 cells transfected with a plasmid including an Ad35 helper genome according to the present disclosure (pEN027 or pEN028) and plasmid 5427, then purified by two successive rounds of cesium chloride gradient purification. As indicated in the table included in the figure, the purified adenoviral genomes were digested with SacII (lanes 4 and 6), while parental plasmids were also digested for comparison (lanes 2, 3, and 5). In lane 2, plasmid 5427 was digested with PmeI (releases the helper-dependent genome from the plasmid 5427 backbone) and SacII. In lanes 3 and 5, helper plasmids pEN027 and pEN028 were digested with SwaI (releases helper genome from the plasmid backbone of pEN027 and pEN028) and SacII.

FIG. 7C is an image of a gel, together with a table describing the gel. The gel shows digestion of adenoviral genomes obtained from 116 cells transfected with a plasmid including an Ad35 helper genome according to the present disclosure (pEN024) and plasmid 5427, then purified by two successive rounds of cesium chloride gradient purification. As indicated in the table included in the figure, the purified adenoviral genomes were digested with SacII (lane 4), while parental plasmids were also digested for comparison (lanes 2 and 3). In lane 3, plasmid 5427 was digested with PmeI (releases the helper-dependent genome from the plasmid 5427 backbone) and SacII. In lane 2, helper plasmids pEN024 was digested with PmeI (releases helper genome from the plasmid backbone of pEN024) and SacII.

FIG. 8A is a schematic showing homologous recombination between an Ad35 helper genome (Helper Ad) and a helper-dependent Ad35 genome (HDAd) that results in elimination of one of the recombinase sites that flank a packaging sequence.

FIG. 8B is a schematic showing an Ad35 helper genome (Helper Ad) that includes a packaging sequence inversion. Packaging sequence inversion reduces and/or eliminate recombinase site-excising homologous recombination, and thereby prevents production of a constitutively packageable helper genome.

FIG. 9A is a schematic showing the ‘left end’ sequence of an Ad35 helper genome that includes a packaging sequence inversion. The sequence shown in FIG. 9A corresponds to the sequence of FIG. 2A and includes an inversion of nucleotides positioned between FseI sites of FIG. 2A.

FIG. 9B is a schematic showing the ‘left end’ sequence of an Ad35 helper genome that includes a packaging sequence inversion. The sequence shown in FIG. 9A corresponds to the sequence of FIG. 2B and includes an inversion of nucleotides positioned between FseI sites of FIG. 2B.

FIG. 9C is a schematic showing the ‘left end’ sequence of an Ad35 helper genome that includes a packaging sequence inversion. The sequence shown in FIG. 9C corresponds to the sequence of FIG. 2C and includes an inversion of nucleotides positioned between FseI sites of FIG. 2C.

FIG. 9D is a schematic showing the ‘left end’ sequence of an Ad35 helper genome that includes a packaging sequence inversion. The sequence shown in FIG. 9D corresponds to the sequence of FIG. 2D and includes an inversion of nucleotides positioned between FseI sites of FIG. 2D.

FIG. 10 is an image of a gel showing digestion of Ad35 helper genomes and plasmids including Ad35 helper genomes, together with a table describing the gel. Lanes 2 and 4 of the gel show XmnI digestion of helper virus genomes produced using pEN0056 and pEN0057, respectively, while lanes 1 and 3 of the gel show digestion of the respective starting plasmid with XmnI and SwaI. Lane 5 includes a 1 Kb Plus ladder. The accompanying table included in the figure shows that pEN0056 and pEN0057 each include an inverted conditional packaging sequence according to the present disclosure, in particular one of the constructs described as Constructs 7 and 8 in Example 5, respectively (i.e., pEN0056 corresponds to a plasmid including Construct 7 and pEN0057 corresponds to a plasmid including Construct 8). Expected band sizes were obtained in all lanes.

FIG. 11 is an image of a gel showing digestion of Ad35 helper genomes, together with a table describing the gel. The accompanying table included in the figure shows the plasmid and cell type used in producing the sample shown in each lane, as well as the expected band size. ApaI digestion produces a 2013 bp fragment from packaging-competent Ad35 genomes (inverted flanked packaging sequence not excised), and a smaller fragment from Ad35 genomes from which an inverted flanked packaging sequence has been excised. Lane 1 includes a 1 Kb Plus ladder. All band sizes were consistent with expectations.

FIG. 12 is a plasmid map depicting the structural organization of plasmid 5475, a plasmid that encodes a helper-dependent genome that includes terminal sequences derived from Ad35. The encoded helper-dependent genome includes a cassette for expression of beta-galactosidase. Digestion of plasmid 5475 with the restriction enzyme PmeI releases the helper-dependent genome from the plasmid backbone. At least because the 5′ and 3′ ends of plasmid 5475 include sequences derived from Ad35, the encoded helper-dependent genome can be packaged into vector particles produced using Ad35 helper genomes of the present disclosure.

FIG. 13A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0056) and a plasmid including a helper-dependent genome that includes terminal sequences derived from Ad35 (plasmid 5475).

FIG. 13B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including a helper-dependent genome that includes terminal sequences derived from Ad35 (plasmid 5475).

FIG. 14 is an image of a gel, together with a table describing the gel. The gel shows digestion of adenoviral genomes obtained from 116 cells transfected with a plasmid including an Ad35 helper genome according to the present disclosure (pEN0056 or pEN0057) and plasmid 5475, then purified by two successive rounds of cesium chloride gradient purification. As indicated in the table included in the figure, the purified adenoviral genomes were digested with SacII (lanes 4 and 5), while parental plasmids were also digest for comparison (lanes 2 and 3). In lane 3, plasmid 5475 was digested with PmeI (releases the helper-dependent genome from the plasmid 5475 backbone) and SacII. In lane 2, helper plasmid pEN0057 was digested with PmeI (releases helper genome from the plasmid backbone of pEN0057) and SacII. Digestion of helper plasmid pEN0056 is predicted to display a comparable restriction pattern to that of pEN0057.

FIG. 15A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including an exemplary helper-dependent genome that includes terminal sequences derived from Ad35 (plasmid 1).

FIG. 15B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including an exemplary helper-dependent genome that includes terminal sequences derived from Ad35 (plasmid 2).

FIG. 16 is an image of a gel, together with a table describing the gel. The gel shows digestion of adenoviral genomes obtained from 116 cells transfected with a plasmid including an Ad35 helper genome according to the present disclosure (pEN0057), and plasmid 1 or plasmid 2, then purified by two successive rounds of cesium chloride gradient purification. As indicated in the table included in the figure, the purified adenoviral genomes were digested with EcoRV (lanes 3, 5, and 6), while parental plasmids were also digest for comparison (lanes 2, 4, and 7). In lanes 4 and 7, plasmid 1 or plasmid 2 was digested with PmeI (releases the helper-dependent genome from the plasmid 5475 backbone) and EcoRV. In lane 2, helper plasmid pEN0057 was digested with SwaI (releases helper genome from the plasmid backbone of pEN0057) and EcoRV.

### DETAILED DESCRIPTION

The present disclosure includes adenoviral serotype 3, 7, 11, 14, 16, 21, 34, and 50 (Ad3, Ad7, Ad11, Ad14, Ad16, Ad21, Ad34, and Ad50, respectively) vectors and Ad3, Ad7, Ad11, Ad14, Ad16, Ad21, Ad34, and Ad50 genomes useful in gene therapy. Adenoviruses are large, icosahedral-shaped, non-enveloped viruses. As those of skill in the art are aware, adenoviral serotypes are grouped into adenoviral species known in the art. Seven exemplary adenoviral are species A, B, C, D, E, F, and G. The species C adenoviral serotype 5 (Ad5) is commonly used to generate adenoviral vectors, e.g., for therapeutic use. The present disclosure includes the recognition that there is a need for methods and compositions that support use of alternative adenoviral serotypes, and in particular adenoviral serotypes of other adenoviral species, e.g., for gene therapy. For example, use of certain species B serotypes may be desirable for generating adenoviral vectors and genomes for use in gene therapy. Adenoviral species B serotypes include adenoviral serotypes Ad3, Ad7, Ad11, Ad14, Ad16, Ad21, Ad34, Ad50, and Ad35. The present disclosure further includes the recognition that adenoviral serotypes within a species are more similar (e.g., have more similar genomes and/or packaging sequences, as can be measured for example by sequence identity) than adenoviral serotypes of distinct species, and moreover that for at least this reason methods and compositions disclosed herein can be applied across serotypes of a relevant species (e.g., across species B adenoviral serotypes). While some viral vectors are characterized by relatively high immunogenicity in human populations and/or by relatively low payload capacity, Ad3, Ad7, Ad11, Ad14, Ad16, Ad21, Ad34, and Ad50 vectors are characterized by relatively low immunogenicity in human populations and relatively high payload capacity. However, engineering of Ad3, Ad7, Ad11, Ad14, Ad16, Ad21, Ad34, and Ad50 vectors and genomes for use in gene therapy is not straightforward. The present disclosure includes, among other things, engineering of Ad3, Ad7, Ad11, Ad14, Ad16, Ad21, Ad34, and Ad50 helper vectors useful in producing therapeutic Ad3, Ad7, Ad11, Ad14, Ad16, Ad21, Ad34, and Ad50 donor vectors and/or in methods of gene therapy.

Those of skill in the art will appreciate that, throughout the present disclosure, references to particular nucleotide positions and/or positions corresponding thereto disclose both the specific position identified and similar positions, e.g., positions within 10, 9, 8, 7, 6, 5, 4, 3, 2, or 1 nucleotides of an indicated position. Moreover, in various embodiments in which a heterologous sequence is inserted into or positioned within a sequence corresponding to a reference adenoviral genome, the specific point of insertion can be equivalently referred to by multiple positions if the inserted sequence includes nucleotides adjacent to reference sequence that are the same as would be found in the reference sequence. In various such embodiments, the insertion can be identified as an insertion after any nucleotide position that is contiguous with reference sequence nucleotides and identical in sequence to a corresponding nucleotide of the reference sequence.

Adenoviral genomes such as the Ad3, Ad7, Ad11, Ad14, Ad16, Ad21, Ad34, and Ad50 genomes include DNA flanked on both ends by serotype-specific inverted terminal repeats (ITRs), which are understood to be cis elements that contribute to or are necessary for viral genome replication. ITRs can be, e.g., approximately 100-200 base pairs (e.g., about 160 base pairs) in length, with highest conservation at nucleotide positions (e.g., ˜50 base pairs) closest to the adenoviral genome termini. In various embodiments, an adenoviral genome of the present disclosure includes a 5′ ITR and a 3′ ITR that are derived from the same serotype. In various embodiments, Ad3, Ad7, Ad11, Ad14, Ad16, Ad21, Ad34, and Ad50 ITRs include a 5′ ITR according to SEQ ID NOs: 101, 119, 137, 155, 173, 191, 209, and 227, and a 3′ ITR according to SEQ ID NOs: 102, 120, 138, 156, 174, 192, 210, and 228. In various embodiments, an Ad3, Ad7, Ad11, Ad14, Ad16, Ad21, Ad34, or Ad50 5′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides) having at least 80% sequence identity (e.g., at least 80%, 85%, 90%, 95%, 96%, 97%, 98%, or 99% sequence identity) with a corresponding fragment of nucleotides 1-200 of SEQ ID NOs: 263, 264, 265, 266, 267, 268, 269, or 270, and an Ad3, Ad7, Ad11, Ad14, Ad16, Ad21, Ad34, or Ad50 3′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides) having at least 80% sequence identity (e.g., at least 80%, 85%, 90%, 95%, 96%, 97%, 98%, or 99% sequence identity) with a corresponding fragment of 200 nucleotides at the end of SEQ ID NOs: 263, 264, 265, 266, 267, 268, 269, or 270. In various embodiments, an ITR is sufficient for one or both of Ad35 encapsidation and/or replication. In various embodiments, an Ad7, Ad11, Ad14, Ad16, Ad34, or Ad50 ITR sequence for Ad7, Ad11, Ad14, Ad16, Ad34, or Ad50 vectors differs in that the first 8 bp are CTATCTAT (SEQ ID NO: 12) rather than the canonical sequence at positions 1-8 of a reference Ad7, Ad11, Ad14, Ad16, Ad34, or Ad50 genome sequence (Wunderlich et al., J. Gen Virol. 95:1574-1584 (2014)).

Adenoviral genomes also include a cis-acting packaging sequence (e.g., a conditional or non-conditional packaging sequence, the packaging sequence sometimes represented by the symbol ψ), which can facilitate packaging of the viral genome into viral vectors. In various embodiments, a packaging sequence can be positioned in the 5′ portion of an Ad genome, with the 5′ ITR.

Natural adenoviral genomes encode several proteins including early transcriptional units, E1, E2, E3, and E4 and late transcriptional units which encode structural protein components of the adenoviral vector. Early (E) and late (L) transcription are divided by the onset of viral genome replication. Late transcription includes expression of proteins that make up the viral capsid. Adenoviral capsids include three types of proteins: fiber, penton, and hexon.

In various embodiments, an Ad3, Ad7, Ad11, Ad14, Ad16, Ad21, Ad34, or Ad50 genome is a single-stranded or double-stranded DNA sequence that includes ITRs of an Ad3, Ad7, Ad11, Ad14, Ad16, Ad21, Ad34, or Ad50 vector (e.g., a 5′ ITR according to SEQ ID NOs: 101, 119, 137, 155, 173, 191, 209, or 227, and a 3′ ITR according to SEQ ID NOs: 102, 120, 138, 156, 174, 192, 210, or 228), or ITRs that individually and/or together have at least 75% sequence identity (e.g., at least 75%, 80%, 85%, 90%, 95%, 96%, 97%, 98%, 99%, or 100% identity) thereto.

In various embodiments, an Ad3, Ad7, Ad11, Ad14, Ad16, Ad21, Ad34, or Ad50 genome is a single-stranded or double-stranded DNA sequence that includes a packaging sequence of an Ad3, Ad7, Ad11, Ad14, Ad16, Ad21, Ad34, or Ad50 vector (e.g., a packaging sequence according to SEQ ID NOs: 103, 121, 139, 157, 175, 193, 211, or 229), or a packaging sequence having at least 75% sequence identity (e.g., at least 75%, 80%, 85%, 90%, 95%, 96%, 97%, 98%, 99%, or 100% identity) to the entirety or a portion thereof.

In various embodiments, an Ad3, Ad7, Ad11, Ad14, Ad16, Ad21, Ad34, or Ad50 genome is a single-stranded or double-stranded DNA sequence that includes a sequence with at least 75% sequence identity (e.g., at least 75%, 80%, 85%, 90%, 95%, 96%, 97%, 98%, 99%, or 100% identity) to all, a portion of, or a contiguous corresponding portion of, or a discontiguous corresponding portion of a reference Ad3, Ad7, Ad11, Ad14, Ad16, Ad21, Ad34, or Ad50 genome (e.g., SEQ ID NOs: 263, 264, 265, 266, 267, 268, 269, or 270).

In various embodiments, an Ad3, Ad7, Ad11, Ad14, Ad16, Ad21, Ad34, or Ad50 genome is any nucleotide sequence that includes at least ITRs of an Ad3, Ad7, Ad11, Ad14, Ad16, Ad21, Ad34, or Ad50 vector (e.g., a 5′ ITR according to SEQ ID NOs: 101, 119, 137, 155, 173, 191, 209, or 227, and a 3′ ITR according to SEQ ID NOs: 102, 120, 138, 156, 174, 192, 210, or 228), or ITRs that individually and/or together have at least 75% sequence identity (e.g., at least 75%, 80%, 85%, 90%, 95%, 96%, 97%, 98%, 99%, or 100% identity) thereto.

In various embodiments, an Ad3, Ad7, Ad11, Ad14, Ad16, Ad21, Ad34, or Ad50 genome is an Ad3, Ad7, Ad11, Ad14, Ad16, Ad21, Ad34, or Ad50 genome from which one or more nucleotides, coding sequences, and/or genes are completely or partially deleted as compared to a reference sequence. For example, in some embodiments, an Ad3, Ad7, Ad11, Ad14, Ad16, Ad21, Ad34, or Ad50 genome can be a genome that does not include one or more of an E1, E2, E3, and/or E4 region. In certain embodiments, an Ad3, Ad7, Ad11, Ad14, Ad16, Ad21, Ad34, or Ad50 genome is a genome that does not include any coding sequences of an Ad3, Ad7, Ad11, Ad14, Ad16, Ad21, Ad34, or Ad50 genome (e.g., a “gutless” vector that includes ITRs having at least 75% sequence identity to Ad3, Ad7, Ad11, Ad14, Ad16, Ad21, Ad34, or Ad50 genome ITRs but includes none of the coding sequences present in a reference Ad3, Ad7, Ad11, Ad14, Ad16, Ad21, Ad34, or Ad50 genome).

In various embodiments, an Ad3, Ad7, Ad11, Ad14, Ad16, Ad21, Ad34, or Ad50 genome includes, does not include, or includes a deletion of, all or a portion of an E1 sequence according to SEQ ID NOs: 104, 122, 140, 158, 176, 194, 212, or 230, or a sequence having at least 75% sequence identity (e.g., at least 75%, 80%, 85%, 90%, 95%, 96%, 97%, 98%, 99%, or 100% identity) thereto.

In various embodiments, an Ad3, Ad7, Ad11, Ad14, Ad16, Ad21, Ad34, or Ad50 genome includes, does not include, or includes a deletion of, all or a portion of an E2 sequence according to SEQ ID NOs: 105, 123, 141, 159, 177, 195, 213, or 231, or a sequence having at least 75% sequence identity (e.g., at least 75%, 80%, 85%, 90%, 95%, 96%, 97%, 98%, 99%, or 100% identity) thereto.

In various embodiments, an Ad3, Ad7, Ad11, Ad14, Ad16, Ad21, Ad34, or Ad50 genome includes, does not include, or includes a deletion of, all or a portion of an E3 sequence according to SEQ ID NOs: 106, 124, 142, 160, 178, 196, 214, or 232, or a sequence having at least 75% sequence identity (e.g., at least 75%, 80%, 85%, 90%, 95%, 96%, 97%, 98%, 99%, or 100% identity) thereto.

In various embodiments, an Ad3, Ad7, Ad11, Ad14, Ad16, Ad21, Ad34, or Ad50 genome includes, or does not include, a sequence that encodes a fiber, where the sequence has at least 75% sequence identity (e.g., at least 75%, 80%, 85%, 90%, 95%, 96%, 97%, 98%, 99%, or 100% identity) to SEQ ID NOs: 107, 125, 143, 161, 179, 197, 215, or 233.

In various embodiments, an Ad3, Ad7, Ad11, Ad14, Ad16, Ad21, Ad34, or Ad50 genome includes, or does not include, a sequence that encodes a fiber tail, where the sequence has at least 75% sequence identity (e.g., at least 75%, 80%, 85%, 90%, 95%, 96%, 97%, 98%, 99%, or 100% identity) to SEQ ID NOs: 108, 126, 144, 162, 180, 198, 216, or 234.

In various embodiments, an Ad3, Ad7, Ad11, Ad14, Ad16, Ad21, Ad34, or Ad50 genome includes, or does not include, a sequence that encodes a fiber shaft, where the sequence has at least 75% sequence identity (e.g., at least 75%, 80%, 85%, 90%, 95%, 96%, 97%, 98%, 99%, or 100% identity) to SEQ ID NOs: 109, 127, 145, 163, 181, 199, 217, or 235.

In various embodiments, an Ad3, Ad7, Ad11, Ad14, Ad16, Ad21, Ad34, or Ad50 genome includes, or does not include, a sequence that encodes a fiber knob, where the sequence has at least 75% sequence identity (e.g., at least 75%, 80%, 85%, 90%, 95%, 96%, 97%, 98%, 99%, or 100% identity) to SEQ ID NOs: 110, 128, 146, 164, 182, 200, 218, or 236.

In various embodiments, an Ad3, Ad7, Ad11, Ad14, Ad16, Ad21, Ad34, or Ad50 genome includes, or does not include, a sequence that encodes a penton, where the sequence has at least 75% sequence identity (e.g., at least 75%, 80%, 85%, 90%, 95%, 96%, 97%, 98%, 99%, or 100% identity) to SEQ ID NOs: 111, 129, 147, 165, 183, 201, 219, or 237.

In various embodiments, an Ad3, Ad7, Ad11, Ad14, Ad16, Ad21, Ad34, or Ad50 genome includes, or does not include, a sequence that encodes a hexon, where the sequence has at least 75% sequence identity (e.g., at least 75%, 80%, 85%, 90%, 95%, 96%, 97%, 98%, 99%, or 100% identity) to SEQ ID NOs: 112, 130, 148, 166, 184, 202, 220, or 238.

The present disclosure includes Ad3, Ad7, Ad11, Ad14, Ad16, Ad21, Ad34, or Ad50 vectors that include a fiber having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber (e.g., a fiber according to SEQ ID NOs: 113, 131, 149, 167, 185, 203, 221, or 239).

The present disclosure includes Ad3, Ad7, Ad11, Ad14, Ad16, Ad21, Ad34, or Ad50 vectors that include a fiber shaft having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber shaft (e.g., a fiber shaft according to SEQ ID NOs: 114, 132, 150, 168, 186, 204, 222, or 240).

The present disclosure includes Ad3, Ad7, Ad11, Ad14, Ad16, Ad21, Ad34, or Ad50 vectors that include a fiber knob having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber knob (e.g., a fiber knob according to SEQ ID NOs: 115, 133, 151, 169, 187, 205, 223, or 241).

The present disclosure includes Ad3, Ad7, Ad11, Ad14, Ad16, Ad21, Ad34, or Ad50 vectors that include a penton having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 penton (e.g., a penton according to SEQ ID NOs: 116, 134, 152, 170, 188, 206, 224, or 242).

The present disclosure includes Ad3, Ad7, Ad11, Ad14, Ad16, Ad21, Ad34, or Ad50 vectors that include a hexon having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 hexon (e.g., a hexon according to SEQ ID NOs: 117, 135, 153, 171, 189, 207, 225, or 243).

The present disclosure includes Ad3, Ad7, Ad11, Ad14, Ad16, Ad21, Ad34, or Ad50 vectors that include a fiber tail having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber tail (e.g., a fiber tail according to SEQ ID NOs: 118, 136, 154, 172, 190, 208, 226, or 244, e.g., where the fiber tail is the portion of the fiber including all amino acids N-terminal to the fiber shaft).

In various embodiments, an Ad3, Ad7, Ad11, Ad14, Ad16, Ad21, Ad34, or Ad50 vector is any adenoviral vector that includes at least a fiber having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber (e.g., a fiber according to SEQ ID NOs: 113, 131, 149, 167, 185, 203, 221, or 239), respectively.

In various embodiments, an Ad3, Ad7, Ad11, Ad14, Ad16, Ad21, Ad34, or Ad50 vector is any adenoviral vector that includes at least a fiber shaft having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber shaft (e.g., a fiber shaft according to SEQ ID NOs: 114, 132, 150, 168, 186, 204, 222, or 240), respectively.

In various embodiments, an Ad3, Ad7, Ad11, Ad14, Ad16, Ad21, Ad34, or Ad50 vector is any adenoviral vector that includes at least a fiber knob having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber knob (e.g., a fiber knob according to SEQ ID NOs: 115, 133, 151, 169, 187, 205, 223, or 241), respectively.

In various embodiments, an Ad3, Ad7, Ad11, Ad14, Ad16, Ad21, Ad34, or Ad50 vector is any adenoviral vector that includes at least a penton having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 penton (e.g., a penton according to SEQ ID NOs: 116, 134, 152, 170, 188, 206, 224, or 242), respectively.

In various embodiments, an Ad3, Ad7, Ad11, Ad14, Ad16, Ad21, Ad34, or Ad50 vector is any adenoviral vector that includes at least a hexon having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 hexon (e.g., a hexon according to SEQ ID NOs: 117, 135, 153, 171, 189, 207, 225, or 243), respectively.

In various embodiments, an Ad3, Ad7, Ad11, Ad14, Ad16, Ad21, Ad34, or Ad50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber tail (e.g., a fiber tail according to SEQ ID NOs: 118, 136, 154, 172, 190, 208, 226, or 244, e.g., where the fiber tail is the portion of the fiber including all amino acids N-terminal to the fiber shaft), respectively.

In various embodiments, an Ad3, Ad7, Ad11, Ad14, Ad16, Ad21, Ad34, or Ad50 vector is any adenoviral vector that includes at least 5′ and 3′ ITRs individually and/or together having at least 75% sequence identity (e.g., at least 75%, 80%, 85%, 90%, 95%, 96%, 97%, 98%, 99%, or 100% identity) to ITRs of Ad3, Ad7, Ad11, Ad14, Ad16, Ad21, Ad34, or Ad50 (e.g., a 5′ ITR according to SEQ ID NOs: 101, 119, 137, 155, 173, 191, 209, or 227, and a 3′ ITR according to SEQ ID NOs: 102, 120, 138, 156, 174, 192, 210, or 228), respectively.

Thus, an Ad3, Ad7, Ad11, Ad14, Ad16, Ad21, Ad34, or Ad50 vector can be a chimeric adenoviral vector that includes at least a fiber knob having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber knob and at least one protein or portion thereof (such as a fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, Ad7, Ad11, Ad14, Ad16, Ad21, Ad34, or Ad50 vector can be a chimeric adenoviral vector that includes at least a fiber shaft having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber shaft and at least one protein or portion thereof (such as a fiber knob, fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, Ad7, Ad11, Ad14, Ad16, Ad21, Ad34, or Ad50 vector can be a chimeric adenoviral vector that includes at least a fiber tail having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 fiber tail and at least one protein or portion thereof (such as a fiber knob, fiber shaft, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, Ad7, Ad11, Ad14, Ad16, Ad21, Ad34, or Ad50 vector can be a chimeric adenoviral vector that includes at least a penton having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 penton and at least one protein or portion thereof (such as a fiber knob, fiber shaft, fiber tail, or hexon) that has at least 75% sequence identity (e.g., at least 75%, 80%, 85%, 90%, 95%, 96%, 97%, 98%, 99%, or 100% identity) to a different adenoviral serotype.

An Ad3, Ad7, Ad11, Ad14, Ad16, Ad21, Ad34, or Ad50 vector can be a chimeric adenoviral vector that includes at least a hexon having at least 75% sequence identity (e.g., at least 75%, 80%, 85%, 90%, 95%, 96%, 97%, 98%, 99%, or 100% identity) to an Ad3, Ad7, Ad11, Ad14, Ad16, Ad21, Ad34, or Ad50 hexon and at least one protein or portion thereof (such as a fiber knob, fiber shaft, fiber tail, or penton) that has at least 75% sequence identity (e.g., at least 75%, 80%, 85%, 90%, 95%, 96%, 97%, 98%, 99%, or 100% identity) to a different adenoviral serotype.

Ad35 fiber is a fiber protein trimer, each fiber protein includes an N-terminal tail domain that interacts with the pentameric penton base, a C-terminal globular knob domain (fiber knob) that functions as the attachment site for the host cell receptors, and a central shaft domain that connects the tail and the knob domains (shaft). In various embodiments, an Ad35 fiber knob has at least 80% sequence identity (e.g., at least 80%, 85%, 90%, 95%, 96%, 97%, 98%, or 99% sequence identity) with reference fiber sequence NCBI accession no. AP_000601. In various embodiments, an Ad35 fiber knob includes amino acids 123 to 320 or 323 of a canonical wild-type Ad35 fiber protein. In various embodiments, an Ad35 fiber knob includes at least 60 amino acids (e.g., at least 60, 70, 80, 90, 100, 110, 120, 130, 140, 150, 160, 170, 180, 190, or 198 amino acids) having at least 80% sequence identity (e.g., at least 80%, 85%, 90%, 95%, 96%, 97%, 98%, or 99% sequence identity) with a corresponding fragment of amino acids 123 to 320 or 323 of a canonical wild-type Ad35 fiber protein.

In various embodiments, a vector such as a helper vector or donor vector includes, or a helper genome encodes, fiber knob mutations as compared to a reference or canonical Ad35 fiber knob, where the mutations increase affinity of the vector, fiber, and/or fiber knob with CD46 (see, e.g. Table 1). In various embodiments, an adenoviral vector such as a helper vector or donor vector includes, or an Ad35 helper genome encodes, an Ad35++ mutant fiber knob. An Ad35++ mutant fiber knob is a fiber knob that includes mutations as compared to a reference or canonical Ad35 fiber knob, where the mutations increase affinity with CD46, e.g., optionally wherein the increase is an increase of up to or at least 1.1-fold, e.g., up to at least 1, 2, 3, 4, 5, 10, 15, 20, or 25-fold. Increased affinity with CD46 can increase efficiency of target cell transduction and/or decrease the multiplicity of infection (MOI) required to achieve a target level of transduction (Li and Lieber, FEBS Letters, 593(24):3623-3648 (2019)). In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to NCBI accession no. AP_000601 or an amino acid sequence corresponding thereto, e.g., where position 207 is Glu or Asp. In various embodiments, an Ad35 fiber has an amino acid sequence according to NCBI accession no. AP_000601. Further description of Ad35++ fiber knob mutations is found in Wang et al., J. Virol. 82(21):10567-10579 (2008), which is incorporated herein by reference in its entirety and with respect to fiber knobs. The present disclosure includes, for example, a recombinant Ad3, Ad7, Ad11, Ad14, Ad16, Ad21, Ad34, or Ad50 vector with an Ad35++ mutant fiber knob or chimeric Ad3, Ad7, Ad11, Ad14, Ad16, Ad21, Ad34, or Ad50 vector with an Ad35++ mutant fiber knob.

TABLE 1
Mutated Ad35 Knob increased binding to CD46

| | Kd | |
|---|---|---|
| A1: Asn217Asp Thr245Pro Ile256Leu* | A1 4.82 nM | |
| A2: Asp207Gly Thr245Ala* | A2 0.629 nM | |
| A3: Asp207Gly Thr226Ala* | A3 1.407 nM | |
| A8: Ile192Val Ile256Val | A8 13.6850 nM | |
| B1: Asp207Gly* | B1 1.774 nM | |
| B2: wtAd35(207Asp) | B2 14.98 nM | |
| B3: Asn217Asp* | B3 16.85 nM | |
| B4: Thr245Ala* | B4 7.64 nM | |
| B5: Ile256Leu* | B5 10.96 nM | |
| B6: Ad3 | B6 no binding | |
| B7: Ad11 | B7 11.22 nM | |
| M1: Arg279Cys** | M1 no binding | |
| M3: Arg279His** | M3 no binding | |
| wtAd35* | 13.7 | nM |
| wtAd35* | 15.36 | nM |
| AA: Asp207Gly Thr245Ala Ile256Leu* | 0.943 | nM |

*Published in Wang et al., J. Virol. 82(21): 10567-10579 (2008)
**Published in Wang et al., J. Virol. 81(23): 12785-12792 (2007)

Helper genomes encode various proteins required for viral particle production. In various embodiments, a helper genome can include one or more, or all, adenoviral genes and/or encode one or more, or all, adenoviral proteins encoded and/or expressed by a reference genome of the helper genome serotype, excluding, e.g., those removed from the helper genome by an E1 region deletion, E2 region deletion, E3 region deletion, and/or E4 region deletion, and/or by any other deletions.

Exemplary sequences of Ad3, Ad7, Ad11, Ad14, Ad16, Ad21, Ad34, and Ad50 components (e.g., ITRs, packaging sequences, genes, and proteins) are provided in the following tables. Viral polypeptides include proteins that are components of viral vectors and portions or fragments thereof, and examples of viral polypeptides therefore include, for example a fiber, fiber knob, fiber shaft, fiber tail, penton, or hexon. An exemplary listing of adenoviral genes and proteins is provided in Table 20 for Ad35. Table 20 relates to an exemplary genome of a natural Ad35 adenovirus (see, e.g., Gao et al., 2003 Gene Ther. 10(23): 1941-9; Reddy et al. 2003 Virology 311(2): 384-393; GenBank accession no. AY128640). Other examples of Ad35 reference genomes can include NCBI accession no. AC_000019 and GenBank accession nos. AY271307 and AX049983.

Various sequences corresponding to accession numbers disclosed herein, including e.g., sequences associated with accession numbers referred to herein as SEQ ID NOs: 263, 264, 265, 266, 267, 268, 269, 270, and/or 271 as indicated in Tables 2-19 are provided herein in the below listing of accession sequences. Those of skill in the art will appreciate that such sequences, including the sequences disclosed in the below listing of accession sequences, can be referenced in whole (e.g., by an accession number) or in part (e.g., by reference to a nucleotide position and/or a set or range of nucleotide positions of a sequence and/or accession number). Additionally, those of skill in the art will appreciate that a reference to a particular serotype can be interpreted as a reference to a corresponding reference sequence (e.g., an accession sequence) of the serotype; and similarly, a reference to a reference sequence (e.g., an accession sequence) of a particular serotype can be interpreted as a reference to the corresponding serotype.

TABLE 2
Ad3 Genomic Sequences
Ad3 Genomic Sequences
Reference Ad3 Genome Sequence: NCBI accession
no. NC_011203 (SEQ ID NO: 263)

| | Exemplary Sequence | SEQ |
|---|---|---|
| Component | (position in reference) | ID NO: |
| Ad3 5′ (left) ITR | 1-136 | 101 |
| Ad3 3′ (right) ITR | 35208-35343 | 102 |
| Ad3 Packaging | 137-479 | 103 |
| Sequence | | |
| Ad3 E1 | 480-3918 | 104 |
| Ad3 E2 | 26643-3947 | 105 |
| Ad3 E3 | 27085-31186 | 106 |
| Ad3 fiber | 31368-32327 | 107 |
| Ad3 fiber tail | 31368-31493 | 108 |
| Ad3 fiber shaft | 31494-31763 | 109 |
| Ad3 fiber knob | 31764-32324 | 110 |
| Ad3 penton | 13905-15539 | 111 |
| Ad3 hexon | 18418-21252 | 112 |

TABLE 3
Ad3 Amino Acid Sequences
Ad3 Amino Acid Sequences

| | Exemplary Sequence | SEQ |
|---|---|---|
| Component | (position in reference) | ID NO: |
| Ad3 fiber | 1-319 (NCBI accession no. YP_002213796) | 113 |
| Ad3 fiber shaft | 43-132 (NCBI accession no. YP_002213796) | 114 |
| Ad3 fiber knob | 134-319 (NCBI accession no. YP_002213796) | 115 |
| Ad3 penton | 1-544 (NCBI accession no. YP_002213774) | 116 |
| Ad3 hexon | 1-944 (NCBI accession no. YP_002213779) | 117 |
| Ad3 fiber tail | 1-42 (NCBI accession no. YP_002213796) | 118 |

TABLE 4
Ad7 Genomic Sequences
Ad7 Genomic Sequences
Reference Ad7 Genome Sequence: GenBank
accession no. AY601634 (SEQ ID NO: 264)

| | Exemplary Sequence | SEQ |
|---|---|---|
| Component | (position in reference) | ID NO: |
| Ad7 5′ (left) ITR | 1-136 | 119 |
| Ad7 3′ (right) ITR | 35063-35198 | 120 |
| Ad7 Packaging Sequence | 137-479 | 121 |
| Ad7 E1 | 480-3918 | 122 |
| Ad7 E2 | 26554-3946 | 123 |
| Ad7 E3 | 26995-31030 | 124 |
| Ad7 fiber | 31214-32191 | 125 |
| Ad7 fiber tail | 31214-31339 | 126 |
| Ad7 fiber shaft | 31340-31612 | 127 |
| Ad7 fiber knob | 31613-32188 | 128 |
| Ad7 penton | 13855-15489 | 129 |
| Ad7 hexon | 18353-21157 | 130 |

TABLE 5
Ad7 Amino Acid Sequences
Ad7 Amino Acid Sequences

| | Exemplary Sequence | SEQ |
|---|---|---|
| Component | (position in reference) | ID NO: |
| Ad7 fiber | 1-325 (GenBank accession no. AP_000564) | 131 |
| Ad7 fiber shaft | 43-133 (GenBank accession no. AP_000564) | 132 |
| Ad7 fiber knob | 134-325 (GenBank accession no. AP_000564) | 133 |
| Ad7 penton | 1-544 (GenBank accession no. AP_000543) | 134 |
| Ad7 hexon | 1-934 (GenBank accession no. AP_000548) | 135 |
| Ad7 fiber tail | 1-42 (GenBank accession no. AP_000564) | 136 |

TABLE 6
Ad11 Genomic Sequences
Ad11 Genomic Sequences
Reference Ad11 Genome Sequence: NCBI
accession no. NC_011202 (SEQ ID NO: 265)

| | Exemplary Sequence | SEQ |
|---|---|---|
| Component | (position in reference) | ID NO: |
| Ad11 5′ (left) ITR | 1-137 | 137 |
| Ad11 3′ (right) ITR | 34658-34794 | 138 |
| Ad11 Packaging Sequence | 138-479 | 139 |
| Ad11 E1 | 480-3931 | 140 |
| Ad11 E2 | 25445-3963 | 141 |
| Ad11 E3 | 26866-30624 | 142 |
| Ad11 fiber | 30811-31788 | 143 |
| Ad11 fiber tail | 30811-30936 | 144 |
| Ad11 fiber shaft | 30937-31209 | 145 |
| Ad11 fiber knob | 31210-31785 | 146 |
| Ad11 penton | 13682-15367 | 147 |
| Ad11 hexon | 18254-21100 | 148 |

TABLE 7
Ad11 Amino Acid Sequences
Ad11 Amino Acid Sequences

| | Exemplary Sequence | SEQ |
|---|---|---|
| Component | (position in reference) | ID NO: |
| Ad11 fiber | 1-325 (GenBank accession no. YP_002213828) | 149 |
| Ad11 fiber | 43-133 (GenBank accession no. YP_002213828) | 150 |
| shaft | | |
| Ad11 fiber | 134-325 (GenBank accession no. | 151 |
| knob | YP_002213828) | |
| Ad11 penton | 1-561 (GenBank accession no. YP_002213807) | 152 |
| Ad11 hexon | 1-948 (GenBank accession no. YP_002213812) | 153 |
| Ad11 fiber | 1-42 (GenBank accession no. YP_002213828) | 154 |
| tail | | |

TABLE 8
Ad14 Genomic Sequences
Ad14 Genomic Sequences
Reference Ad14 Genome Sequence: GenBank
accession no. AY803294 (SEQ ID NO: 266)

| | Exemplary Sequence | SEQ |
|---|---|---|
| Component | (position in reference) | ID NO: |
| Ad14 5′ (left) ITR | 1-137 | 155 |
| Ad14 3′ (right) ITR | 34628-34764 | 156 |
| Ad14 Packaging Sequence | 138-479 | 157 |
| Ad14 E1 | 480-3947 | 158 |
| Ad14 E2 | 23389-3963 | 159 |
| Ad14 E3 | 26854-30601 | 160 |
| Ad14 fiber | 30788-31765 | 161 |
| Ad14 fiber tail | 30788-30913 | 162 |
| Ad14 fiber shaft | 30914-31186 | 163 |
| Ad14 fiber knob | 31187-31762 | 164 |
| Ad14 penton | 13698-15374 | 165 |
| Ad14 hexon | 18252-21089 | 166 |

TABLE 9
Ad14 Amino Acid Sequences
Ad14 Amino Acid Sequences

| | Exemplary Sequence | SEQ |
|---|---|---|
| Component | (position in reference) | ID NO: |
| Ad14 fiber | 1-325 (GenBank accession no. AAW33140) | 167 |
| Ad14 fiber | 43-133 (GenBank accession no. AAW33140) | 168 |
| shaft | | |
| Ad14 fiber | 134-325 (GenBank accession no. AAW33140) | 169 |
| knob | | |
| Ad14 penton | 1-558 (GenBank accession no. AAW33119) | 170 |
| Ad14 hexon | 1-945 (GenBank accession no. AAW33124) | 171 |
| Ad14 fiber | 1-42 (GenBank accession no. AAW33140) | 172 |
| tail | | |

TABLE 10
Ad16 Genomic Sequences
Ad16 Genomic Sequences
Reference Ad16 Genome Sequence: GenBank
accession no. AY601636 (SEQ ID NO: 267)

| | Exemplary Sequence | SEQ |
|---|---|---|
| Component | (position in reference) | ID NO: |
| Ad16 5′ (left) ITR | 1-114 | 173 |
| Ad16 3′ (right) ITR | 35409-35522 | 174 |
| Ad16 Packaging Sequence | 115-479 | 175 |
| Ad16 E1 | 480-3910 | 176 |
| Ad16 E2 | 23580-3954 | 177 |
| Ad16 E3 | 27107-31263 | 178 |
| Ad16 fiber | 31448-32509 | 179 |
| Ad16 fiber tail | 31448-31573 | 180 |
| Ad16 fiber shaft | 31574-31933 | 181 |
| Ad16 fiber knob | 31934-32506 | 182 |
| Ad16 penton | 13902-17534 | 183 |
| Ad16 hexon | 18450-21272 | 184 |

TABLE 11
Ad16 Amino Acid Sequences
Ad16 Amino Acid Sequences

| | Exemplary Sequence | SEQ |
|---|---|---|
| Component | (position in reference) | ID NO: |
| Ad16 fiber | 1-353 (GenBank accession no. AAW33461) | 185 |
| Ad16 fiber | 43-172 (GenBank accession no. AAW33461) | 186 |
| shaft | | |
| Ad16 fiber | 173-353 (GenBank accession no. AAW33461) | 187 |
| knob | | |
| Ad16 penton | 1-555 (GenBank accession no. AAW33439) | 188 |
| Ad16 hexon | 1-940 (GenBank accession no. AAW33444) | 189 |
| Ad16 fiber | 1-42 (GenBank accession no. AAW33461) | 190 |
| tail | | |

TABLE 12
Ad21 Genomic Sequences
Ad21 Genomic Sequences
Reference Ad21 Genome Sequence: GenBank
accession no. AY601633 (SEQ ID NO: 268)

| | Exemplary Sequence | SEQ |
|---|---|---|
| Component | (position in reference) | ID NO: |
| Ad21 5′ (left) ITR | 1-114 | 191 |
| Ad21 3′ (right) ITR | 35269-35382 | 192 |
| Ad21 Packaging Sequence | 115-479 | 193 |
| Ad21 E1 | 480-3911 | 194 |
| Ad21 E2 | 23611-3924 | 195 |
| Ad21 E3 | 27441-31208 | 196 |
| Ad21 fiber | 31406-32377 | 197 |
| Ad21 fiber tail | 31406-31531 | 198 |
| Ad21 fiber shaft | 31532-31804 | 199 |
| Ad21 fiber knob | 31805-32374 | 200 |
| Ad21 penton | 13878-15563 | 201 |
| Ad21 hexon | 18454-21303 | 202 |

TABLE 13
Ad21 Amino Acid Sequences
Ad21 Amino Acid Sequences

| | Exemplary Sequence | SEQ |
|---|---|---|
| Component | (position in reference) | ID NO: |
| Ad21 fiber | 1-323 (GenBank accession no. AAW33370) | 203 |
| Ad21 fiber | 43-133 (GenBank accession no. AAW33370) | 204 |
| shaft | | |
| Ad21 fiber | 134-323 (GenBank accession no. AAW33370) | 205 |
| knob | | |
| Ad21 penton | 1-561 (GenBank accession no. AAW33349) | 206 |
| Ad21 hexon | 1-949 (GenBank accession no. AAW33354) | 207 |
| Ad21 fiber | 1-42 (GenBank accession no. AAW33370) | 208 |
| tail | | |

TABLE 14
Ad34 Genomic Sequences
Ad34 Genomic Sequences
Reference Ad34 Genome Sequence: GenBank
accession no. AY737797 (SEQ ID NO: 269)

| | Exemplary Sequence | SEQ |
|---|---|---|
| Component | (position in reference) | ID NO: |
| Ad34 5′ (left) ITR | 1-137 | 209 |
| Ad34 3′ (right) ITR | 34639-34775 | 210 |
| Ad34 Packaging Sequence | 138-479 | 211 |
| Ad34 E1 | 480-3929 | 212 |
| Ad34 E2 | 23399-3945 | 213 |
| Ad34 E3 | 27185-30625 | 214 |
| Ad34 fiber | 30812-31783 | 215 |
| Ad34 fiber tail | 30812-30937 | 216 |
| Ad34 fiber shaft | 30938-31210 | 217 |
| Ad34 fiber knob | 31211-31780 | 218 |
| Ad34 penton | 13681-15357 | 219 |
| Ad34 hexon | 18244-21099 | 220 |

TABLE 15
Ad34 Amino Acid Sequences
Ad34 Amino Acid Sequences

| | Exemplary Sequence | SEQ |
|---|---|---|
| Component | (position in reference) | ID NO: |
| Ad34 fiber | 1-323 (GenBank accession no. AAW33501) | 221 |
| Ad34 fiber | 43-133 (GenBank accession no. AAW33501) | 222 |
| shaft | | |
| Ad34 fiber | 134-323 (GenBank accession no. AAW33501) | 223 |
| knob | | |
| Ad34 penton | 1-558 (GenBank accession no. ABC49791) | 224 |
| Ad34 hexon | 1-951 (GenBank accession no. AAW33485) | 225 |
| Ad34 fiber | 1-42 (GenBank accession no. AAW33501) | 226 |
| tail | | |

TABLE 16
Ad50 Genomic Sequences
Ad50 Genomic Sequences
Reference Ad50 Genome Sequence: GenBank
accession no. AY737798 (SEQ ID NO: 270)

| | Exemplary Sequence | SEQ |
|---|---|---|
| Component | (position in reference) | ID NO: |
| Ad50 5′ (left) ITR | 1-114 | 227 |
| Ad50 3′ (right) ITR | 35272-35385 | 228 |
| Ad50 Packaging Sequence | 115-479 | 229 |
| Ad50 E1 | 480-3910 | 230 |
| Ad50 E2 | 23590-3923 | 231 |
| Ad50 E3 | 27102-31222 | 232 |
| Ad50 fiber | 31409-32380 | 233 |
| Ad50 fiber tail | 31409-31534 | 234 |
| Ad50 fiber shaft | 31535-31807 | 235 |
| Ad50 fiber knob | 31808-32377 | 236 |
| Ad50 penton | 13888-15570 | 237 |
| Ad50 hexon | 18460-21282 | 238 |

TABLE 17
Ad50 Amino Acid Sequences
Ad50 Amino Acid Sequences

| | Exemplary Sequence | SEQ |
|---|---|---|
| Component | (position in reference) | ID NO: |
| Ad50 fiber | 1-323 (GenBank accession no. AAW33547) | 239 |
| Ad50 fiber | 43-133 (GenBank accession no. AAW33547) | 240 |
| shaft | | |
| Ad50 fiber | 134-323 (GenBank accession no. AAW33547) | 241 |
| knob | | |
| Ad50 penton | 1-560 (GenBank accession no. AAW33525) | 242 |
| Ad50 hexon | 1-940 (GenBank accession no. AAW33530) | 243 |
| Ad50 fiber | 1-42 (GenBank accession no. AAW33547) | 244 |
| tail | | |

TABLE 18
Ad35 Genomic Sequences
Ad35 Genomic Sequences
Reference Ad35 Genome Sequence: GenBank
accession no. AY128640 (SEQ ID NO: 271)

| | Exemplary Sequence | SEQ |
|---|---|---|
| Component | (position in reference) | ID NO: |
| Ad35 5′ (left) ITR | 1-137 | 245 |
| Ad35 3′ (right) ITR | 34658-34794 | 246 |
| Ad35 Packaging Sequence | 138-479 | 247 |
| Ad35 E1 | 480-3400 | 248 |
| Ad35 E2 | 3966-23415 | 249 |
| Ad35 E3 | 27198-30622 | 250 |
| Ad35 fiber | 30826-31797 | 251 |
| Ad35 fiber tail | 30826-30951 | 252 |
| Ad35 fiber shaft | 30952-31224 | 253 |
| Ad35 fiber knob | 31225-31797 | 254 |
| Ad35 penton | 13690-15375 | 255 |
| Ad35 hexon | 18255-21113 | 256 |

TABLE 19
Ad35 Amino Acid Sequences
Ad35 Amino Acid Sequences

| | Exemplary Sequence | SEQ |
|---|---|---|
| Component | (position in reference) | ID NO: |
| Ad35 fiber | 1-323 (NCBI accession no. AP_000601) | 257 |
| Ad35 fiber | 43-133 (NCBI accession no. AP_000601) | 258 |
| shaft | | |
| Ad35 fiber | 134-323 (NCBI accession no. AP_000601) | 259 |
| knob | | |
| Ad35 penton | 1-561 (NCBI accession no. AP_000580) | 260 |
| Ad35 hexon | 1-952 (NCBI accession no. AP_000585) | 261 |
| Ad35 fiber | 1-42 (NCBI accession no. AP_000601) | 262 |
| tail | | |

TABLE 20
Predicted translational features of the Ad35 genome

| Features | From | To |
|---|---|---|
| 5′ (left) ITR | 1 | 137 |
| Packaging Sequence | 138 | 479 |

E1 and pIX regions (E1: 480-3400)

| | | | |
|---|---|---|---|
| E1A 261R | | 569 | 1148 |
| | Join* | 1233 | 1441 |
| E1A 230R | | 569 | 1055 |
| | Join | 1233 | 1441 |
| E1A 58R | | 569 | 640 |
| | Join | 1233 | 1337 |
| EIB 214R (small T antigen) | | 1611 | 2153 |
| EIB 494R (large T antigen) | | 1916 | 3400 |
| pIX | | 3484 | 3903 |
| ORF-1 | | 2366 | 2689 |

E2 and IVa2 regions (complementary strand) (E2: 3966-23415)

| | | | |
|---|---|---|---|
| IVa2 | | 5579 | 5590 |
| | Join | 3966 | 5300 |
| E2B DNA pol | | 5069 | 8437 |
| E2B pTP | | 8440 | 10 356 |
| E2A DBP | | 22 414 | 23 415 |
| ORF-2 | | 5988 | 6482 |
| ORF-3 | | 7847 | 8257 |
| ORF-4 | | 15 663 | 15 971 |
| ORF-5 | | 15 743 | 16 216 |
| ORF-6 | | 16 457 | 17 041 |
| ORF-7 | | 17 543 | 17 938 |
| ORF-8 | | 17 994 | 18 713 |
| ORF-9 | | 21 858 | 22 436 |
| ORF-10 | | 22 128 | 22 502 |
| ORF-11 | | 23 027 | 23 488 |

E3 region (E3: 27198-30622)

| | | |
|---|---|---|
| E3 12.2K protein | 27 198 | 27 515 |
| E3 15.0K protein | 27 469 | 27 864 |
| E3 18.5K protein | 27 849 | 28 349 |
| E3 20.3K protein | 28 369 | 28 914 |
| E3 20.6K protein | 28 932 | 29 495 |
| E3 15.2K protein | 29 817 | 30 221 |
| E3 15.3K protein | 30 214 | 30 621 |
| ORF-12 | 25 693 | 26 019 |
| ORF-13 | 27 908 | 28 240 |

E4 region (complementary strand)

| | | |
|---|---|---|
| E4 299R | 32 075 | 32 974 |
| E4 145R | 33 604 | 34 041 |
| E4 125R | 34 038 | 34 415 |
| E4 117R | 33 254 | 33 607 |
| E4 122R | 32 877 | 33 245 |
| ORF-14 | 33 100 | 33 609 |
| VARNA region | 10 433 | 10 594 |

L region

| | | | |
|---|---|---|---|
| L1 52, 5SK | | 10 653 | 11 819 |
| L1 IIIa | | 11 845 | 13 608 |
| L2 III (penton) | | 13 690 | 15 375 |
| L2 pVII | | 15 383 | 15 961 |
| L2 V | | 16 004 | 17 059 |
| L3 pVI | | 17 399 | 18 139 |
| L3 II (hexon) | | 18 255 | 21 113 |
| L3 23K (protease) | | 21 150 | 21 779 |
| L4 100K | | 23 446 | 25 884 |
| L4 22K | | 25 616 | 26 191 |
| L4 33K | | 25 616 | 25 934 |
| | Join | 26 104 | 26 465 |
| L4 pVIII | | 26 515 | 27 198 |
| L5 IV(fiber) | | 30 826 | 31 797 |
| Fiber Tail | | 30826 | 30951 |
| Fiber Knob | | 30952 | 31224 |
| Fiber Shaft | | 31225 | 31797 |
| 3′ (right) ITR | | 34658 | 34794 |

*“Join” indicates that corresponding mRNA sequences are joined by splicing

Another example of an Ad7 reference genome can include NCBI accession no. AC_000018 (SEQ ID NO: 272). In some embodiments, an Ad7 5′ (left) ITR corresponds to positions 1-136 in NCBI accession no. AC_000018 (SEQ ID NO: 273). In some embodiments, an Ad7 3′ (right) ITR corresponds to positions 35379-35514 in NCBI accession no. AC_000018 (SEQ ID NO: 274). In some embodiments, an Ad7 Packaging Sequence corresponds to positions 137-479 in NCBI accession no. AC_000018 (SEQ ID NO: 275). In some embodiments, an Ad7 E1 corresponds to positions 480-3919 in NCBI accession no. AC 000018 (SEQ ID NO: 276). In some embodiments, an Ad7 E2 corresponds to positions 26867-3947 in NCBI accession no. AC_000018 (SEQ ID NO: 277). In some embodiments, an Ad7 E3 corresponds to positions 27308-31345 in NCBI accession no. AC_000018 (SEQ ID NO: 278). In some embodiments, an Ad7 fiber corresponds to positions 31529-32506 in NCBI accession no. AC_000018 (SEQ ID NO: 279). In some embodiments, an Ad7 fiber tail corresponds to positions 31529-31654 in NCBI accession no. AC_000018 (SEQ ID NO: 280). In some embodiments, an Ad7 fiber shaft corresponds to positions 31655-31927 in NCBI accession no. AC_000018 (SEQ ID NO: 281). In some embodiments, an Ad7 fiber knob corresponds to positions 31928-32503 in NCBI accession no. AC_000018 (SEQ ID NO: 282). In some embodiments, an Ad7 penton corresponds to positions 14153-15787 in NCBI accession no. AC_000018 (SEQ ID NO: 283). In some embodiments, an Ad7 hexon corresponds to positions 18666-21470 in NCBI accession no. AC_000018 (SEQ ID NO: 284).

Another example of an Ad35 reference genome can include NCBI accession no. AC_000019 (SEQ ID NO: 285). In some embodiments, an Ad35 5′ (left) ITR corresponds to positions 1-137 in NCBI accession no. AC_000019 (SEQ ID NO: 286). In some embodiments, an Ad35 3′ (right) ITR corresponds to positions 34658-34794 in NCBI accession no. AC_000019 (SEQ ID NO: 287). In some embodiments, an Ad35 Packaging Sequence corresponds to positions 138-479 in NCBI accession no. AC_000019 (SEQ ID NO: 288). In some embodiments, an Ad35 E1 corresponds to positions 480-3400 in NCBI accession no. AC_000019 (SEQ ID NO: 289). In some embodiments, an Ad35 E2 corresponds to positions 3966-23416 in NCBI accession no. AC_000019 (SEQ ID NO: 290). In some embodiments, an Ad35 E3 corresponds to positions 27199-30622 in NCBI accession no. AC_000019 (SEQ ID NO: 291). In some embodiments, an Ad35 fiber corresponds to positions 30827-31798 in NCBI accession no. AC_000019 (SEQ ID NO: 292). In some embodiments, an Ad35 fiber tail corresponds to positions 30827-30952 in NCBI accession no. AC_000019 (SEQ ID NO: 293). In some embodiments, an Ad35 fiber shaft corresponds to positions 30953-31225 in NCBI accession no. AC_000019 (SEQ ID NO: 294). In some embodiments, an Ad35 fiber knob corresponds to positions 31226-31798 in NCBI accession no. AC_000019 (SEQ ID NO: 295). In some embodiments, an Ad35 penton corresponds to positions 13690-15375 in NCBI accession no. AC_000019 (SEQ ID NO: 296). In some embodiments, an Ad35 hexon corresponds to positions 18257-21115 in NCBI accession no. AC_000019 (SEQ ID NO: 297).

Ad3, Ad7, Ad11, Ad14, Ad16, Ad21, Ad34, and Ad50 genomes and vectors can be engineered for therapeutic use. One goal of certain such engineering can include rendering viral genomes and vectors deficient for propagation in a recipient cell or system, such as a human subject. Propagation deficiency increases the safety of administering the viral genome or vector to the recipient cell or system. Broadly, there are three recognized “generations” of adenoviral vectors and genomes engineered to reduce and/or eliminate replication of the virus in recipients. First-generation adenoviral vectors are engineered to remove genes E1 and E3. Without these genes, adenoviral vectors cannot replicate on their own but can be produced in E1-expressing mammalian cell lines (e.g., HEK293 cells). With only first-generation modifications, adenoviral vector cloning capacity is limited, and host immune response against the vector can be problematic for effective payload expression. Second-generation adenoviral vectors, in addition to E1/E3 removal, are engineered to remove non-structural genes E2 and E4, resulting in increased capacity and reduced immunogenicity. Third-generation adenoviral vectors (also referred to as gutless, high capacity adenoviral vectors, or helper-dependent adenoviral (HDAd) vectors) are engineered to remove all viral coding sequences, but retain the ITRs of the genome and a packaging sequence of the genome. HDAd genomes are helper-dependent because they do not encode proteins necessary for viral production: a helper-dependent genome can only be packaged into a vector if they are present in a cell that includes a nucleic acid sequence that provides viral proteins in trans. These helper-dependent vectors are also characterized by still greater capacity and further decreased immunogenicity. By deleting the viral coding sequences and leaving only the cis-acting elements necessary for genome replication (ITRs) and packaging, cellular immune response against the Ad vector is reduced. Helper-dependent adenoviral vectors (HDAd) engineered to lack all viral coding sequences can efficiently transduce a wide variety of cell types, and can mediate long-term transgene expression with negligible chronic toxicity. HDAd vectors have a large cloning capacity of up to, e.g., 37 kb, allowing for the delivery of large payloads. These payloads can include large therapeutic genes or even multiple transgenes and large regulatory components to enhance, prolong, and regulate transgene expression. In various embodiments, retained portions of the reference genome can be identical in sequence to corresponding sequences of a reference genome or can have less than 100% identity with a reference genome, e.g., at least 99%, 98%, 97%, 96%, 95%, 90%, 85%, 80%, or 75% identity. Accordingly, as will be appreciated by those of skill in the art, a helper-dependent genome includes 5′ and 3′ ITRs of a particular same serotype, a packaging sequence positioned in the 5′ portion of the helper-dependent adenoviral genome with the 5′ ITR (e.g. within 1 kb of, within 500 bp of, within 250 bp of, within 100 bp of, within 50 bp of, or adjacent to the 5′ ITR), a nucleic acid payload (also referred to herein as a payload), and optionally a stuffer

Various serotype genomes and exemplary components such as ITRs and packaging sequences are exemplified by and can be selected from those described elsewhere herein, excluding conditional forms of packaging sequences. For example, ITRs of a helper-dependent adenoviral genome can be ITRs of an Ad3, Ad7, Ad11, Ad14, Ad16, Ad21, Ad34, or Ad50 genome (e.g., a 5′ ITR according to SEQ ID NOs: 101, 119, 137, 155, 173, 191, 209, or 227, and a 3′ ITR according to SEQ ID NOs: 102, 120, 138, 156, 174, 192, 210, or 228), or ITRs that individually and/or together have at least 75% sequence identity (e.g., at least 75%, 80%, 85%, 90%, 95%, 96%, 97%, 98%, 99%, or 100% identity) thereto. For example, a packaging sequence of a helper-dependent adenoviral genome can be a packaging sequence of an Ad3, Ad7, Ad11, Ad14, Ad16, Ad21, Ad34, or Ad50 vector (e.g., a packaging sequence according to SEQ ID NOs: 103, 121, 139, 157, 175, 193, 211, or 229), or a packaging sequence having at least 75% sequence identity (e.g., at least 75%, 80%, 85%, 90%, 95%, 96%, 97%, 98%, 99%, or 100% identity) to the entirety or a portion thereof (e.g., at least 75%, 80%, 85%, 90%, 95%, 96%, 97%, 98%, 99%, or 100% thereof).

As disclosed herein, a nucleic acid payload is an engineered nucleic acid that includes one or more nucleic acid sequences that include, encode, and/or express at least one agent that achieves a desired result (e.g., that contributes to a therapeutic goal). Embodiments of the present disclosure can include a wide variety of nucleic acid payloads. Those of skill in the art will appreciate that any nucleic acid payloads capable of use in a vector of acceptable length can be used in methods and compositions of the present disclosure. Accordingly, nucleic acid payloads of the present disclosure can encode and/or express a wide variety of expression products. A nucleic acid payload can include any of one or more coding sequences that encode one or more expression products, one or more regulatory sequences operably linked to a coding sequence, one or more stuffer sequences, and the like. Without wishing to be bound by any particular nucleic acid payload, exemplary nucleic acid payloads can be engineered in order to achieve a desired result such as a therapeutic effect in a host cell or system, e.g., expression of a protein of therapeutic interest or of expression of a gene editing system, e.g., a CRISPR/Cas system, base editing system, or prime editing system to generate a sequence modification of therapeutic interest, e.g., to correct a nucleic acid lesion.

Nucleic acid payloads of the present disclosure can include a gene. A gene can include not only coding sequences but also regulatory regions such as promoters, enhancers, termination regions, locus control regions (LCRs), termination and polyadenylation signal elements, splicing signal elements, silencers, insulators, and the like. A gene can include introns and other DNA sequences spliced from an expressed mRNA transcript, along with variants resulting from alternative splice sites. Coding sequences can also include alternative synonymous codon usage as compared to a reference sequence, e.g., codon usage modified as compared to a reference in accordance with codon preference of a specific organism or target cell type.

A nucleic acid payload can include a single gene or multiple genes. A payload can include a single coding sequence or a plurality of coding sequences. A payload can include a single regulatory sequence or a plurality of regulatory sequences. A payload can include a plurality of coding sequences where the individual expression products of the coding sequences function together, e.g., as in the case of an editing enzyme and guide RNA of an editing system, or independently, e.g., as two separate proteins that do not directly or indirectly bind. As will be appreciated by those of skill in the art, a payload or payload component (e.g., a coding sequence and/or regulatory sequence) that is not naturally and/or endogenously encoded by a vector, host cell, and/or target cell can be referred to herein as heterologous. A payload expression product (e.g., an editing enzyme or other polypeptide or guide RNA encoded by a nucleic acid payload) that is not naturally and/or endogenously encoded and/or expressed by a vector, host cell, and/or target cell can be referred to herein as heterologous.

A nucleic acid payload of the present disclosure can include one or more sequences that encode and/or express any of a variety of expression products. Exemplary payload expression products include proteins, including without limitation replacement therapy proteins for treatment of diseases or conditions characterized by low expression or activity of a biologically active protein as compared to a reference level. Exemplary expression products include CRISPR/Cas, base editor, and prime editor systems (e.g., for one or more therapeutic purposes such as repairing a genetic lesion or abnormality and/or treating a disease, disorder, or condition). Exemplary expression products include antibodies, CARs, and TCRs. Exemplary expression products include small RNAs. In embodiments in which a nucleic acid sequence encodes one or more therapeutic proteins, a nucleic acid sequence encoding the therapeutic protein may be found in the art and/or readily derived from or generated based on the relevant amino acid sequence. In various embodiments, a coding sequence can be codon optimized for expression in mammalian cells (e.g., human cells). A nucleic acid sequence such as a nucleic acid payload, or a portion thereof that encodes one or more expression products or includes one or more genes, can include one or more restriction enzyme sites at the 5′ and/or 3′ ends as a means for isolating the nucleic acid sequence from a particular nucleic acid context and/or positioning the nucleic acid sequence within another nucleic acid context.

In various embodiments, integration of all or a portion of a nucleic acid payload into a host cell genome is not required in order for delivery to the host cell to produce an intended or target effect, e.g., in certain instances in which the intended or target effect includes editing of the host cell genome by a CRISPR, base editor, or prime editor system. In various embodiments, integration of all or a portion of a nucleic acid payload is required or preferred in order for delivery a nucleic acid payload to produce an intended or target effect, e.g., where expression of a payload-encoded expression product is desired in progeny cells of a transduced target cell. In various embodiments, a nucleic acid payload can include a nucleic acid sequence engineered for integration into a host cell genome (an “integrating fragment”), e.g., by recombination or transposition.

Particular examples of payload expression products include γ-globin, Factor VIII, YC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, a FANC family gene (e.g., FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), soluble CD40, CTLA, Fas L, an antibody (e.g., that specifically binds CD4, CD5, CD7, CD52, IL1, IL2, IL6, TNF, P53, PTPN22, or DRB1*1501/DQB1*0602), an antibody to TCR specifically present on autoreactive T cells, IL4, IL10, IL12, IL13, IL1Ra, sIL1RI, SIL1RII, STNFRI, sTNFRII, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and other payload expression products described herein.

A therapeutic payload expression product can be selected to provide a therapeutically effective response against diseases related to red blood cells and clotting. In particular embodiments, the disease is a hemoglobinopathy like thalassemia, or a sickle cell disease/trait. A payload expression product may be, for example, an expression product that induces or increases production of hemoglobin; induces or increases production of β-globin, γ-globin, or α-globin, or increases the availability of oxygen to cells in the body. A payload expression product can be, for example, HBB or CYB5R3. Exemplary effective treatments may, for example, increase blood cell counts, improve blood cell function, or increase oxygenation of cells in patients. In another particular embodiment, the disease is hemophilia. A payload expression product can be, for example, an expression product that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary payload expression products include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a nucleic acid payload encodes a globin gene, wherein the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful to express in various methods and compositions disclosed herein, e.g., for treatment of a subject in need thereof.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or α-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1-4 relate to α-type globin sequences and references 4-12 relate to β-type globin sequences (including β and γ globin sequences), which sequences are hereby incorporated by reference: (1) GenBank accession no. Z84721 (Mar. 19, 1997); (2) NCBI accession no. NM_000517 (Oct. 31, 2000); (3) Hardison et al., J. Mol. Biol. (1991) 222(2):233-249; (4) A Syllabus of Human Hemoglobin Variants (1996), by Titus et al., published by The Sickle Cell Anemia Foundation in Augusta, Ga. (available online at globin.cse.psu.edu); (5) GenBank accession no. J00179 (Aug. 26, 1993) or U01317.1; (6) Tagle et al., Genomics (1992) 13(3):741-760; (7) Grovsfeld et al., Cell (1987) 51(6):975-985; (8) Li et al., Blood (1999) 93(7):2208-2216; (9) Gorman et al., J. Biol. Chem. (2000) 275(46):35914-35919; (10) Slightom et al., Cell (1980) 21(3):627-638; (11) Fritsch et al., Cell (1980) 19(4):959-972; (12) Marotta et al., J. Biol. Chem. (1977) 252(14):5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS; 92:6728-6732, 1995. In some embodiments, a globin gene encodes a G16D gamma globin variant.

An exemplary amino acid sequence of hemoglobin subunit β is provided, for example, at UniProt accession no. P68871. An exemplary amino acid sequence for β-globin is provided, for example, at NCBI accession no. NP_000509.

Nucleic acid payloads can also encode therapeutic molecules such as checkpoint inhibitor reagents, chimeric antigen receptors (e.g., chimeric antigen receptors specific to one or more cancer antigens), and/or T-cell receptors (e.g., T-cell receptors specific to one or more cancer antigens).

As another example, a payload expression product can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; or Fabry disease. A payload expression product can be, for example, an agent that induces production of an enzyme, or that otherwise causes degradation of mucopolysaccharides in lysosomes. Exemplary payload expression products can include IDUA or iduronidase, IDS, GNS, HGSNAT, SGSH, NAGLU, GUSB, GALNS, GLB1, ARSB, and HYAL1. Therapeutic nucleic acid payloads for lysosomal storage disorders may, for example, encode or induce the production of enzymes responsible for the degradation of various substances in lysosomes; reduce, eliminate, prevent, or delay the swelling in various organs, including the head (e.g., Macrocephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a payload expression product can be can be selected to provide a therapeutically effective response against a hyperproliferative disease. In particular embodiments, the hyperproliferative disease is cancer. A payload expression product can be, for example, a tumor suppressor, an agent that induces apoptosis, an enzyme, a gene or polypeptide encoding an antibody, or polypeptide hormone. Exemplary payload expression products can include (in addition to those listed elsewhere herein) 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, ELA, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

A payload expression product can be, for example, an agent useful for immune reconstitution, fighting infection (e.g., an antigen of an infectious agent, a receptor, a coreceptor, a receptor ligand, or a coreceptor ligand). Exemplary payload expression product can include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. As another example, a payload expression product can be selected to provide a therapeutically effective response against an infectious disease.

In various embodiments, a payload of the present disclosure encodes and/or expresses at least one component, or all components, of a gene editing system. Gene editing systems of the present disclosure include base editing systems, prime editing systems, CRISPR systems, zinc finger nucleases, and TALENs. Certain gene editing systems can include a plurality of components including a gene editing enzyme selected from a CRISPR-associated RNA-guided endonuclease, a base editing enzyme, and a prime editing enzyme, optionally in combination with at least one gRNA. Accordingly, gene editing systems of the present disclosure can include either (i) in the case of a CRISPR system, a CRISPR enzyme that is a CRISPR-associated RNA-guided endonuclease and at least one guide RNA (gRNA), (ii) in the case of a base editing system, a base editing enzyme and at least one gRNA, or (iii) in the case of a prime editing system and at least one prime editing gRNA. In certain embodiments, a gene editing system can include engineered zinc finger nucleases (ZFN). For instance, a ZFN is an artificial endonuclease that consists of a designed zinc finger protein (ZFP) fused to the cleavage domain of the FokI restriction enzyme. A ZFN may be redesigned to cleave new targets by developing ZFPs with new sequence specificities. For genome engineering, a ZFN is targeted to cleave a chosen genomic sequence. The cleavage event induced by the ZFN provokes cellular repair processes that in turn mediate efficient modification of the targeted locus. If the ZFN-induced cleavage event is resolved via non-homologous end joining, this can result in small deletions or insertions, effectively leading to gene knockout. If the break is resolved via a homology-based process in the presence of an investigator-provided donor, small changes or entire transgenes can be transferred, often without selection, into the chromosome, which can be referred to as ‘gene correction’ and ‘gene addition,’ respectively.

In some embodiments a gene editing system (e.g., a CRISPR system, base editing system, or prime editing system) is engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with α-globin polypeptide subunits. Human fetal γ-globin genes (HBG1 and HBG2, two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34(12): 927-940, 2018, which is incorporated herein by reference in its entirety and with respect to mutations that increase expression of γ-globin). Certain such mutations are found in the HBG1 promoter or HBG2 promoter.

In various embodiments, a gene editing system designed to increase expression of γ-globin includes an HBG1/2 promoter-targeted gRNA that is designed to increase expression of γ-globin by modification and/or inactivation of a BCL11A repressor protein binding site. In various embodiments, a gene editing system designed to increase expression of γ-globin includes a bcl11a-targeted gRNA that is designed to increase expression of γ-globin by modification and/or inactivation of the erythroid bcl11a enhancer to reduce BCL11A repressor protein expression in erythroid cells. In various embodiments, a gene editing system designed to increase expression of γ-globin includes a gRNA targeted to cause a loss of function mutation in the gene encoding BCL11A.

Ad3, Ad7, Ad11, Ad14, Ad16, Ad21, Ad34, and Ad50 genomes can include a sequence including a restriction enzyme site. In various embodiments, the restriction enzyme site is an I-SceI site having a nucleotide sequence of 18 base pairs (TAGGGATAACAGGGTAAT) (SEQ ID NO: 29). In various embodiments, a sequence including a restriction enzyme site (e.g., an I-SceI site) can be inserted at a position in a reference Ad3, Ad7, Ad11, Ad14, Ad16, Ad21, Ad34, and Ad50 genome sequence. In various embodiments, a sequence including a restriction enzyme site (e.g., an I-SceI site) can be used in place of a canonical sequence in a reference Ad3, Ad7, Ad11, Ad14, Ad16, Ad21, Ad34, and Ad50 genome sequence. In various embodiments, the canonical sequence is the same number of nucleotides in length as the sequence including the restriction enzyme site (e.g., an I-SceI site). In various embodiments, the canonical sequence is a different number of nucleotides in length (e.g., greater or fewer) compared to the sequence including the restriction enzyme site (e.g., an I-SceI site). In various embodiments, a sequence including a restriction enzyme site (e.g., an I-SceI site) can be used in place of a canonical sequence at positions 483-500 of a reference Ad3 genome sequence. In various embodiments, a sequence including a restriction enzyme site (e.g., an I-SceI site) can be used in place of a canonical sequence at positions 483-500 of a reference Ad7 genome sequence. In various embodiments, a sequence including a restriction enzyme site (e.g., an I-SceI site) can be used in place of a canonical sequence at positions 481-498 of a reference Ad11 genome sequence. In various embodiments, a sequence including a restriction enzyme site (e.g., an I-SceI site) can be used in place of a canonical sequence at positions 481-498 of a reference Ad14 genome sequence. In various embodiments, a sequence including a restriction enzyme site (e.g., an I-SceI site) can be used in place of a canonical sequence at positions 481-498 of a reference Ad16 genome sequence. In various embodiments, a sequence including a restriction enzyme site (e.g., an I-SceI site) can be used in place of a canonical sequence at positions 481-498 of a reference Ad21 genome sequence. In various embodiments, a sequence including a restriction enzyme site (e.g., an I-SceI site) can be used in place of a canonical sequence at positions 481-498 of a reference Ad34 genome sequence. In various embodiments, a sequence including a restriction enzyme site (e.g., an I-SceI site) can be used in place of a canonical sequence at positions 480-497 of a reference Ad50 genome sequence.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes some or all of the proteins (e.g., all of the structural viral proteins) required for vector production but has a conditional defect in its packaging sequence, making the helper genome less likely to be packaged into a vector under certain vector production conditions (e.g., under conditions that, and/or in the presence of an agent that, reduces function of the conditionally defective packaging sequence). Thus, in various embodiments, an HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor genome to be selectively packaged into HDAd vectors produced from structural components expressed from the helper genome that has a conditional packaging defect. In other words, Ad3, Ad7, Ad11, Ad14, Ad16, Ad21, Ad34, and Ad50 helper vectors and genomes can be used for production of Ad3, Ad7, Ad11, Ad14, Ad16, Ad21, Ad34, and Ad50 donor vectors, respectively.

In some HDAd vector systems, a helper genome utilizes a recombinase system (e.g., a Cre/loxP system) for conditional packaging. In certain such HDAd vector systems, a helper genome can include a packaging sequence (e.g., a complete packaging sequence or a functional fragment thereof (e.g., a fragment of the packaging sequence that is sufficient for packaging, required for packaging, or required for efficient packaging of the Ad genome into the capsid)) flanked by recombinase (e.g., loxP) sites so that contact with a corresponding recombinase (e.g., Cre recombinase) excises the packaging sequence from the helper genome by recombinase-mediated (e.g., Cre-mediated) site-specific recombination between the recombinase sites (e.g., loxP sites). The present disclosure includes, among other things, Ad3, Ad7, Ad11, Ad14, Ad16, Ad21, Ad34, and Ad50 helper vectors and genomes that include two recombinase sites that flank a packaging sequence, where the two recombinase sites are sites corresponding to (i.e., for, or acted upon by) the same recombinase. In various embodiments, a packaging sequence refers to the portion of a helper genome positioned between two recombinase sites as set forth herein, e.g., between the positions at which first and second recombinase sites are inserted or positioned in a helper genome.

Similar HDAd production systems have been developed using FLP (e.g., FLPe)/frt site-specific recombination, where FLP-mediated recombination between frt sites flanking the packaging sequence of the helper genome reduces or eliminates packaging of helper genomes in producer cells that express FLP.

Thus, examples of recombinase systems include the Flp/Frt system and Cre/loxP system, as well as others such as the Dre/rox system, the Vika/vox system, and the PhiC31 system. Cre is a site-specific DNA recombinase derived from bacteriophage P1 sequences. The Cre/loxP system is described in, for example, EP 02200009B1. Cre/loxP systems can include both canonical loxP sites and/or canonical Cre recombinase and/or variations of one or both. The recognition site of Cre protein is typically a nucleotide sequence of 34 base pairs (ATAACTTCGTATAATGTATGCTATACGAAGTTAT) (SEQ ID NO: 1), referred to as a loxP site. Variants of the lox recognition site that can be used include: lox2272 (ATAACTTCGTATAAaGTATcCTATACGAAGTTAT) (SEQ ID NO: 2); lox511 (ATAACTTCGTATAATGTATaCTATACGAAGTTAT) (SEQ ID NO: 3); lox66 (ATAACTTCGTATANNNTANNNTATACGAACGGTA) (SEQ ID NO: 4); lox71 (TACCGTTCGTATANNNTANNNTATACGAAGTTAT) (SEQ ID NO: 5); loxM2 (ATAACTTCGTATAAgaaAccaTATACGAAGTTAT) (SEQ ID NO: 6); loxM3 (ATAACTTCGTATAtaaTACCATATACGAAGTTAT) (SEQ ID NO: 7); loxM7 (ATAACTTCGTATAAgaTAGAATATACGAAGTTAT) (SEQ ID NO: 8); loxM11 (ATAACTTCGTATAcgaTAccaTATACGAAGTTAT) (SEQ ID NO: 9); and lox5171 (ATAACTTCGTATAATGTgTaCTATACGAAGTTAT) (SEQ ID NO: 10). Variants of Cre recombinase are also known and included herein as disclosed, for example, in Eroshenko and Church, Mutants of Cre recombinase with improved accuracy, Nature Communications, 4:2509 (2013), which is incorporated herein by reference in its entirety and in particular with respect to variants of Cre recombinase. The VCre/VloxP recombinase system was derived from Vibrio plasmid p0908. The sCre/SloxP system is described, e.g., in WO 2010/143606. The Flp/Frt DNA recombinase system was derived from Saccharomyces cerevisiae. The Flp/Frt system includes the recombinase Flp (flippase) that catalyzes DNA-recombination on its Frt recognition sites. In various embodiments, an Frt site includes the sequence GAAGTTCCTATTCtctagaaaGtATAGGAACTTC (SEQ ID NO: 11). Variant Frt sites are also included herein. For example, Senecoff et al., (1987) showed that most mutations within the FRT sequence cause minimal effects if present within only one of the two sites. Variants of the Flp protein include GenBank accession no. ABD57356.1 and GenBank accession no. ANW61888.1. The Dre/rox system is described, e.g., in U.S. Pat. Nos. 7,422,889 and 7,915,037B2. It generally includes a Dre recombinase derived from Enterobacteria phage D6 and a rox recognition site. The Vika/vox system is described, e.g., in U.S. Pat. No. 10,253,332. The PhiC31 recombinase recognizes the AttB/AttP binding sites. In various embodiments, a recombinase site of the present disclosure is a sequence that has at least 70% sequence identity (e.g., 70%, 75%, 80%, 95%, 90%, or 95% sequence identity with a sequence selected from SEQ ID NOs: 1-11).

An Ad3, Ad7, Ad11, Ad14, Ad16, Ad21, Ad34, or Ad50 packaging sequence can include up to five, six, or seven putative “A” repeats. For example, in various embodiments, an Ad3, Ad7, Ad11, Ad14, Ad16, Ad21, Ad34, or Ad50 packaging sequence can include one or more, or all, of AI, AII, AIII, AIV, AV, and/or AVI. In various embodiments, the present disclosure includes a recombinant Ad3, Ad7, Ad11, Ad14, Ad16, Ad21, Ad34, or Ad50 helper vector or genome that includes a packaging sequence flanked by recombinase sites. In various embodiments, an Ad3, Ad7, Ad11, Ad14, Ad16, Ad21, Ad34, or Ad50 packaging sequence refers to a nucleic acid sequence according to SEQ ID NOs: 103, 121, 139, 157, 175, 193, 211, or 229, as provided in Tables 2-17 or a functional fragment thereof (e.g., a fragment that is sufficient for packaging, required for packaging, or required for efficient packaging of the Ad genome into the capsid) (e.g., such that flanking of the packaging sequence with recombinase sites and excision by recombination of the recombinase sites renders the vector or genome deficient for packaging, e.g., by at least 10% as compared to a reference including the packaging sequence, e.g., by at least 10%, 20%, 30%, 40%, 50%, 60%, 70%, 80%, 90%, 91%, 92%, 93%, 94%, 95%, 96%, 97, 98%, 99%, or 100%, optionally wherein the reference includes the packaging sequence flanked by the recombines sites). In various embodiments, a packaging sequence is considered derived from a given serotype when said packaging sequence has at least 80% sequence identity (e.g., at least 80%, 85%, 90%, 95%, 96%, 97%, 98%, or 99% sequence identity) to a packaging sequence of a reference sequence of the same serotype. In various embodiments, an Ad3, Ad7, Ad11, Ad14, Ad16, Ad21, Ad34, or Ad50 packaging sequence includes at least 80 nucleotides (e.g., at least 80, 90, 100, 110, 120, 130, 140, 150, 160, 170, 180, 190, 200, 225, 250, 275, or 300 nucleotides, e.g., a number of nucleotides having a lower bound of 80, 90, 100, 110, 120, 130, 140, or 150 nucleotides and an upper bound of 150, 160, 170, 180, 190, 200, 225, 250, 275, or 300 nucleotides) having at least 80% sequence identity (e.g., at least 80%, 85%, 90%, 95%, 96%, 97%, 98%, or 99% sequence identity) with a corresponding fragment of a nucleic acid sequence according to SEQ ID NOs: 103, 121, 139, 157, 175, 193, 211, or 229, as provided in Tables 2-17. In various embodiments, an adenoviral genome of the present disclosure includes a packaging sequence derived from the same serotype as the 5′ ITR and 3′ ITR of the genome.

Those of skill in the art will appreciate that the term packaging sequence does not necessarily include all of the packaging elements present in a given vector or genome. For example, a helper genome can include recombinase direct repeats that flank a packaging sequence, where the flanked packaging sequence does not include all of the packaging elements present in the helper genome. Accordingly, in certain embodiments, one or two recombinase direct repeats of a helper genome are positioned within a larger packaging sequence, e.g., such that a larger packaging sequence is rendered noncontiguous by introduction of the one or two recombinase direct repeats. In various embodiments, recombinase direct repeats of a helper genome flank a fragment of the packaging sequence such that excision of the flanked packaging sequence by recombination of the recombinase direct repeats reduces or eliminates (more generally, disrupts) packaging of the helper genome and/or ability of the helper genome to be packaged.

In some embodiments, to prevent generation of replication competent Ad (RCA) as a consequence of homologous recombination between the helper and HDAd donor genomes present in producer cells, a “stuffer” sequence can be positioned or inserted into the E3 region to render any recombinants too large to be packaged and/or efficiently packaged.

In some embodiments, production of helper-dependent adenoviral (HDAd) donor vectors can include transfection of a plasmid including the HDAd donor genome and transduction of a helper vector including a helper genome to the same cell, cells, or population of cells. The helper genome can rescue propagation of the HDAd donor vector such that HDAd donor vector can be produced and isolated. In various embodiments, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence flanked by recombinase sites (e.g., loxP sites). Thus, producer cells can be transfected with the HDAd donor genome and transduced with a helper genome bearing a packaging sequence flanked by recombinase sites (e.g., loxP sites), where the cells express a recombinase (e.g., Cre) corresponding to the recombinase sites such that excision of the packaging sequence renders the helper virus genome deficient for packaging (e.g., unpackageable), but still able to provide all of the necessary trans-acting factors for production of HDAd donor vector including the HDAd donor genome. After excision of the packaging sequence, a helper genome is deficient for packaging (e.g., unpackageable) but still able to trans-complement the replication and packaging of the HDAd donor genome. HDAd vectors including the donor genome (e.g., a donor genome including a therapeutic payload) can be isolated from the producer cells. In general, some contamination of helper vectors and/or helper genomes in HDAd viral vectors and HDAd viral vector formulations can occur and can be tolerated. HDAd donor vectors can be further isolated and/or purified of any helper vectors by physical means. Various protocols are known in the art, e.g., at Palmer et al., 2009 Gene Therapy Protocols. Methods in Molecular Biology, Volume 433. Humana Press; Totowa, NJ: 2009. pp. 33-53.

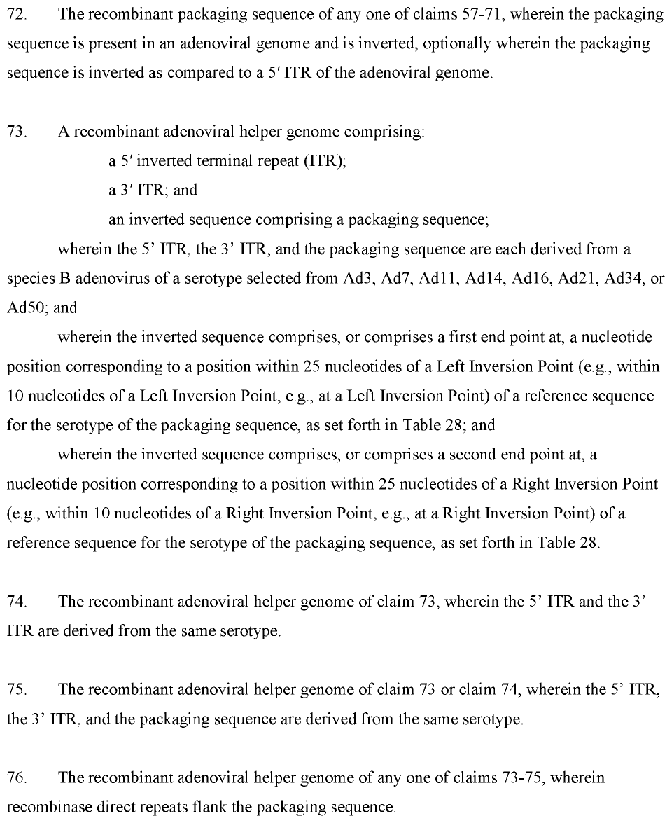

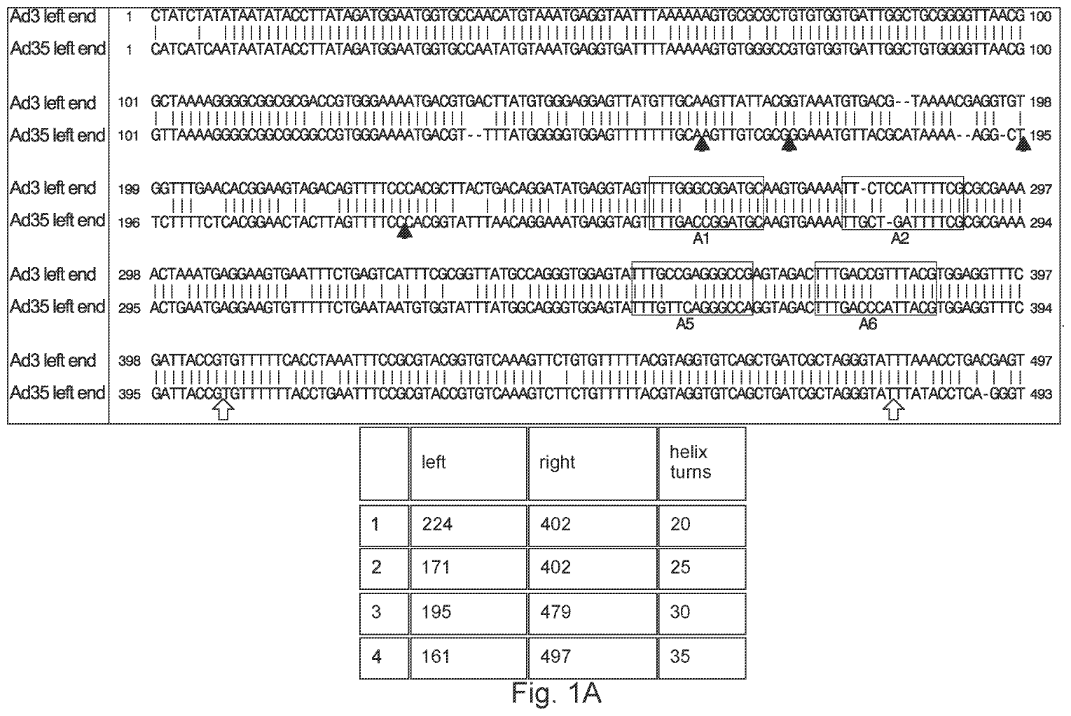

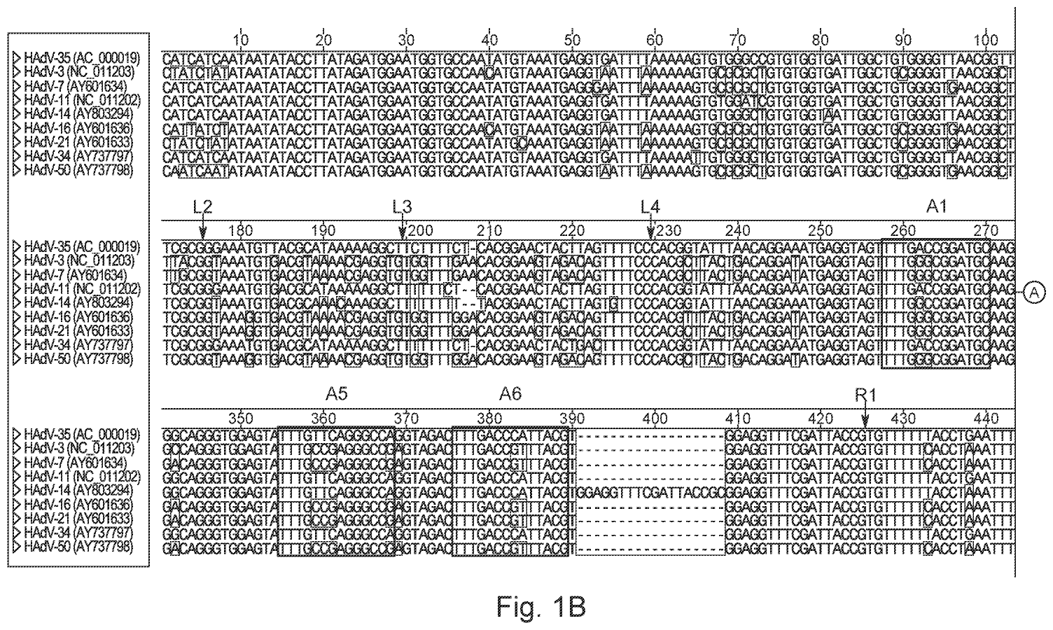

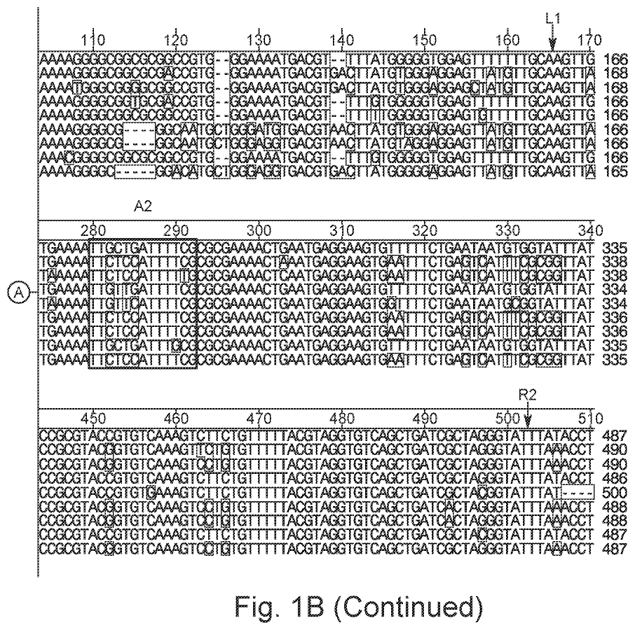

Because the sequences of each viral genome are distinct at least for each serotype, the placement of recombinase sites to produce a helper viral genome cannot be predicted from available information relating to other serotypes. Tables 21-27 include positions for insertion and/or placement of recombinase sites to flank a packaging sequence for use in Ad3, Ad7, Ad11, Ad14, Ad16, Ad21, Ad34, and Ad50 helper genomes, see also FIG. 1B. Four recombinase site positions (L1, L2, L3, and L4) located 5′ (left) of a packaging sequence are provided in relation to reference Ad3, Ad7, Ad11, Ad14, Ad16, Ad21, Ad34, and Ad50 genomes. Three recombinase site positions (R1, R2, and R3) located 3′ (right) of a packaging sequence are provided in relation to reference Ad3, Ad7, Ad11, Ad14, Ad16, Ad21, Ad34, and Ad50 genomes.

TABLE 21
Recombinase site positions for flanking a packaging sequence

| Serotype | L1 | L2 | L3 | L4 | R1 | R2 | R3 | Reference |
|---|---|---|---|---|---|---|---|---|
| Ad35 | 161 | 171 | 195 | 224 | 402 | 479 | 497 | GenBank accession no. |
| | | | | | | | | AY128640 |
| Ad3 | 163 | 173 | 197 | 227 | 405 | 482 | 500 | NCBI accession no. |
| | | | | | | | | NC_011203 |
| Ad7 | 163 | 173 | 197 | 227 | 405 | 482 | 500 | GenBank accession no. |
| | | | | | | | | AY601634 |
| Ad11 | 161 | 171 | 195 | 223 | 401 | 478 | 498 | NCBI accession no. |
| | | | | | | | | NC_011202 |
| Ad14 | 161 | 171 | 195 | 223 | 419 | 496 | 498 | GenBank accession no. |
| | | | | | | | | AY803294 |
| Ad16 | 161 | 171 | 195 | 225 | 403 | 480 | 498 | GenBank accession no. |
| | | | | | | | | AY601636 |
| Ad21 | 161 | 171 | 195 | 225 | 403 | 480 | 498 | GenBank accession no. |
| | | | | | | | | AY601633 |
| Ad34 | 161 | 171 | 195 | 224 | 402 | 479 | 498 | GenBank accession no. |
| | | | | | | | | AY737797 |
| Ad50 | 160 | 170 | 194 | 224 | 402 | 479 | 497 | GenBank accession no. |
| | | | | | | | | AY737798 |

TABLE 22
Recombinase site positions (±1 nt) for flanking a packaging sequence

| Serotype | L1 | L2 | L3 | L4 | R1 | R2 | R3 | Reference |
|---|---|---|---|---|---|---|---|---|
| Ad35 | 160- | 170- | 194- | 223- | 401- | 478- | 496- | GenBank accession no. |
| | 162 | 172 | 196 | 225 | 403 | 480 | 498 | AY128640 |
| Ad3 | 162- | 172- | 196- | 226- | 404- | 481- | 499- | NCBI accession no. |
| | 164 | 174 | 198 | 228 | 406 | 483 | 501 | NC_011203 |
| Ad7 | 162- | 172- | 196- | 226- | 404- | 481- | 499- | GenBank accession no. |
| | 164 | 174 | 198 | 228 | 406 | 483 | 501 | AY601634 |
| Ad11 | 160- | 170- | 194- | 222- | 400- | 477- | 497- | NCBI accession no. |
| | 162 | 172 | 196 | 224 | 402 | 479 | 499 | NC_011202 |
| Ad14 | 160- | 170- | 194- | 222- | 418- | 495- | 497- | GenBank accession no. |
| | 162 | 172 | 196 | 224 | 420 | 497 | 499 | AY803294 |
| Ad16 | 160- | 170- | 194- | 224- | 402- | 479- | 497- | GenBank accession no. |
| | 162 | 172 | 196 | 226 | 404 | 481 | 499 | AY601636 |
| Ad21 | 160- | 170- | 194- | 224- | 402- | 479- | 497- | GenBank accession no. |
| | 162 | 172 | 196 | 226 | 404 | 481 | 499 | AY601633 |
| Ad34 | 160- | 170- | 194- | 223- | 401- | 478- | 497- | GenBank accession no. |
| | 162 | 172 | 196 | 225 | 403 | 480 | 499 | AY737797 |
| Ad50 | 159- | 169- | 193- | 223- | 401- | 478- | 496- | GenBank accession no. |
| | 161 | 171 | 195 | 225 | 403 | 480 | 498 | AY737798 |

TABLE 23
Recombinase site positions (±2 nt) for flanking a packaging sequence

| Serotype | L1 | L2 | L3 | L4 | R1 | R2 | R3 | Reference |
|---|---|---|---|---|---|---|---|---|
| Ad35 | 159- | 169- | 193- | 222- | 400- | 477- | 495- | GenBank accession no. |
| | 163 | 173 | 197 | 226 | 404 | 481 | 499 | AY128640 |
| Ad3 | 161- | 171- | 195- | 225- | 403- | 480- | 498- | NCBI accession no. |
| | 165 | 175 | 199 | 229 | 407 | 484 | 502 | NC_011203 |
| Ad7 | 161- | 171- | 195- | 225- | 403- | 480- | 498- | GenBank accession no. |
| | 165 | 175 | 199 | 229 | 407 | 484 | 502 | AY601634 |
| Ad11 | 159- | 169- | 193- | 221- | 399- | 476- | 496- | NCBI accession no. |
| | 163 | 173 | 197 | 225 | 403 | 480 | 500 | NC_011202 |
| Ad14 | 159- | 169- | 193- | 221- | 417- | 494- | 496- | GenBank accession no. |
| | 163 | 173 | 197 | 225 | 421 | 498 | 500 | AY803294 |
| Ad16 | 159- | 169- | 193- | 223- | 401- | 478- | 496- | GenBank accession no. |
| | 163 | 173 | 197 | 227 | 405 | 482 | 500 | AY601636 |
| Ad21 | 159- | 169- | 193- | 223- | 401- | 478- | 496- | GenBank accession no. |
| | 163 | 173 | 197 | 227 | 405 | 482 | 500 | AY601633 |
| Ad34 | 159- | 169- | 193- | 222- | 400- | 477- | 496- | GenBank accession no. |
| | 163 | 173 | 197 | 226 | 404 | 481 | 500 | AY737797 |
| Ad50 | 158- | 168- | 192- | 222- | 400- | 477- | 495- | GenBank accession no. |
| | 162 | 172 | 196 | 226 | 404 | 481 | 499 | AY737798 |

TABLE 24
Recombinase site positions (±3 nt) for flanking a packaging sequence

| Serotype | L1 | L2 | L3 | L4 | R1 | R2 | R3 | Reference |
|---|---|---|---|---|---|---|---|---|
| Ad35 | 158- | 168- | 192- | 221- | 399- | 476- | 494- | GenBank accession no. |
| | 164 | 174 | 198 | 227 | 405 | 482 | 500 | AY128640 |
| Ad3 | 160- | 170- | 194- | 224- | 402- | 479- | 497- | NCBI accession no. |
| | 166 | 176 | 200 | 230 | 408 | 485 | 503 | NC_011203 |
| Ad7 | 160- | 170- | 194- | 224- | 402- | 479- | 497- | GenBank accession no. |
| | 166 | 176 | 200 | 230 | 408 | 485 | 503 | AY601634 |
| Ad11 | 158- | 168- | 192- | 220- | 398- | 475- | 495- | NCBI accession no. |
| | 164 | 174 | 198 | 226 | 404 | 481 | 501 | NC_011202 |
| Ad14 | 158- | 168- | 192- | 220- | 416- | 493- | 495- | GenBank accession no. |
| | 164 | 174 | 198 | 226 | 422 | 499 | 501 | AY803294 |
| Ad16 | 158- | 168- | 192- | 222- | 400- | 477- | 495- | GenBank accession no. |
| | 164 | 174 | 198 | 228 | 406 | 483 | 501 | AY601636 |
| Ad21 | 158- | 168- | 192- | 222- | 400- | 477- | 495- | GenBank accession no. |
| | 164 | 174 | 198 | 228 | 406 | 483 | 501 | AY601633 |
| Ad34 | 158- | 168- | 192- | 221- | 399- | 476- | 495- | GenBank accession no. |
| | 164 | 174 | 198 | 227 | 405 | 482 | 501 | AY737797 |
| Ad50 | 157- | 167- | 191- | 221- | 399- | 476- | 494- | GenBank accession no. |
| | 163 | 173 | 197 | 227 | 405 | 482 | 500 | AY737798 |

TABLE 25
Recombinase site positions (±4 nt) for flanking a packaging sequence

| Serotype | L1 | L2 | L3 | L4 | R1 | R2 | R3 | Reference |
|---|---|---|---|---|---|---|---|---|
| Ad35 | 157- | 167- | 191- | 220- | 398- | 475- | 493- | GenBank accession no. |
| | 165 | 175 | 199 | 228 | 406 | 483 | 501 | AY128640 |
| Ad3 | 159- | 169- | 193- | 223- | 401- | 478- | 496- | NCBI accession no. |
| | 167 | 177 | 201 | 231 | 409 | 486 | 504 | NC_011203 |
| Ad7 | 159- | 169- | 193- | 223- | 401- | 478- | 496- | GenBank accession no. |
| | 167 | 177 | 201 | 231 | 409 | 486 | 504 | AY601634 |
| Ad11 | 157- | 167- | 191- | 219- | 397- | 474- | 494- | NCBI accession no. |
| | 165 | 175 | 199 | 227 | 405 | 482 | 502 | NC_011202 |
| Ad14 | 157- | 167- | 191- | 219- | 415- | 492- | 494- | GenBank accession no. |
| | 165 | 175 | 199 | 227 | 423 | 500 | 502 | AY803294 |
| Ad16 | 157- | 167- | 191- | 221- | 399- | 476- | 494- | GenBank accession no. |
| | 165 | 175 | 199 | 229 | 407 | 484 | 502 | AY601636 |
| Ad21 | 157- | 167- | 191- | 221- | 399- | 476- | 494- | GenBank accession no. |
| | 165 | 175 | 199 | 229 | 407 | 484 | 502 | AY601633 |
| Ad34 | 157- | 167- | 191- | 220- | 398- | 475- | 494- | GenBank accession no. |
| | 165 | 175 | 199 | 228 | 406 | 483 | 502 | AY737797 |
| Ad50 | 156- | 166- | 190- | 220- | 398- | 475- | 493- | GenBank accession no. |
| | 164 | 174 | 198 | 228 | 406 | 483 | 501 | AY737798 |

TABLE 26
Recombinase site positions (±5 nt) for flanking a packaging sequence

| Serotype | L1 | L2 | L3 | L4 | R1 | R2 | R3 | Reference |
|---|---|---|---|---|---|---|---|---|
| Ad35 | 156- | 166- | 190- | 219- | 397- | 474- | 492- | GenBank accession no. |
| | 166 | 176 | 200 | 229 | 407 | 484 | 502 | AY128640 |
| Ad3 | 158- | 168- | 192- | 222- | 400- | 477- | 495- | NCBI accession no. |
| | 168 | 178 | 202 | 232 | 410 | 487 | 505 | NC_011203 |
| Ad7 | 158- | 168- | 192- | 222- | 400- | 477- | 495- | GenBank accession no. |
| | 168 | 178 | 202 | 232 | 410 | 487 | 505 | AY601634 |
| Ad11 | 156- | 166- | 190- | 218- | 396- | 473- | 493- | NCBI accession no. |
| | 166 | 176 | 200 | 228 | 406 | 483 | 503 | NC_011202 |
| Ad14 | 156- | 166- | 190- | 218- | 414- | 491- | 493- | GenBank accession no. |
| | 166 | 176 | 200 | 228 | 424 | 501 | 503 | AY803294 |
| Ad16 | 156- | 166- | 190- | 220- | 398- | 475- | 493- | GenBank accession no. |
| | 166 | 176 | 200 | 230 | 408 | 485 | 503 | AY601636 |
| Ad21 | 156- | 166- | 190- | 220- | 398- | 475- | 493- | GenBank accession no. |
| | 166 | 176 | 200 | 230 | 408 | 485 | 503 | AY601633 |
| Ad34 | 156- | 166- | 190- | 219- | 397- | 474- | 493- | GenBank accession no. |
| | 166 | 176 | 200 | 229 | 407 | 484 | 503 | AY737797 |
| Ad50 | 155- | 165- | 189- | 219- | 397- | 474- | 492- | GenBank accession no. |
| | 165 | 175 | 199 | 229 | 407 | 484 | 502 | AY737798 |

TABLE 27
Recombinase site positions (±10 nt) for flanking a packaging sequence

| Serotype | L1 | L2 | L3 | L4 | R1 | R2 | R3 | Reference |
|---|---|---|---|---|---|---|---|---|
| Ad35 | 151- | 161- | 185- | 214- | 392- | 469- | 487- | GenBank accession no. |
| | 171 | 181 | 205 | 234 | 412 | 489 | 507 | AY128640 |
| Ad3 | 153- | 163- | 187- | 217- | 395- | 472- | 490- | NCBI accession no. |
| | 173 | 183 | 207 | 237 | 415 | 492 | 510 | NC_011203 |
| Ad7 | 153- | 163- | 187- | 217- | 395- | 472- | 490- | GenBank accession no. |
| | 173 | 183 | 207 | 237 | 415 | 492 | 510 | AY601634 |
| Ad11 | 151- | 161- | 185- | 213- | 391- | 468- | 488- | NCBI accession no. |
| | 171 | 181 | 205 | 233 | 411 | 488 | 508 | NC_011202 |
| Ad14 | 151- | 161- | 185- | 213- | 409- | 486- | 488- | GenBank accession no. |
| | 171 | 181 | 205 | 233 | 429 | 506 | 508 | AY803294 |
| Ad16 | 151- | 161- | 185- | 215- | 393- | 470- | 488- | GenBank accession no. |
| | 171 | 181 | 205 | 235 | 413 | 490 | 508 | AY601636 |
| Ad21 | 151- | 161- | 185- | 215- | 393- | 470- | 488- | GenBank accession no. |
| | 171 | 181 | 205 | 235 | 413 | 490 | 508 | AY601633 |
| Ad34 | 151- | 161- | 185- | 214- | 392- | 469- | 488- | GenBank accession no. |
| | 171 | 181 | 205 | 234 | 412 | 489 | 508 | AY737797 |
| Ad50 | 150- | 160- | 184- | 214- | 392- | 469- | 487- | GenBank accession no. |
| | 170 | 180 | 204 | 234 | 412 | 489 | 507 | AY737798 |

In various embodiments, a first recombinase site (e.g., a loxP site) and a second recombinase site (e.g., a loxP site) are positioned or inserted at positions separated by 178 nucleotides. In various embodiments, a first recombinase site (e.g., a loxP site) and a second recombinase site (e.g., a loxP site) are positioned or inserted at positions separated by 231 nucleotides. In various embodiments, a first recombinase site (e.g., a loxP site) and a second recombinase site (e.g., a loxP site) are positioned or inserted at positions separated by 284 nucleotides. In various embodiments, a first recombinase site (e.g., a loxP site) and a second recombinase site (e.g., a loxP site) are positioned or inserted at positions separated by 337 nucleotides.

In various embodiments, a first recombinase site (e.g., a loxP site) is positioned or inserted at a position corresponding to a position that is 178 nucleotides to the left of a R1 site of a reference genome, as set forth in any one of Tables 21-27. In various embodiments, a first recombinase site (e.g., a loxP site) is positioned or inserted at a position corresponding to a position that is 231 nucleotides to the left of a R1 site of a reference genome, as set forth in any one of Tables 21-27. In various embodiments, a first recombinase site (e.g., a loxP site) is positioned or inserted at a position corresponding to a position that is 284 nucleotides to the left of a R2 site of a reference genome, as set forth in any one of Tables 21-27. In various embodiments, a first recombinase site (e.g., a loxP site) is positioned or inserted at a position corresponding to a position that is 337 nucleotides to the left of a R3 site of a reference genome, as set forth in any one of Tables 21-27.

In various embodiments, a second recombinase site (e.g., a loxP site) is positioned or inserted at a position corresponding to a position that is 337 nucleotides to the right of a L1 site of a reference genome, as set forth in any one of Tables 21-27. In various embodiments, a second recombinase site (e.g., a loxP site) is positioned or inserted at a position corresponding to a position that is 231 nucleotides to the right of a L2 site of a reference genome, as set forth in any one of Tables 21-27. In various embodiments, a second recombinase site (e.g., a loxP site) is positioned or inserted at a position corresponding to a position that is 284 nucleotides to the right of a L3 site of a reference genome, as set forth in any one of Tables 21-27. In various embodiments, a second recombinase site (e.g., a loxP site) is positioned or inserted at a position corresponding to a position that is 178 nucleotides to the right of a L4 site of a reference genome, as set forth in any one of Tables 21-27.

In various embodiments, an Ad3, Ad7, Ad11, Ad14, Ad16, Ad21, Ad34, or Ad50 helper vector can include recombinase sites positioned or inserted to flank a packaging sequence, where a first recombinase site (e.g., a loxP site) is positioned or inserted at (e.g., immediately adjacent to, e.g., before or after) a position corresponding to a position that is within 10 nucleotides of an L1, L2, L3, or L4 site of a reference genome, as set forth in Table 21, and a second recombinase site (e.g., a loxP site) is positioned or inserted at (e.g., immediately adjacent to, e.g., before or after) a position corresponding to a position that is within 10 nucleotides of an R1, R2, or R3 site of a reference genome, as set forth in Table 21.

In certain exemplary embodiments, a first recombinase site (e.g., a loxP site) is positioned or inserted at a position corresponding to a position that is within 10 nucleotides of an L1 site of a reference genome, as set forth in Table 21, and a second recombinase site (e.g., a loxP site) is positioned or inserted at a position corresponding to a position that is within 10 nucleotides of an R3 site of a reference genome, as set forth in Table 21.

In certain exemplary embodiments, a first recombinase site (e.g., a loxP site) is positioned or inserted at a position corresponding to a position that is within 10 nucleotides of an L2 site of a reference genome, as set forth in Table 21, and a second recombinase site (e.g., a loxP site) is positioned or inserted at a position corresponding to a position that is within 10 nucleotides of an R1 site of a reference genome, as set forth in Table 21.

In certain exemplary embodiments, a first recombinase site (e.g., a loxP site) is positioned or inserted at a position corresponding to a position that is within 10 nucleotides of an L3 site of a reference genome, as set forth in Table 21, and a second recombinase site (e.g., a loxP site) is positioned or inserted at a position corresponding to a position that is within 10 nucleotides of an R2 site of a reference genome, as set forth in Table 21.

In certain exemplary embodiments, a first recombinase site (e.g., a loxP site) is positioned or inserted at a position corresponding to a position that is within 10 nucleotides of an L4 site of a reference genome, as set forth in Table 21, and a second recombinase site (e.g., a loxP site) is positioned or inserted at a position corresponding to a position that is within 10 nucleotides of an R1 site of a reference genome, as set forth in Table 21.

In various embodiments, an Ad3, Ad7, Ad11, Ad14, Ad16, Ad21, Ad34, or Ad50 helper vector can include recombinase sites positioned or inserted to flank a packaging sequence, where a first recombinase site (e.g., a loxP site) is positioned or inserted at (e.g., immediately adjacent to, e.g., before or after) a position corresponding to a position at an L1, L2, L3, or L4 site of a reference genome, as set forth in Table 21, and a second recombinase site (e.g., a loxP site) is positioned or inserted at (e.g., immediately adjacent to, e.g., before or after) a position corresponding to a position at an R1, R2, or R3 site of a reference genome, as set forth in Table 21.

In certain exemplary embodiments, a first recombinase site (e.g., a loxP site) is positioned or inserted at a position corresponding to a position at an L1 site of a reference genome, as set forth in Table 21, and a second recombinase site (e.g., a loxP site) is positioned or inserted at a position corresponding to a position at an R3 site of a reference genome, as set forth in Table 21.

In certain exemplary embodiments, a first recombinase site (e.g., a loxP site) is positioned or inserted at a position corresponding to a position at an L2 site of a reference genome, as set forth in Table 21, and a second recombinase site (e.g., a loxP site) is positioned or inserted at a position corresponding to a position at an R1 site of a reference genome, as set forth in Table 21.

In certain exemplary embodiments, a first recombinase site (e.g., a loxP site) is positioned or inserted at a position corresponding to a position at an L3 site of a reference genome, as set forth in Table 21, and a second recombinase site (e.g., a loxP site) is positioned or inserted at a position corresponding to a position at an R2 site of a reference genome, as set forth in Table 21.

In certain exemplary embodiments, a first recombinase site (e.g., a loxP site) is positioned or inserted at a position corresponding to a position at an L4 site of a reference genome, as set forth in Table 21, and a second recombinase site (e.g., a loxP site) is positioned or inserted at a position corresponding to a position at an R1 site of a reference genome, as set forth in Table 21.

In various embodiments, an Ad3, Ad7, Ad11, Ad14, Ad16, Ad21, Ad34, or Ad50 helper vector can include recombinase sites positioned or inserted to flank a packaging sequence, where a first recombinase site (e.g., a loxP site) is positioned or inserted at (e.g., immediately adjacent to, e.g., before or after) a position corresponding to a position at an L1, L2, L3, or L4 site of a reference genome, as set forth in any one of Tables 22-27, and a second recombinase site (e.g., a loxP site) is positioned or inserted at (e.g., immediately adjacent to, e.g., before or after) a position corresponding to a position at an R1, R2, or R3 site of a reference genome, as set forth in any one of Tables 22-27.

In certain exemplary embodiments, a first recombinase site (e.g., a loxP site) is positioned or inserted at a position corresponding to a position at an L1 site of a reference genome, as set forth in any one of Tables 22-27, and a second recombinase site (e.g., a loxP site) is positioned or inserted at a position corresponding to a position at an R3 site of a reference genome, as set forth in any one of Tables 22-27.

In certain exemplary embodiments, a first recombinase site (e.g., a loxP site) is positioned or inserted at a position corresponding to a position at an L2 site of a reference genome, as set forth in any one of Tables 22-27, and a second recombinase site (e.g., a loxP site) is positioned or inserted at a position corresponding to a position at an R1 site of a reference genome, as set forth in any one of Tables 22-27.

In certain exemplary embodiments, a first recombinase site (e.g., a loxP site) is positioned or inserted at a position corresponding to a position at an L3 site of a reference genome, as set forth in any one of Tables 22-27, and a second recombinase site (e.g., a loxP site) is positioned or inserted at a position corresponding to a position at an R2 site of a reference genome, as set forth in any one of Tables 22-27.

In certain exemplary embodiments, a first recombinase site (e.g., a loxP site) is positioned or inserted at a position corresponding to a position at an L4 site of a reference genome, as set forth in any one of Tables 22-27, and a second recombinase site (e.g., a loxP site) is positioned or inserted at a position corresponding to a position at an RI site of a reference genome, as set forth in any one of Tables 22-27.

In certain exemplary embodiments, a first recombinase site (e.g., a loxP site) is positioned or inserted at a position corresponding to a position that is within 10 nucleotides of an L1 site of a reference genome, as set forth in Table 21, and a second recombinase site (e.g., a loxP site) is positioned or inserted at a position corresponding to a position that is within 10 nucleotides of an R1 site of a reference genome, as set forth in Table 21.

In certain exemplary embodiments, a first recombinase site (e.g., a loxP site) is positioned or inserted at a position corresponding to a position that is within 10 nucleotides of an L1 site of a reference genome, as set forth in Table 21, and a second recombinase site (e.g., a loxP site) is positioned or inserted at a position corresponding to a position that is within 10 nucleotides of an R2 site of a reference genome, as set forth in Table 21.

In certain exemplary embodiments, a first recombinase site (e.g., a loxP site) is positioned or inserted at a position corresponding to a position that is within 10 nucleotides of an L2 site of a reference genome, as set forth in Table 21, and a second recombinase site (e.g., a loxP site) is positioned or inserted at a position corresponding to a position that is within 10 nucleotides of an R2 site of a reference genome, as set forth in Table 21.

In certain exemplary embodiments, a first recombinase site (e.g., a loxP site) is positioned or inserted at a position corresponding to a position that is within 10 nucleotides of an L2 site of a reference genome, as set forth in Table 21, and a second recombinase site (e.g., a loxP site) is positioned or inserted at a position corresponding to a position that is within 10 nucleotides of an R3 site of a reference genome, as set forth in Table 21.

In certain exemplary embodiments, a first recombinase site (e.g., a loxP site) is positioned or inserted at a position corresponding to a position that is within 10 nucleotides of an L3 site of a reference genome, as set forth in Table 21, and a second recombinase site (e.g., a loxP site) is positioned or inserted at a position corresponding to a position that is within 10 nucleotides of an R1 site of a reference genome, as set forth in Table 21.

In certain exemplary embodiments, a first recombinase site (e.g., a loxP site) is positioned or inserted at a position corresponding to a position that is within 10 nucleotides of an L3 site of a reference genome, as set forth in Table 21, and a second recombinase site (e.g., a loxP site) is positioned or inserted at a position corresponding to a position that is within 10 nucleotides of an R3 site of a reference genome, as set forth in Table 21.

In certain exemplary embodiments, a first recombinase site (e.g., a loxP site) is positioned or inserted at a position corresponding to a position that is within 10 nucleotides of an L4 site of a reference genome, as set forth in Table 21, and a second recombinase site (e.g., a loxP site) is positioned or inserted at a position corresponding to a position that is within 10 nucleotides of an R2 site of a reference genome, as set forth in Table 21.

In certain exemplary embodiments, a first recombinase site (e.g., a loxP site) is positioned or inserted at a position corresponding to a position that is within 10 nucleotides of an L4 site of a reference genome, as set forth in Table 21, and a second recombinase site (e.g., a loxP site) is positioned or inserted at a position corresponding to a position that is within 10 nucleotides of an R3 site of a reference genome, as set forth in Table 21.

In certain exemplary embodiments, a first recombinase site (e.g., a loxP site) is positioned or inserted at a position corresponding to a position at an L1 site of a reference genome, as set forth in Table 21, and a second recombinase site (e.g., a loxP site) is positioned or inserted at a position corresponding to a position at an R1 site of a reference genome, as set forth in Table 21.

In certain exemplary embodiments, a first recombinase site (e.g., a loxP site) is positioned or inserted at a position corresponding to a position at an L1 site of a reference genome, as set forth in Table 21, and a second recombinase site (e.g., a loxP site) is positioned or inserted at a position corresponding to a position at an R2 site of a reference genome, as set forth in Table 21.

In certain exemplary embodiments, a first recombinase site (e.g., a loxP site) is positioned or inserted at a position corresponding to a position at an L2 site of a reference genome, as set forth in Table 21, and a second recombinase site (e.g., a loxP site) is positioned or inserted at a position corresponding to a position at an R2 site of a reference genome, as set forth in Table 21.

In certain exemplary embodiments, a first recombinase site (e.g., a loxP site) is positioned or inserted at a position corresponding to a position at an L2 site of a reference genome, as set forth in Table 21, and a second recombinase site (e.g., a loxP site) is positioned or inserted at a position corresponding to a position at an R3 site of a reference genome, as set forth in Table 21.

In certain exemplary embodiments, a first recombinase site (e.g., a loxP site) is positioned or inserted at a position corresponding to a position at an L3 site of a reference genome, as set forth in Table 21, and a second recombinase site (e.g., a loxP site) is positioned or inserted at a position corresponding to a position at an R1 site of a reference genome, as set forth in Table 21.

In certain exemplary embodiments, a first recombinase site (e.g., a loxP site) is positioned or inserted at a position corresponding to a position at an L3 site of a reference genome, as set forth in Table 21, and a second recombinase site (e.g., a loxP site) is positioned or inserted at a position corresponding to a position at an R3 site of a reference genome, as set forth in Table 21.

In certain exemplary embodiments, a first recombinase site (e.g., a loxP site) is positioned or inserted at a position corresponding to a position at an L4 site of a reference genome, as set forth in Table 21, and a second recombinase site (e.g., a loxP site) is positioned or inserted at a position corresponding to a position at an R2 site of a reference genome, as set forth in Table 21.

In certain exemplary embodiments, a first recombinase site (e.g., a loxP site) is positioned or inserted at a position corresponding to a position at an L4 site of a reference genome, as set forth in Table 21, and a second recombinase site (e.g., a loxP site) is positioned or inserted at a position corresponding to a position at an R3 site of a reference genome, as set forth in Table 21.

In certain exemplary embodiments, a first recombinase site (e.g., a loxP site) is positioned or inserted at a position corresponding to a position at an L1 site of a reference genome, as set forth in any one of Tables 22-27, and a second recombinase site (e.g., a loxP site) is positioned or inserted at a position corresponding to a position at an R1 site of a reference genome, as set forth in any one of Tables 22-27.

In certain exemplary embodiments, a first recombinase site (e.g., a loxP site) is positioned or inserted at a position corresponding to a position at an L1 site of a reference genome, as set forth in any one of Tables 22-27, and a second recombinase site (e.g., a loxP site) is positioned or inserted at a position corresponding to a position at an R2 site of a reference genome, as set forth in any one of Tables 22-27.

In certain exemplary embodiments, a first recombinase site (e.g., a loxP site) is positioned or inserted at a position corresponding to a position at an L2 site of a reference genome, as set forth in any one of Tables 22-27, and a second recombinase site (e.g., a loxP site) is positioned or inserted at a position corresponding to a position at an R2 site of a reference genome, as set forth in any one of Tables 22-27.

In certain exemplary embodiments, a first recombinase site (e.g., a loxP site) is positioned or inserted at a position corresponding to a position at an L2 site of a reference genome, as set forth in any one of Tables 22-27, and a second recombinase site (e.g., a loxP site) is positioned or inserted at a position corresponding to a position at an R3 site of a reference genome, as set forth in any one of Tables 22-27.

In certain exemplary embodiments, a first recombinase site (e.g., a loxP site) is positioned or inserted at a position corresponding to a position at an L3 site of a reference genome, as set forth in any one of Tables 22-27, and a second recombinase site (e.g., a loxP site) is positioned or inserted at a position corresponding to a position at an R1 site of a reference genome, as set forth in any one of Tables 22-27.

In certain exemplary embodiments, a first recombinase site (e.g., a loxP site) is positioned or inserted at a position corresponding to a position at an L3 site of a reference genome, as set forth in any one of Tables 22-27, and a second recombinase site (e.g., a loxP site) is positioned or inserted at a position corresponding to a position at an R3 site of a reference genome, as set forth in any one of Tables 22-27.

In certain exemplary embodiments, a first recombinase site (e.g., a loxP site) is positioned or inserted at a position corresponding to a position at an L4 site of a reference genome, as set forth in any one of Tables 22-27, and a second recombinase site (e.g., a loxP site) is positioned or inserted at a position corresponding to a position at an R2 site of a reference genome, as set forth in any one of Tables 22-27.

In certain exemplary embodiments, a first recombinase site (e.g., a loxP site) is positioned or inserted at a position corresponding to a position at an L4 site of a reference genome, as set forth in any one of Tables 22-27, and a second recombinase site (e.g., a loxP site) is positioned or inserted at a position corresponding to a position at an R3 site of a reference genome, as set forth in any one of Tables 22-27.

In various embodiments, a sequence 5′ or 3′ of a position for insertion and/or placement of a recombinase site is substituted with an alternative sequence (e.g., an alternative sequence that include a restriction site). In some embodiments, the alternative sequence contains the same number of nucleotides compared to sequence that is replaced. In some embodiments, the alternative sequence contains a different number of nucleotides compared to the sequence that is replaced. Those of skill in the art will appreciate that regardless of such substitution, the corresponding positions (e.g., a corresponding position for insertion of a recombinase site) can still be readily understood by comparison to a reference sequence.

In various embodiments, excision of a packaging sequence from an Ad3, Ad7, Ad11, Ad14, Ad16, Ad21, Ad34, or Ad50 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9%, or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type vector of the same serotype under comparable conditions.

As noted above, homologous recombination between a helper genome including a conditionally defective packaging sequence and a donor genome that includes a wild type or reference packaging sequence can eliminate one or more recombinase sites of a conditionally defective packaging sequence, which can result in contamination of produced donor vectors. This is at least in part because two recombinase sites flanking a packaging sequence are required for excision of the packaging sequence. Excision of one or more of the recombinase sites by recombinase site-excising homologous recombination produces a helper genome that, when contacted with a recombinase corresponding to the recombinase sites, is not rendered defective for packaging (referred to herein as a constitutively packageable helper genome). The present disclosure includes the recognition that packaging sequence inversion can reduce recombinase site-excising homologous recombination.

In some embodiments, a helper genome can include a packaging sequence inversion in that a sequence including the recombinase-site flanked packaging sequence of the helper genome is present in an orientation that differs from a wild-type or reference sequence, such as a reference adenoviral genome. As those of skill in the art will appreciate, nucleic acid sequences are ordered between 5′ and 3′ termini for a given strand, and can be present within a nucleic acid context, such as a strand of genomic DNA having a particular 5′ to 3′ sequence. The “orientation” of a nucleic acid sequence fragment present in a nucleic acid context can refer to whether the order of nucleotides in the fragment is the same as in a corresponding fragment of a wild type or reference nucleic acid context (e.g., a genomic sequence that includes a sequence corresponding to the nucleic acid sequence), or inverted in that a complementary sequence running in the opposite direction (a reverse complement of a corresponding wild-type or reference sequence) is present in the nucleic acid context instead. In various embodiments as used herein, the “orientation” of a flanked packaging sequence or other nucleic acid fragment of an adenoviral genome can refer to the order of nucleotides relative to an ITR, e.g., a 5′ ITR or 3′ ITR. Inversion of a sequence comprising a recombinase-flanked packaging sequence can reduce and/or eliminate recombinase site-excising homologous recombination and thereby prevent production of constitutively packageable helper genomes.

The present disclosure therefore includes, among other things, helper vectors and genomes that include two recombinase sites that flank a packaging sequence, where the two recombinase sites correspond to (i.e., are for, or acted upon by) the same recombinase, and where a sequence that includes the flanked packaging sequence is inverted. In various embodiments, an inverted sequence is or includes a packaging sequence, such as a recombinase site-flanked packaging sequence. In various embodiments, an inverted sequence includes a recombinase site-flanked packaging sequence and one or both of the recombinase sites that flank the packaging sequence.

In various embodiments, an inverted sequence includes additional nucleic acids that are not present in a flanked packaging sequence and/or are not present in the recombinase sites that flank the packaging sequence. In various embodiments, one or both of the 5′ and 3′ ends of an inverted sequence include at least 1, 2, 3, 4, 5, 10, 20, 30, 40, 50, 75, 100, 150, 200, 250, 300, 350, 400, 450, 500, 1,000, or more nucleotides adjacent to a recombinase site. In various embodiments, an inverted sequence includes a number of nucleotides 5′ of a 5′ recombinase site of a flanked packaging sequence that has a lower bound of 1, 2, 3, 4, 5, 10, 20, 30, 40, 50, 75, 100, 150, 200, or 250 nucleotides and an upper bound of 10, 20, 30, 40, 50, 75, 100, 150, 200, 250, 300, 350, 400, 450, 500, or more nucleotides. In various embodiments, an inverted sequence includes a number of nucleotides 3′ of a 3′ recombinase site of a flanked packaging sequence that has a lower bound of 1, 2, 3, 4, 5, 10, 20, 30, 40, 50, 75, 100, 150, 200, or 250 nucleotides and an upper bound of 10, 20, 30, 40, 50, 75, 100, 150, 200, 250, 300, 350, 400, 450, 500, 1,000, or more nucleotides.

In various embodiments, an inverted sequence includes a gene. In various embodiments, an inverted sequence includes E1 or encompasses an E1 deletion. In various embodiments, an inverted sequence includes a gene encoding protein IX. In various embodiments, an inverted sequence includes a gene encoding protein IVa2.

In various embodiments, an inverted sequence does not include an ITR (e.g., a 5′ ITR). In various embodiments, an inverted sequence does not include an exon and/or does not include a portion of an exon. In various embodiments, an inverted sequence is present in a viral genome at a position that does not correspond to its position in a reference or wild-type genome. In various embodiments, an inverted sequence does not include any portion of an E1 coding sequence, protein IX coding sequence, and/or protein IVa2 coding sequence.

In various embodiments, an inverted packaging sequence can include a recombinase-flanked packaging sequence according to any embodiment (e.g., including any recombinase site positions) provided herein. In various embodiments, one or more recombinase sites are positioned within an inverted packaging sequence. In various embodiments, one or more recombinase sites are positioned outside of an inverted packaging sequence. In various embodiments, as provided herein, nucleic acid positions of an adenoviral vector of the present disclosure can be numbered according to a reference disclosed herein. In various embodiments, an inverted packaging sequence can include one or more, or all, of AI, AII, AIII, AIV, AV, and/or AVI, optionally wherein one or more, or all, of AI, AII, AIII, AIV, AV, and/or AVI are present within a sequence flanked by recombinase sites.

In various embodiments, an inverted sequence can be denoted by a first end point and a second end point, where both endpoints correspond to positions within a reference sequence. Tables 28-35 include positions of exemplary first end points (each a Left Inversion Point) and second end points (each a Right Inversion Point) for use in generating an inverted packaging sequence in reference Ad3, Ad7, Ad11, Ad14, Ad16, Ad21, Ad34, and Ad50 genomes. In various embodiments, a Left Inversion Point is not within an ITR (e.g., a 5′ ITR). In various embodiments, ranges for a Left Inversion Point (e.g., as disclosed in Tables 29-35) that encompass an ITR (e.g., a 5′ ITR) can be understood as optionally excluding any portion of the range that overlaps with an ITR disclosed herein. In various embodiments, a Left Inversion Point is within a range according to any one of Tables 29-35 except that the range only includes positions that are greater than or equal to a corresponding Left Inversion Point according to Table 28. In various embodiments, a Right Inversion Point is within a range according to any one of Tables 29-35 except that the range only includes positions that are less than or equal to a corresponding Right Inversion Point according to Table 28.

TABLE 28
End point positions for inverted packaging sequences

| | Left | Right | |
|---|---|---|---|
| | Inversion | Inversion | |
| Serotype | Point | Point | Reference |
| Ad35 | 138 | 497 | GenBank accession no. AY128640 |
| Ad3 | 137 | 500 | NCBI accession no. NC_011203 |
| Ad7 | 137 | 500 | GenBank accession no. AY601634 |
| Ad11 | 138 | 498 | NCBI accession no. NC_011202 |
| Ad14 | 138 | 498 | GenBank accession no. AY803294 |
| Ad16 | 115 | 498 | GenBank accession no. AY601636 |
| Ad21 | 115 | 498 | GenBank accession no. AY601633 |
| Ad34 | 138 | 498 | GenBank accession no. AY737797 |
| Ad50 | 115 | 497 | GenBank accession no. AY737798 |

TABLE 29
End point positions (±1 nt) for inverted packaging sequences

| | Left | Right | |
|---|---|---|---|
| | Inversion | Inversion | |
| Serotype | Point | Point | Reference |
| Ad35 | 137-139 | 496-498 | GenBank accession no. AY128640 |
| Ad3 | 136-138 | 499-501 | NCBI accession no. NC_011203 |
| Ad7 | 136-138 | 499-501 | GenBank accession no. AY601634 |
| Ad11 | 137-139 | 497-499 | NCBI accession no. NC_011202 |
| Ad14 | 137-139 | 497-499 | GenBank accession no. AY803294 |
| Ad16 | 114-116 | 497-499 | GenBank accession no. AY601636 |
| Ad21 | 114-116 | 497-499 | GenBank accession no. AY601633 |
| Ad34 | 137-139 | 497-499 | GenBank accession no. AY737797 |
| Ad50 | 114-116 | 496-498 | GenBank accession no. AY737798 |
| Ad35 | 138-139 | 496-497 | GenBank accession no. AY128640 |
| Ad3 | 137-138 | 499-500 | NCBI accession no. NC_011203 |
| Ad7 | 137-138 | 499-500 | GenBank accession no. AY601634 |
| Ad11 | 138-139 | 497-498 | NCBI accession no. NC_011202 |
| Ad14 | 138-139 | 497-498 | GenBank accession no. AY803294 |
| Ad16 | 115-116 | 497-498 | GenBank accession no. AY601636 |
| Ad21 | 115-116 | 497-498 | GenBank accession no. AY601633 |
| Ad34 | 138-139 | 497-498 | GenBank accession no. AY737797 |
| Ad50 | 115-116 | 496-497 | GenBank accession no. AY737798 |

TABLE 30
End point positions (±2 nt) for inverted packaging sequences

| | Left | Right | |
|---|---|---|---|
| | Inversion | Inversion | |
| Serotype | Point | Point | Reference |
| Ad35 | 136-140 | 495-499 | GenBank accession no. AY128640 |
| Ad3 | 135-139 | 498-502 | NCBI accession no. NC_011203 |
| Ad7 | 135-139 | 498-502 | GenBank accession no. AY601634 |
| Ad11 | 136-140 | 496-500 | NCBI accession no. NC_011202 |
| Ad14 | 136-140 | 496-500 | GenBank accession no. AY803294 |
| Ad16 | 113-117 | 496-500 | GenBank accession no. AY601636 |
| Ad21 | 113-117 | 496-500 | GenBank accession no. AY601633 |
| Ad34 | 136-140 | 496-500 | GenBank accession no. AY737797 |
| Ad50 | 113-117 | 495-499 | GenBank accession no. AY737798 |
| Ad35 | 138-140 | 495-497 | GenBank accession no. AY128640 |
| Ad3 | 137-139 | 498-500 | NCBI accession no. NC_011203 |
| Ad7 | 137-139 | 498-500 | GenBank accession no. AY601634 |
| Ad11 | 138-140 | 496-498 | NCBI accession no. NC_011202 |
| Ad14 | 138-140 | 496-498 | GenBank accession no. AY803294 |
| Ad16 | 115-117 | 496-498 | GenBank accession no. AY601636 |
| Ad21 | 115-117 | 496-498 | GenBank accession no. AY601633 |
| Ad34 | 138-140 | 496-498 | GenBank accession no. AY737797 |
| Ad50 | 115-117 | 495-497 | GenBank accession no. AY737798 |

TABLE 31
End point positions (±3 nt) for inverted packaging sequences

| | Left | Right | |
|---|---|---|---|
| | Inversion | Inversion | |
| Serotype | Point | Point | Reference |
| Ad35 | 135-141 | 494-500 | GenBank accession no. AY128640 |
| Ad3 | 134-140 | 497-503 | NCBI accession no. NC_011203 |
| Ad7 | 134-140 | 497-503 | GenBank accession no. AY601634 |
| Ad11 | 135-141 | 495-501 | NCBI accession no. NC_011202 |
| Ad14 | 135-141 | 495-501 | GenBank accession no. AY803294 |
| Ad16 | 112-118 | 495-501 | GenBank accession no. AY601636 |
| Ad21 | 112-118 | 495-501 | GenBank accession no. AY601633 |
| Ad34 | 135-141 | 495-501 | GenBank accession no. AY737797 |
| Ad50 | 112-118 | 494-500 | GenBank accession no. AY737798 |
| Ad35 | 138-141 | 494-497 | GenBank accession no. AY128640 |
| Ad3 | 137-140 | 497-500 | NCBI accession no. NC_011203 |
| Ad7 | 137-140 | 497-500 | GenBank accession no. AY601634 |
| Ad11 | 138-141 | 495-498 | NCBI accession no. NC_011202 |
| Ad14 | 138-141 | 495-498 | GenBank accession no. AY803294 |
| Ad16 | 115-118 | 495-498 | GenBank accession no. AY601636 |
| Ad21 | 115-118 | 495-498 | GenBank accession no. AY601633 |
| Ad34 | 138-141 | 495-498 | GenBank accession no. AY737797 |
| Ad50 | 115-118 | 494-497 | GenBank accession no. AY737798 |

TABLE 32
End point positions (±4 nt) for inverted packaging sequences

| | Left | Right | |
|---|---|---|---|
| | Inversion | Inversion | |
| Serotype | Point | Point | Reference |
| Ad35 | 134-142 | 493-501 | GenBank accession no. AY128640 |
| Ad3 | 133-141 | 496-504 | NCBI accession no. NC_011203 |
| Ad7 | 133-141 | 496-504 | GenBank accession no. AY601634 |
| Ad11 | 134-142 | 494-502 | NCBI accession no. NC_011202 |
| Ad14 | 134-142 | 494-502 | GenBank accession no. AY803294 |
| Ad16 | 111-119 | 494-502 | GenBank accession no. AY601636 |
| Ad21 | 111-119 | 494-502 | GenBank accession no. AY601633 |
| Ad34 | 134-142 | 494-502 | GenBank accession no. AY737797 |
| Ad50 | 111-119 | 493-501 | GenBank accession no. AY737798 |
| Ad35 | 138-142 | 493-497 | GenBank accession no. AY128640 |
| Ad3 | 137-141 | 496-500 | NCBI accession no. NC_011203 |
| Ad7 | 137-141 | 496-500 | GenBank accession no. AY601634 |
| Ad11 | 138-142 | 494-498 | NCBI accession no. NC_011202 |
| Ad14 | 138-142 | 494-498 | GenBank accession no. AY803294 |
| Ad16 | 115-119 | 494-498 | GenBank accession no. AY601636 |
| Ad21 | 115-119 | 494-498 | GenBank accession no. AY601633 |
| Ad34 | 138-142 | 494-498 | GenBank accession no. AY737797 |
| Ad50 | 115-119 | 493-497 | GenBank accession no. AY737798 |

TABLE 33
End point positions (±5 nt) for inverted packaging sequences

| | Left | Right | |
|---|---|---|---|
| | Inversion | Inversion | |
| Serotype | Point | Point | Reference |
| Ad35 | 133-143 | 492-502 | GenBank accession no. AY128640 |
| Ad3 | 132-142 | 495-505 | NCBI accession no. NC_011203 |
| Ad7 | 132-142 | 495-505 | GenBank accession no. AY601634 |
| Ad11 | 133-143 | 493-503 | NCBI accession no. NC_011202 |
| Ad14 | 133-143 | 493-503 | GenBank accession no. AY803294 |
| Ad16 | 110-120 | 493-503 | GenBank accession no. AY601636 |
| Ad21 | 110-120 | 493-503 | GenBank accession no. AY601633 |
| Ad34 | 133-143 | 493-503 | GenBank accession no. AY737797 |
| Ad50 | 110-120 | 492-502 | GenBank accession no. AY737798 |
| Ad35 | 138-143 | 492-497 | GenBank accession no. AY128640 |
| Ad3 | 137-142 | 495-500 | NCBI accession no. NC_011203 |
| Ad7 | 137-142 | 495-500 | GenBank accession no. AY601634 |
| Ad11 | 138-143 | 493-498 | NCBI accession no. NC_011202 |
| Ad14 | 138-143 | 493-498 | GenBank accession no. AY803294 |
| Ad16 | 115-120 | 493-498 | GenBank accession no. AY601636 |
| Ad21 | 115-120 | 493-498 | GenBank accession no. AY601633 |
| Ad34 | 138-143 | 493-498 | GenBank accession no. AY737797 |
| Ad50 | 115-120 | 492-497 | GenBank accession no. AY737798 |

TABLE 34
End point positions (±10 nt) for inverted packaging sequences

| | Left | Right | |
|---|---|---|---|
| | Inversion | Inversion | |
| Serotype | Point | Point | Reference |
| Ad35 | 128-148 | 487-507 | GenBank accession no. AY128640 |
| Ad3 | 127-147 | 490-510 | NCBI accession no. NC_011203 |
| Ad7 | 127-147 | 490-510 | GenBank accession no. AY601634 |
| Ad11 | 128-148 | 488-508 | NCBI accession no. NC_011202 |
| Ad14 | 128-148 | 488-508 | GenBank accession no. AY803294 |
| Ad16 | 105-125 | 488-508 | GenBank accession no. AY601636 |
| Ad21 | 105-125 | 488-508 | GenBank accession no. AY601633 |
| Ad34 | 128-148 | 488-508 | GenBank accession no. AY737797 |
| Ad50 | 105-125 | 487-507 | GenBank accession no. AY737798 |
| Ad35 | 138-148 | 487-497 | GenBank accession no. AY128640 |
| Ad3 | 137-147 | 490-500 | NCBI accession no. NC_011203 |
| Ad7 | 137-147 | 490-500 | GenBank accession no. AY601634 |
| Ad11 | 138-148 | 488-498 | NCBI accession no. NC_011202 |
| Ad14 | 138-148 | 488-498 | GenBank accession no. AY803294 |
| Ad16 | 115-125 | 488-498 | GenBank accession no. AY601636 |
| Ad21 | 115-125 | 488-498 | GenBank accession no. AY601633 |
| Ad34 | 138-148 | 488-498 | GenBank accession no. AY737797 |
| Ad50 | 115-125 | 487-497 | GenBank accession no. AY737798 |

TABLE 35
End point positions (±25 nt) for inverted packaging sequences

| | Left | Right | |
|---|---|---|---|
| | Inversion | Inversion | |
| Serotype | Point | Point | Reference |
| Ad35 | 113-163 | 472-522 | GenBank accession no. AY128640 |
| Ad3 | 112-162 | 475-525 | NCBI accession no. NC_011203 |
| Ad7 | 112-162 | 475-525 | GenBank accession no. AY601634 |
| Ad11 | 113-163 | 473-523 | NCBI accession no. NC_011202 |
| Ad14 | 113-163 | 473-523 | GenBank accession no. AY803294 |
| Ad16 | 90-140 | 473-523 | GenBank accession no. AY601636 |
| Ad21 | 90-140 | 473-523 | GenBank accession no. AY601633 |
| Ad34 | 113-163 | 473-523 | GenBank accession no. AY737797 |
| Ad50 | 90-140 | 472-522 | GenBank accession no. AY737798 |
| Ad35 | 138-163 | 472-497 | GenBank accession no. AY128640 |
| Ad3 | 137-162 | 475-500 | NCBI accession no. NC_011203 |
| Ad7 | 137-162 | 475-500 | GenBank accession no. AY601634 |
| Ad11 | 138-163 | 473-498 | NCBI accession no. NC_011202 |
| Ad14 | 138-163 | 473-498 | GenBank accession no. AY803294 |
| Ad16 | 115-140 | 473-498 | GenBank accession no. AY601636 |
| Ad21 | 115-140 | 473-498 | GenBank accession no. AY601633 |
| Ad34 | 138-163 | 473-498 | GenBank accession no. AY737797 |
| Ad50 | 115-140 | 472-497 | GenBank accession no. AY737798 |

In some embodiments, the inverted sequence is or includes a sequence corresponding to a portion of a reference sequence that comprises a first end point at a nucleotide position corresponding to a position within 25 nucleotides of a Left Inversion Point of a reference sequence, as set forth in Table 28. In some embodiments, the inverted sequence is or includes a sequence corresponding to a portion of a reference sequence that comprises a second end point at a nucleotide position corresponding to a position within 25 nucleotides of a Right Inversion Point of a reference sequence, as set forth in Table 28.

In some embodiments, the inverted sequence is or includes a sequence corresponding to a portion of a reference sequence that comprises a first end point at a nucleotide position corresponding to a position within 10 nucleotides of a Left Inversion Point of a reference sequence, as set forth in Table 28. In some embodiments, the inverted sequence is or includes a sequence corresponding to a portion of a reference sequence that comprises a second end point at a nucleotide position corresponding to a position within 10 nucleotides of a Right Inversion Point of a reference sequence, as set forth in Table 28.

In some embodiments, the inverted sequence is or includes a sequence corresponding to a portion of a reference sequence that comprises a first end point at a nucleotide position corresponding to a position at a Left Inversion Point of a reference sequence, as set forth in Table 28. In some embodiments, the inverted sequence is or includes a sequence corresponding to a portion of a reference sequence that comprises a second end point at a nucleotide position corresponding to a position at a Right Inversion Point of a reference sequence, as set forth in Table 28.

In some embodiments, the inverted sequence is or includes a sequence corresponding to a portion of a reference sequence that comprises a first end point at a nucleotide position corresponding to a position at a Left Inversion Point of a reference sequence, as set forth in any one of Table 29-35. In some embodiments, the inverted sequence is or includes a sequence corresponding to a portion of a reference sequence that comprises a second end point at a nucleotide position corresponding to a position at a Right Inversion Point of a reference sequence, as set forth in any one of Table 29-35.

In certain exemplary embodiments,
(i) a first recombinase site (e.g., a loxP site) at a nucleotide position corresponding to a position within 10 nucleotides of an L1, L2, L3, or L4 site of a reference sequence, as set forth in Table 21;
(ii) a second recombinase site (e.g., a loxP site) at a nucleotide position corresponding to a position within 10 nucleotides of an R1, R2, or R3 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position within 10 nucleotides of an L1 site of a reference sequence, as set forth in Table 21;
(ii) a second recombinase site (e.g., a loxP site) at a nucleotide position corresponding to a position within 10 nucleotides of an R3 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position within 10 nucleotides of an L2 site of a reference sequence, as set forth in Table 21;
(ii) a second recombinase site (e.g., a loxP site) at a nucleotide corresponding to a position within 10 nucleotides of an RI site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position within 10 nucleotides of an L3 site of a reference sequence, as set forth in Table 21;
(ii) a second recombinase site (e.g., a loxP site) at a nucleotide corresponding to a position within 10 nucleotides of an R2 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position within 10 nucleotides of an L4 site of a reference sequence, as set forth in Table 21;
(ii) a second recombinase site (e.g., a loxP site) at a nucleotide position corresponding to a position within 10 nucleotides of an R1 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L1, L2, L3, or L4 site of a reference sequence, as set forth in Table 21;
(ii) a second recombinase site (e.g., a loxP site) at a nucleotide position corresponding to a position at an R1, R2, or R3 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L1 site of a reference sequence, as set forth in Table 21;
(ii) a second recombinase site (e.g., a loxP site) at a nucleotide position corresponding to a position at an R3 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L2 site of a reference sequence, as set forth in Table 21;
(ii) a second recombinase site (e.g., a loxP site) at a nucleotide position corresponding to a position at an R1 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L3 site of a reference sequence, as set forth in Table 21;
(ii) a second recombinase site (e.g., a loxP site) at a nucleotide position corresponding to a position at an R2 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L4 site of a reference sequence, as set forth in Table 21;
(ii) a second recombinase site (e.g., a loxP site) at a nucleotide position corresponding to a position at an R1 site of a reference sequence, as set forth in Table 21; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L1, L2, L3, or L4 site of a reference sequence, as set forth in any one of Tables 22-27;
(ii) a second recombinase site (e.g., a loxP site) at a nucleotide position corresponding to a position at an R1, R2, or R3 site of a reference sequence, as set forth in any one of Tables 22-27; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in any one of Table 26, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L1 site of a reference sequence, as set forth in any one of Tables 22-27;
(ii) a second recombinase site (e.g., a loxP site) at a nucleotide position corresponding to a position at an R3 site of a reference sequence, as set forth in any one of Tables 22-27; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L2 site of a reference sequence, as set forth in any one of Tables 22-27;
(ii) a second recombinase site (e.g., a loxP site) at a nucleotide position corresponding to a position at an R1 site of a reference sequence, as set forth in any one of Tables 22-27; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L3 site of a reference sequence, as set forth in any one of Tables 22-27;
(ii) a second recombinase site (e.g., a loxP site) at a nucleotide position corresponding to a position at an R2 site of a reference sequence, as set forth in any one of Tables 22-27; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

In certain exemplary embodiments,
(i) a first recombinase site (e.g., a loxP site) at a nucleotide position corresponding to a position at an L4 site of a reference sequence, as set forth in any one of Tables 22-27;
(ii) a second recombinase site (e.g., a loxP site) at a nucleotide position corresponding to a position at an RI site of a reference sequence, as set forth in any one of Tables 22-27; and
(iii) an inversion of a sequence that includes:
   (a) a first end point at a position corresponding to a position within 25 nucleotides of a Left Inversion Point (e.g., within 10 nucleotides of a Left Inversion Point, e.g., at a Left Inversion Point) of a reference sequence, as set forth in Table 28, and
   (b) a second end point at a position corresponding to a position within 25 nucleotides of a Right Inversion Point (e.g., within 10 nucleotides of a Right Inversion Point, e.g., at a Right Inversion Point) of a reference sequence, as set forth in Table 28.

Those of skill in the art will appreciate that there are a variety of means by which an inversion can be produced within a larger sequence such as an adenoviral genome. Such inversions can be produced using various available tools of molecular biology. For example, in certain embodiments, an inverted sequence can by synthesized or isolated and inserted into a target sequence by various means known in the art. In certain embodiments, a sequence for inversion can be positioned between two copies of a palindromic restriction site, such that contacting a sequence including the sequence for inversion flanked by the restriction sites can result in an inversion in accordance with various methods known in the art.

To provide one non-limiting example of a technique for producing an inversion of a sequence within an adenoviral genome, FseI sites can be inserted at positions that flank a packaging sequence or sequence including a packaging sequence, optionally wherein the packaging sequence is flanked by recombinase sites that are in turn between the FseI sites. Such a sequence can be digested with FseI to excise the FseI site-flanked sequence, which excised sequence can then be re-ligated into the digested sequence in the opposite orientation.

The present disclosure includes, among other things, Ad3, Ad7, Ad11, Ad14, Ad16, Ad21, Ad34, and Ad50 helper genomes as disclosed herein and Ad3, Ad7, Ad11, Ad14, Ad16, Ad21, Ad34, and Ad50 helper vectors that include the same. The present disclosure further includes use of Ad3, Ad7, Ad11, Ad14, Ad16, Ad21, Ad34, and Ad50 helper genomes and vectors in a method or composition for production of helper-dependent adenoviral (HDAd) donor vectors of serotype Ad3, Ad7, Ad11, Ad14, Ad16, Ad21, Ad34, or Ad50. The present disclosure further includes cells that include Ad3, Ad7, Ad11, Ad14, Ad16, Ad21, Ad34, or Ad50 helper vectors and/or Ad35 helper genomes (and optionally further include an HDAd donor genome of serotype Ad3, Ad7, Ad11, Ad14, Ad16, Ad21, Ad34, or Ad50), e.g., for production of HDAd35 donor vectors. The present disclosure further includes use of such cells in a method or composition for production of HDAd donor vectors of serotype Ad3, Ad7, Ad11, Ad14, Ad16, Ad21, Ad34, or Ad50. In certain such cells, viral proteins encoded and expressed by the helper genome can be utilized in production of HDAd donor vectors of serotype Ad3, Ad7, Ad11, Ad14, Ad16, Ad21, Ad34, or Ad50 in which the HDAd donor genome of serotype Ad3, Ad7, Ad11, Ad14, Ad16, Ad21, Ad34, or Ad50 is packaged. Accordingly, the present disclosure includes methods of production of HDAd donor vectors of serotype Ad3, Ad7, Ad11, Ad14, Ad16, Ad21, Ad34, or Ad50 by culturing cells that include an HDAd donor genome of serotype Ad3, Ad7, Ad11, Ad14, Ad16, Ad21, Ad34, or Ad50 and an Ad3, Ad7, Ad11, Ad14, Ad16, Ad21, Ad34, or Ad50 helper genome. In some embodiments, the cells encode and express a recombinase that corresponds to recombinase direct repeats that flank a packaging sequence of the Ad3, Ad7, Ad11, Ad14, Ad16, Ad21, Ad34, or Ad50 helper vector. In some embodiments, the flanked packaging sequence of the Ad3, Ad7, Ad11, Ad14, Ad16, Ad21, Ad34, or Ad50 helper genome has been excised.

In various embodiments, an Ad3, Ad7, Ad11, Ad14, Ad16, Ad21, Ad34, or Ad50 helper genome includes a conditional (e.g., frt-site or loxP-site flanked) packaging sequence and encodes all of the necessary proteins for production of Ad3, Ad7, Ad11, Ad14, Ad16, Ad21, Ad34, or Ad50 virions into which a donor genome can be packaged. In some embodiments, the Ad3, Ad7, Ad11, Ad14, Ad16, Ad21, Ad34, or Ad50 helper genome encodes all Ad3, Ad7, Ad11, Ad14, Ad16, Ad21, Ad34, or Ad50 coding sequences, respectively.

An additional optional engineering consideration can be engineering of a helper genome having a size that permits separation of helper vector from HDAd donor vector by centrifugation, e.g., by CsCl ultracentrifugation. One means of achieving this result is to increase the size of the helper genome as compared to a typical adenoviral genome of the same serotype. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure such as a helper genome can include a selection of components each selected from, or having at least 75% sequence identity (e.g., at least 75%, 80%, 85%, 90%, 95%, 96%, 97%, 98%, 99%, or 100% identity) to, a corresponding sequence of a reference genome.

The present disclosure includes systems, kits, and methods for production of helper-dependent adenoviral vectors. Broadly, systems and kits for production of helper-dependent adenoviral vectors can include one or more of a helper adenoviral genome (optionally present in a nucleic acid molecule such as a plasmid or other nucleic acid vector), a helper adenoviral vector, a helper-dependent adenoviral genome (optionally present in a nucleic acid molecule such as a plasmid or other nucleic acid vector), a helper-dependent adenoviral vector, and a production cell.

In certain embodiments, a system, kit, or method for production of helper-dependent adenoviral vectors can include a helper adenoviral genome and a helper-dependent adenoviral genome. In certain embodiments, a system, kit, or method for production of helper-dependent adenoviral vectors can include a helper adenoviral vector and a helper-dependent adenoviral vector. In certain embodiments, a system, kit, or method for production of helper-dependent adenoviral vectors can include a helper adenoviral vector and a helper-dependent adenoviral genome. In certain embodiments, a system, kit, or method for production of helper-dependent adenoviral vectors can include a helper adenoviral genome and a helper-dependent adenoviral vector.

In certain embodiments, a system, kit, or method for production of helper-dependent adenoviral vectors can include a helper adenoviral genome, a helper-dependent adenoviral genome, and a producer cell. In certain embodiments, a system, kit, or method for production of helper-dependent adenoviral vectors can include a helper adenoviral vector, a helper-dependent adenoviral vector, and a producer cell. In certain embodiments, a system, kit, or method for production of helper-dependent adenoviral vectors can include a helper adenoviral vector, a helper-dependent adenoviral genome, and a producer cell. In certain embodiments, a system, kit, or method for production of helper-dependent adenoviral vectors can include a helper adenoviral genome, a helper-dependent adenoviral vector, and a producer cell.

In certain embodiments, a system, kit, or method for production of helper-dependent adenoviral vectors can include a producer cell that includes a helper adenoviral genome and a helper-dependent adenoviral genome. In certain embodiments, a system, kit, or method for production of helper-dependent adenoviral vectors can include a producer cell that includes a helper adenoviral vector and a helper-dependent adenoviral vector. In certain embodiments, a system, kit, or method for production of helper-dependent adenoviral vectors can include a producer cell that includes a helper adenoviral vector and a helper-dependent adenoviral genome. In certain embodiments, a system, kit, or method for production of helper-dependent adenoviral vectors can include a producer cell that includes a helper adenoviral genome and a helper-dependent adenoviral vector.

Those of skill in the art will appreciate from the present disclosure that systems, kits, and methods of the present disclosure can include a helper adenoviral genome or vector and a helper-dependent genome or vector, where the helper genome encodes proteins for production of adenoviral vectors into which the helper-dependent genome can be packaged. For example, in various embodiments, a system, kit, or method of the present disclosure can include a helper adenoviral genome or vector of a particular serotype and a helper-dependent genome that is of the same serotype. In certain particular examples, a system, kit, or method of the present disclosure can include a helper adenoviral genome or vector that encodes ITRs of a particular serotype and a helper-dependent genome that has ITRs of the same serotype. In certain particular examples, a system, kit, or method of the present disclosure can include a helper adenoviral genome or vector that encodes ITRs and a packaging sequence (e.g., a conditional packaging sequence) of a particular serotype and a helper-dependent genome that has ITRs and a packaging sequence of the same serotype. In certain particular examples, a system, kit, or method of the present disclosure can include a helper adenoviral genome or vector that encodes one or more capsid proteins or protein fragments (e.g., one or more of a hexon, penton, fiber, fiber tail, fiber shaft, or fiber knob) of a particular serotype and a helper-dependent genome that has ITRs of the same serotype.

Those of skill in the art will appreciate from the present disclosure that systems, kits, and methods of the present disclosure can include a helper genome or vector that includes one or more of an E1 region deletion, an E3 region deletion, and/or an E4 region deletion. Those of skill in the art will further appreciate from the present disclosure that in various embodiments a producer cell genome encodes and/or expresses one or more adenoviral expression products, or includes adenoviral sequences, that can correspond to the serotype of the helper genome or vector and/or complement the helper genome or vector (e.g., in that proteins encoded and/or expressed by the producer cell genome permit or facilitate production of adenoviral vectors and/or packaging of helper-dependent adenoviral genomes, which are optionally present).

In various embodiments a producer cell genome encodes and/or expresses one or more E1 expression products (e.g., includes an E1 region) that can correspond to the serotype of the helper genome or vector and/or complement the helper genome or vector (e.g., in that proteins encoded and/or expressed by the producer cell genome permit or facilitate production of adenoviral vectors and/or packaging of helper-dependent adenoviral genomes, which are optionally present). In various embodiments a producer cell genome encodes and/or expresses one or more E1 expression products including at least E1b55k (e.g., includes an E1 region encoding at least E1b55k), that can correspond to the serotype of the helper genome or vector and/or complement the helper genome or vector (e.g., in that proteins encoded and/or expressed by the producer cell genome permit or facilitate production of adenoviral vectors and/or packaging of helper-dependent adenoviral genomes, which are optionally present). An E1b55k expression product, or E1 region sequence encoding an E1b55k expression product, can be said to complement an E4orf6 expression product, or E1 region sequence encoding an E4orf6 expression product, if the E1b55k and E4orf6 are able to interact in a manner that promotes, causes, enables, permits, or facilitates production of adenoviral vectors and/or packaging of helper-dependent adenoviral genomes, which helper-dependent adenoviral genomes are optionally present.

Those of skill in the art will further appreciate from the present disclosure that in various embodiments a producer cell genome encodes and/or expresses one or more E1 expression products (e.g., includes an E1 region) that can correspond to the serotype of the helper genome or vector and/or complement the helper genome or vector in that it is of the same serotype as an E4 region of the helper genome or vector, or is of the same serotype as an E4 expression product encoded and/or expressed by the helper genome or vector. In various embodiments, a producer cell genome encodes and/or expresses one or more E1 expression products (e.g., includes an E1 region) that do not correspond to the serotype of the helper genome or vector, but which correspond to the serotype of a heterologous E4 region of the helper genome or vector, or is of the same serotype as a heterologous E4 expression product encoded and/or expressed by the helper genome or vector. E1 and E4 regions and/or expression products can be referred to as complementary where the producer cell genome E1 region and/or expression products and the helper genome E4 region and/or expression products together are necessary or sufficient to promote, cause, enable, permit, or facilitate production of adenoviral vectors and/or packaging of helper-dependent adenoviral genomes, which helper-dependent adenoviral genomes are optionally present.

Those of skill in the art will further appreciate from the present disclosure that in various embodiments a producer cell genome encodes and/or expresses one or more E1 expression products including at least E1b55k (e.g., includes an E1 region encoding at least E1b55k) that can correspond to the serotype of the helper genome or vector and/or complement the helper genome or vector in that it is of the same serotype as an E4 region of the helper genome or vector encoding at least E4orf6, or is of the same serotype as an E4orf6 expression product encoded and/or expressed by the helper genome or vector. In various embodiments a producer cell genome encodes and/or expresses one or more E1 expression products including at least E1b55k (e.g., includes an E1 region encoding at least E1b55k) that do not correspond to the serotype of the helper genome or vector, but which correspond to the serotype of a heterologous E4 region of the helper genome or vector encoding at least E4orf6, or is of the same serotype as a heterologous E4orf6 expression product encoded and/or expressed by the helper genome or vector. E1 and E4 regions and/or expression products can be referred to as complementary where the producer cell genome E1 region encoding at least E1b55k and/or expression products including at least E1b55k and the helper genome E4 region encoding at least E4orf6 together are necessary or sufficient to promote, cause, enable, permit, or facilitate production of adenoviral vectors and/or packaging of helper-dependent adenoviral genomes, which helper-dependent adenoviral genomes are optionally present. In various embodiments, complementary E1 and E4 regions are both present in a producer cell genome, e.g., where a helper vector includes deletions of both E1 and E4 regions. Those of skill in the art will further appreciate that it is necessary for a producer cell genome to encode and/or express an E1 region (e.g., an E1 region encoding E1B55K) where a helper genome encodes and/or expresses E4orf3 (e.g., where the helper genome comprises an E4 deletion that does not delete E4orf3). In an illustrative example, E4 functions can be complemented in producer cells by expression of a complete E4 region, e.g., using an inducible MMTV LTR (mouse mammary tumor virus long terminal repeat) promoter (reference HAdV-5 sequence GenBank accession no. M73260).

Those of skill in the art will further appreciate from the present disclosure that in various embodiments a producer cell genome encodes and/or expresses one or more E2 expression products, e.g., including at least E2 DBP (e.g., includes an E2 region encoding at least E2 DBP) that can correspond to the serotype of the helper genome or vector and/or complement the helper genome or vector. In various embodiments a producer cell genome encodes and/or expresses one or more E2 expression products including at least E2 DBP (e.g., includes an E2 region encoding at least E2 DBP) that correspond to the serotype of the helper genome or vector. An E2 DBP sequence a helper genome can be referred to as complementary where a producer cell genome E2 region encoding at least E2 DBP is sufficient to promote, cause, enable, permit, or facilitate production of adenoviral vectors and/or packaging of helper-dependent adenoviral genomes, which helper-dependent adenoviral genomes are optionally present. Accordingly in various embodiments a producer cell genome can encode and/or express an E1 region, and E2a region, an E3 region, and/or an E4 region, optionally where one or more or all of an E1 region, an E2 region, an E3 region, and an E4 region are deleted from an adenoviral helper genome.

The present disclosure further includes a Ad3, Ad7, Ad11, Ad14, Ad16, Ad21, Ad34, or Ad50 helper-dependent vector production system including a cell including a Ad3, Ad7, Ad11, Ad14, Ad16, Ad21, Ad34, or Ad50 helper-dependent genome and an Ad3, Ad7, Ad11, Ad14, Ad16, Ad21, Ad34, or Ad50 helper genome. In certain such cells, viral proteins encoded and expressed by the helper genome can be utilized in production of Ad3, Ad7, Ad11, Ad14, Ad16, Ad21, Ad34, or Ad50 helper-dependent vectors in which the Ad3, Ad7, Ad11, Ad14, Ad16, Ad21, Ad34, or Ad50 helper-dependent genome is packaged. Accordingly, the present disclosure includes methods of production of Ad3, Ad7, Ad11, Ad14, Ad16, Ad21, Ad34, or Ad50 helper-dependent vectors by culturing cells that include an Ad3, Ad7, Ad11, Ad14, Ad16, Ad21, Ad34, or Ad50 helper-dependent genome and an Ad3, Ad7, Ad11, Ad14, Ad16, Ad21, Ad34, or Ad50 helper genome. In some embodiments the cells encode and express a recombinase that corresponds to recombinase direct repeats that flank a packaging sequence of the Ad3, Ad7, Ad11, Ad14, Ad16, Ad21, Ad34, or Ad50 helper vector. In some embodiments, the flanked packaging sequence of the Ad3, Ad7, Ad11, Ad14, Ad16, Ad21, Ad34, or Ad50 helper genome has been excised.

Various producer cells are known in the art. In various embodiments, a producer cell can be a cell that encodes and/or expresses a recombinase that corresponds to recombination sites present in a conditional packaging sequence of a helper genome. In various embodiments, a producer cell can be a cell that include and/or expresses a heterologous E1 region. In various embodiments, a producer cell can include and/or express an E1 region of an adenoviral serotype such as an Ad5 serotype. In various embodiments, a producer cell is a HEK293 cell (also referred to herein as a 293 cell) that expresses Cre recombinase and includes and/or expresses an Ad5 E1 region. In various embodiments, a producer cell is a 116 cell. In various embodiments, a producer cell is a 911 human embryonic retinoblast cell, a pTG6559 A549 cell, a PER.C6 human embryonic retinoblast cell, a GH329 HeLa cell, an N52.E6 primary human aminocyte cell, a HeLa-E1 cell, an UR HEL 299 cell, or a VLI-293 HEK293 cell. Examples of producer cells known in the art are described, e.g., in Kovesdi 2010 Viruses 2(8):1681-1703.

In various embodiments, the level of recombinase expressed by or present in a producer cell can be engineered, e.g., to achieve an increased and/or target level of recombinase and/or recombinase expression. Examples of such engineering are known in the art, see e.g., Palmer and Ng, Mol Ther. 2003; 8(5):846-852; Ng et al., J Virol. 2002; 76(9):4181-4189; and Gonzalez-Aparicio et al., Gene Ther. 2011; 18(11):1025-1033. In some embodiments, the level of recombinase expressed by or present in a producer cell can be engineered by the expression of a recombinase that is encoded by a helper genome.

Production of helper-dependent adenoviral vectors can include production of vectors in or from a producer cell or population of producer cells that include a helper genome and a helper-dependent genome. In certain embodiments, production of helper-dependent adenoviral vectors can include transfection of a plasmid including the helper-dependent adenoviral genome and a transduction of a helper vector including a helper genome to the same cell, cells, or population of cells. The helper genome can rescue propagation of the helper-dependent adenoviral vector and helper-dependent adenoviral vector can be produced, e.g., at a large scale, and purified. Various protocols are known in the art, e.g., at Palmer et al., 2009 Gene Therapy Protocols. Methods in Molecular Biology, Volume 433. Humana Press; Totowa, NJ: 2009. pp. 33-53.

In an exemplary method for production of helper-dependent adenoviral vectors, a helper-dependent adenoviral genome can be delivered to cells that express a recombinase for excision of the conditional packaging sequence of a helper vector (e.g., 293 cells (HEK293) that expresses Cre recombinase), optionally where the helper-dependent adenoviral genome is delivered to the cells in a non-viral vector form, such as a bacterial plasmid form (e.g., where the helper-dependent adenoviral genome is present in a bacterial plasmid (pHDAd) and optionally liberated by restriction enzyme digestion). The same cells can be transduced with the helper genome including a packaging sequence flanked by recombinase sites (e.g., loxP sites), as described elsewhere herein. Thus, in various embodiments, producer cells can be transfected with the helper-dependent adenoviral genome and transduced with a helper genome bearing a packaging sequence or a functional fragment thereof flanked by recombinase sites (e.g., loxP sites), where the cells express a recombinase (e.g., Cre) corresponding to the recombinase sites such that excision of the packaging sequence or functional fragment thereof renders the helper virus genome deficient for packaging (e.g., unpackageable), but still able to provide all of the necessary trans-acting factors for production of helper-dependent adenoviral vector including the helper-dependent adenoviral genome. In various embodiments, a helper genome and a helper-dependent genome can each be independently delivered to a producer cell by any means known in the art, including without limitation by transfection, infection, or transduction (e.g., by a plasmid including the genome or by a vector including the genome).

Helper-dependent adenoviral vectors including a helper-dependent vector genome including a payload can be purified from producer cells. Helper-dependent adenoviral vectors can be further purified from helper vectors by physical means. In general, some contamination of helper vectors and/or helper genomes in helper-dependent adenoviral viral vectors and helper-dependent adenoviral viral vector formulations can occur and can be tolerated. However, minimizing such contamination and/or increasing purity of helper-dependent vector or genome preparations is a challenge in the art.

Helper-dependent adenoviral vectors present in a composition can be purified (e.g., purified from all a portion of helper vectors present in the composition) according to a variety of means known in the art. Various such means are based at least in part on differential mass or density of helper and helper-dependent adenoviral vectors and/or genomes, or of compositions including the same. In various embodiments, purification can be achieved by use of a density gradient (e.g., in a process of density gradient centrifugation, e.g., density gradient ultracentrifugation).

In certain particular embodiments, an exemplary density gradient that can be used in purifying helper-dependent adenoviral vectors is a cesium chloride (CsCl) gradient. Cesium chloride gradients and their use are known in the art. CsCl salt forms a density gradient when subjected to a strong centrifugal field. When the viruses are centrifuged to equilibrium in a CsCl salt, they are separated from contaminants and collected in bands on the basis of their buoyant densities. In certain particular embodiments, an exemplary density gradient that can be used in purifying helper-dependent adenoviral vectors is an iodixanol gradient. Methods of purification that include a density gradient can include one or more, or two or more, steps of ultracentrifugation.

To provide one non-limiting, exemplary protocol for purification of helper-dependent adenoviral vectors by cesium chloride density gradient purification by ultracentrifugation, viral lysate can be harvested from producer cells and applied to a continuous CsCl step gradient ranging from a density of around 1.4 g/ml at the bottom of the tube to 1.25 g/ml in the top layer. Without wishing to be bound by any particular theory, generalization, or exemplification, certain adenoviral helper-dependent vectors can have a buoyant density of about 1.34 g/ml and can separate from other (e.g. contaminating) agents to collect as a band in about the middle of the gradient after centrifugation. In various embodiments, a collected Ad band can be mixed with 1.35 g/ml CsCl solution and subjected to the second round of isopycnic gradient ultracentrifugation. A helper vector will be separable in the density gradient.

Vector purification can alternatively or additionally (e.g., before or after at least one round of density gradient purification) include purification by chromatography. In various embodiments, the chromatography is ion-exchange chromatography. In various embodiments, the chromatography is affinity chromatography. In various embodiments, the chromatography is gel filtration chromatography. In various embodiments, the chromatography is size-exclusion and/or hydrophobic interaction chromatography. In various embodiments the chromatography includes one or more or two or more steps of chromatography. Chromatography can be used in combination with ultracentrifugation and/or filtration. Vector purification can alternatively or additionally (e.g., after at least one round of density gradient purification) include purification by membrane adsorption.

Vector purification can alternatively or additionally (e.g., before or after at least one round of density gradient purification) include an iodixanol gradient for separating helper from helper dependent vectors. Certain exemplary iodixanol gradients are described, e.g., in Dormond et al. (2010 J. Virol. Meth. 165:83-89). In various embodiments, an exemplary purification process can include (1) anion-exchange chromatography for initial capturing of virus and (2) a shallow iodixanol density gradient ultracentrifugation for purification of helper-dependent virus, and can further include (3) size-exclusion chromatography for the removal of iodixanol and residual protein contaminants.

In various embodiments, a purified helper-dependent genome or helper-dependent vector is characterized in that it is present in a composition in which at least 96% (e.g., at least 96%, 96.5%, 97%, 97.5%, 98%, 98.5%, 99%, 99.1%, 99.2%, 99.3%, 99.4%, 99.5%, 99.6%, 99.7%, 99.8%, 99.9%, 99.95%, 99.99%, 99.995%, 99.999%, or 100%) of adenoviral genomes and/or adenoviral vectors present in the composition are helper-dependent adenoviral genomes and/or helper-dependent adenoviral vectors. In various embodiments, a purified helper-dependent genome or helper-dependent vector is characterized in that it is present in a composition in which at least 99% (e.g., at least 99%, 99.1%, 99.2%, 99.3%, 99.4%, 99.5%, 99.6%, 99.7%, 99.8%, 99.9%, 99.95%, 99.99%, 99.995%, 99.999%, or 100%) of adenoviral genomes and/or adenoviral vectors present in the composition are helper-dependent adenoviral genomes and/or helper-dependent adenoviral vectors. In various embodiments, a purified helper-dependent genome or helper-dependent vector is characterized in that it is present in a composition in which at least 99.9% (e.g., at least 99.9%, 99.95%, 99.99%, 99.995%, 99.999%, or 100%) of adenoviral genomes and/or adenoviral vectors present in the composition are helper-dependent adenoviral genomes and/or helper-dependent adenoviral vectors.

In various embodiments, a purified helper-dependent genome or helper-dependent vector is characterized in that it is present in a composition in which the percentage of adenoviral genomes and/or adenoviral vectors present in the composition that are helper-dependent adenoviral genomes and/or helper-dependent adenoviral vectors is in a range having a lower bound selected from 96%, 96.5%, 97%, 97.5%, 98%, 98.5%, 99%, 99.1%, 99.2%, 99.3%, 99.4%, 99.5%, 99.6%, 99.7%, 99.8%, or 99.9%, and an upper bound selected from 99%, 99.1%, 99.2%, 99.3%, 99.4%, 99.5%, 99.6%, 99.7%, 99.8%, 99.9%, 99.95%, 99.99%, 99.995%, 99.999%, or 100%. In various embodiments, a purified helper-dependent genome or helper-dependent vector is characterized in that it is present in a composition in which the percentage of adenoviral genomes and/or adenoviral vectors present in the composition that are helper-dependent adenoviral genomes and/or helper-dependent adenoviral vectors is between 98% and 100%, between 99% and 100%, between 99.5% and 100%, between 99.9% and 100%, or between 99.99% and 100%.

In various embodiments, a purified helper-dependent genome or helper-dependent vector is characterized in that it is present in a composition in which no more than 2% (e.g., no more than 2%, 1.5%, 1.0%, 0.9%, 0.8%, 0.7%, 0.6%, 0.5%, 0.4%, 0.3%, 0.2%, 0.1%, 0.05%, 0.01%, 0.005%, 0.001%, or 0%) of adenoviral genomes and/or adenoviral vectors present in the composition are helper adenoviral genomes and/or helper adenoviral vectors. In various embodiments, a purified helper-dependent genome or helper-dependent vector is characterized in that it is present in a composition in which no more than 1% (e.g., no more than 1.0%, 0.9%, 0.8%, 0.7%, 0.6%, 0.5%, 0.4%, 0.3%, 0.2%, 0.1%, 0.05%, 0.01%, 0.005%, 0.001%, or 0%) of adenoviral genomes and/or adenoviral vectors present in the composition are helper adenoviral genomes and/or helper adenoviral vectors. In various embodiments, a purified helper-dependent genome or helper-dependent vector is characterized in that it is present in a composition in which no more than 0.1% (e.g., no more than 0.1%, 0.05%, 0.01%, 0.005%, 0.001%, or 0%) of adenoviral genomes and/or adenoviral vectors present in the composition are helper adenoviral genomes and/or helper adenoviral vectors.

In various embodiments, a purified helper-dependent genome or helper-dependent vector is characterized in that it is present in a composition in which the percentage of adenoviral genomes and/or adenoviral vectors present in the composition that are helper adenoviral genomes and/or helper adenoviral vectors is in a range having an upper bound selected from 2%, 1.5%, 1.0%, 0.9%, 0.8%, 0.7%, 0.6%, 0.5%, 0.4%, 0.3%, 0.2%, 0.1%, 0.05%, 0.01%, and a lower bound selected from 0.5%, 0.4%, 0.3%, 0.2%, 0.1%, 0.05%, 0.01%, 0.005%, 0.001%, or 0%. In various embodiments, a purified helper-dependent genome or helper-dependent vector is characterized in that it is present in a composition in which the percentage of adenoviral genomes and/or adenoviral vectors present in the composition that are helper adenoviral genomes and/or helper adenoviral vectors is between 2% and 0%, between 1% and 0%, between 0.5% and 0%, between 0.1% and 0%, or between 0.01% and 0%.

In various embodiments, a composition in which a helper-dependent genome or helper dependent vector of the present disclosure is present is e.g., a liquid solution, e.g., a buffer, combination of excipients, pharmaceutically acceptable carrier, pharmaceutical composition, and/or a dosage form.

### EXAMPLES

The present disclosure includes the identification of positions within an adenoviral packaging sequence at which recombinase sites can be advantageously positioned to produce an adenoviral genome (e.g., a helper genome) conditionally deficient for packaging. Identification of these positions and genomes allows constructions of safer and/or more efficient helper-dependent adenoviral vectors and vector systems. The present Examples provide, among other things, the successful production and use of adenoviral genomes, including adenoviral helper genomes, provided herein, including adenoviral helper genomes that include a conditionally defective packaging sequence and/or an inverted packaging sequences.

### Example 1: Design of Ad35 Helper Genomes

The present Example includes the identification of positions within an Ad35 genome, and particularly within the Ad35 packaging sequence, at which recombinase sites can be positioned for efficient switching between packaging competence and packaging deficiency. Insertion or positioning of the recombinase sites into the packaging sequence will not abrogate Ad35 genome packaging. However, excision of the recombinase-flanked sequences will reduce and/or eliminates packaging of the genome.

The present Example includes alignment of adenoviral packaging sequences to identify putative packaging signals in the Ad35 genome, as shown in FIG. 1A. The present Example further includes selection of particular locations for placement of 5′ (left) and 3′ (right) recombinase sites. Selected left recombinase sites include Ad35 genome positions 224, 171, 195, and 161. Selected right recombinase sites include Ad35 genome positions 402, 479, and 497. The present Example further includes particular pairings of left and right recombinase site positions: position 224 with position 402, position 171 with position 402, position 195 with position 479, and position 161 with position 497 (e.g., in a genome where TAGGGATAACAGGGTAAT (SEQ ID NO: 29) is inserted in place of the canonical sequence corresponding to positions 481-497 in the Ad35 genome to create a recognition site for the restriction enzyme I-SceI). Insertion of a right recombinase site at Ad35 genome position 497 can alternatively be described as being at position 3200 (e.g., in a genome including an E1 deletion, such as a deletion of nucleotide positions 480-3199, 481-3199, or 482-3199).

These positions and pairing are shown in FIG. 1A, and are additionally described in the remainder of this Example.

### Exemplary Construct 1

Construct 1 includes recombinase sites positioned to generate a conditionally defective packaging sequence of an Ad35 helper genome. Inserted LoxP sites are shown in the context of nucleotides 1-402 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). In addition, the sequence CCGGCC (SEQ ID NO: 14) was inserted to create a recognition site for the restriction enzyme FseI (TTATGGCCGGCCGGGTGGAGTTTTTTTGCAAGTTGTCGCGGGAAATGTTACGCATAA AAAGGCTTCTTTTCTCACGGAACTACTTAGTTTTCC) (SEQ ID NO: 15). Further sequence information is provided below.

### Sequence Corresponding to AY128640 Nucleotides 1-402 Engineered to Include a Conditionally Defective Packaging Sequence (LoxP Sequences Underlined)

(SEQ ID NO: 16)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTTTTATGGCCGG
CCGGGTGGAGTTTTTTTGCAAGTTGTCGCGGGAAATGTTACGCATAAAA
AGGCTTCTTTTCTCACGGAACTACTTAGTTTTCCATAACTTCGTATAGC
ATACATTATACGAAGTTATCACGGTATTTAACAGGAAATGAGGTAGTTT
TGACCGGATGCAAGTGAAAATTGCTGATTTTCGCGCGAAAACTGAATGA
GGAAGTGTTTTTCTGAATAATGTGGTATTTATGGCAGGGTGGAGTATTT
GTTCAGGGCCAGGTAGACTTTGACCCATTACGTGGAGGTTTCGATTACC
GATAACTTCGTATAGCATACATTATACGAAGTTAT

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTT

### LoxP Sequence

(SEQ ID NO: 18)
ATAACTTCGTATAGCATACATTATACGAAGTTAT

### LoxP-Flanked Sequence (Identified Packaging Signals A1, A2, A5 and A6 Underlined)

(SEQ ID NO: 19)
CACGGTATTTAACAGGAAATGAGGTAGTTTTGACCGGATGCAAGTGAAAA
TTGCTGATTTTCGCGCGAAAACTGAATGAGGAAGTGTTTTTCTGAATAAT
GTGGTATTTATGGCAGGGTGGAGTATTTGTTCAGGGCCAGGTAGACTTTG
ACCCATTACGTGGAGGTTTCGATTACCG

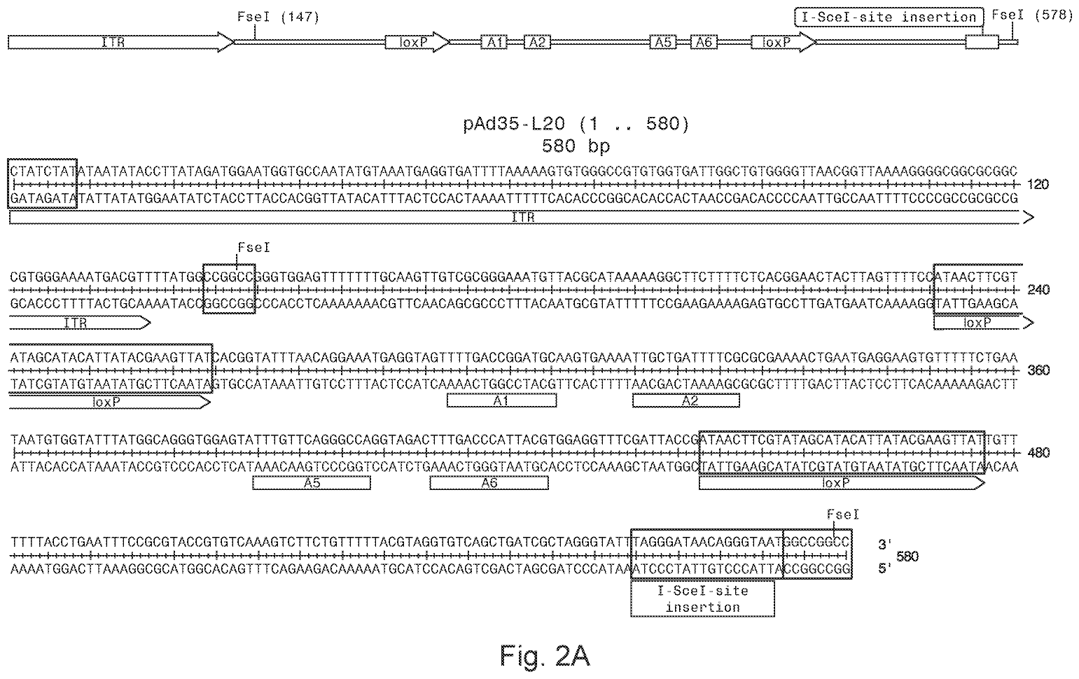

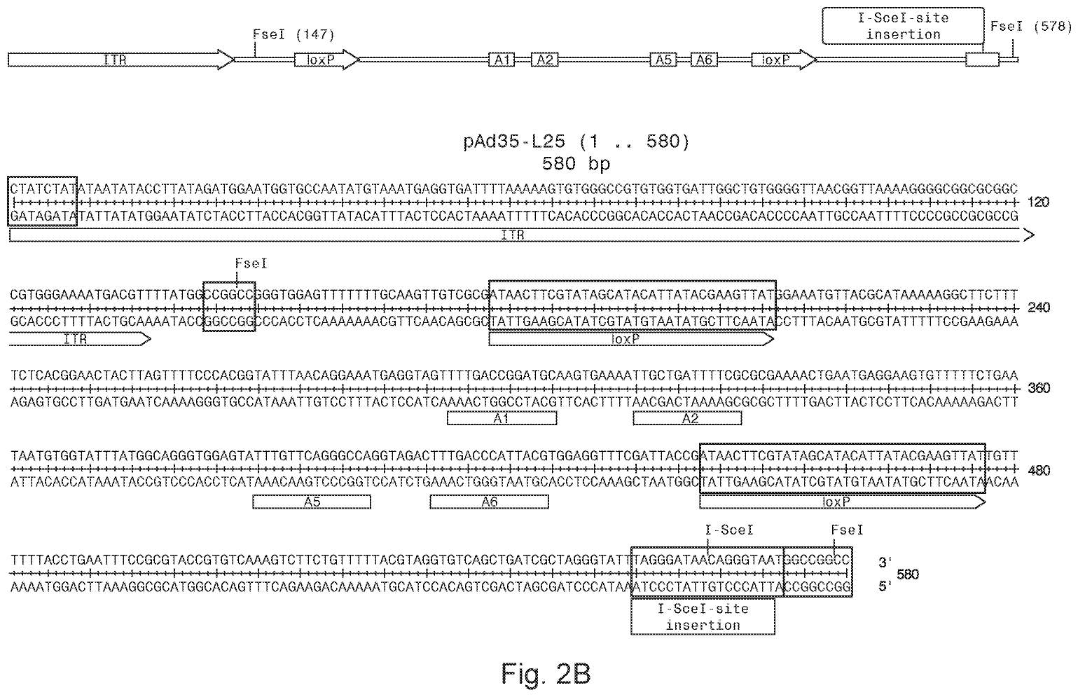

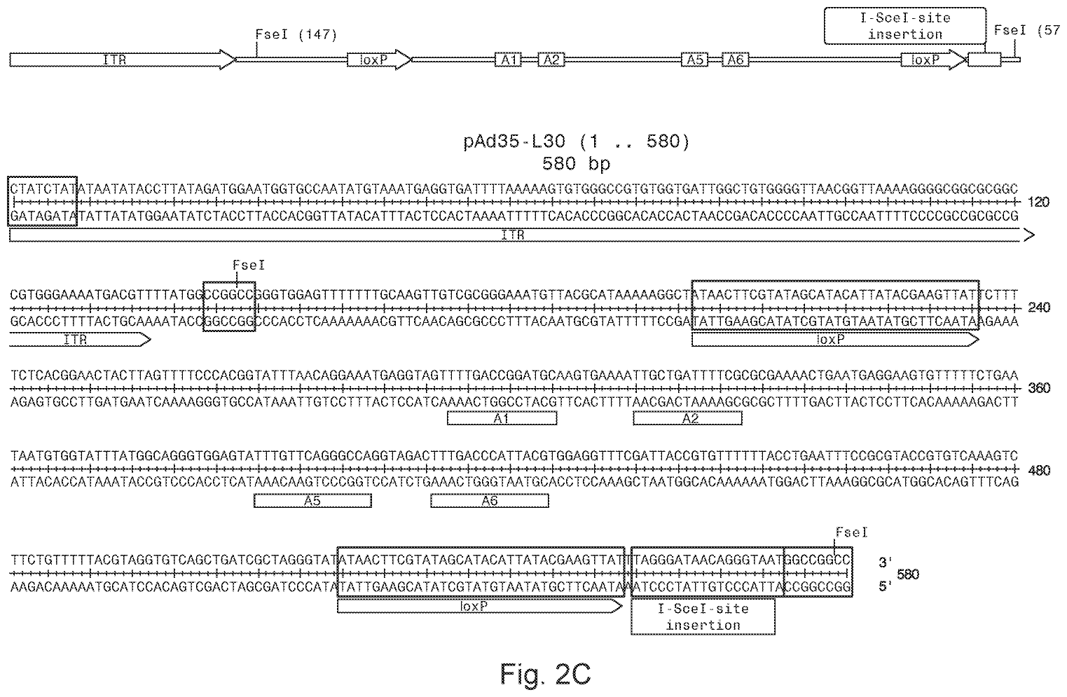

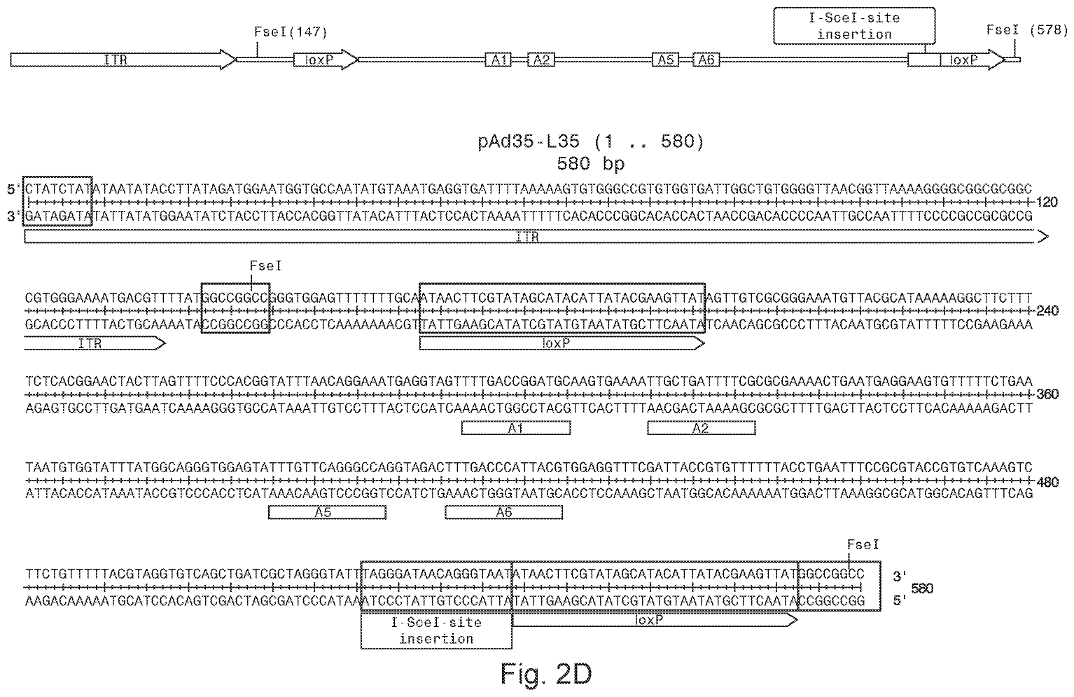

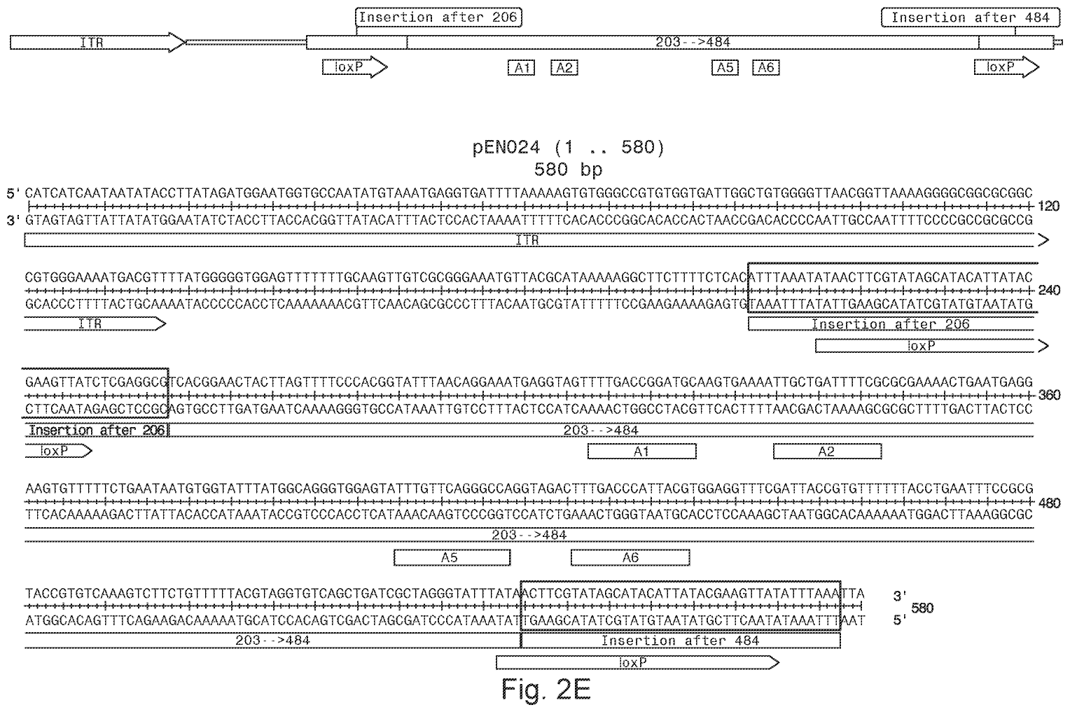

FIG. 2A shows a region (the left end) of an Ad35 helper genome that includes a sequence according to Construct 1.

### Exemplary Construct 2

Construct 2 includes recombinase sites positioned to generate a conditionally defective packaging sequence of an Ad35 helper genome. Inserted LoxP sites are shown in the context of nucleotides 1-402 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. In addition, the sequence CCGGCC (SEQ ID NO: 14) was inserted to create a recognition site for the restriction enzyme FseI (TTATGGCCGGCCGGGTGGAGTTTTTTTGCAAGTIGTCGCG; SEQ ID NO: 20). Further sequence information is provided below.

### Sequence Corresponding to AY128640 Nucleotides 1-402 Engineered to Include a Conditionally Defective Packaging Sequence (LoxP Sequences Underlined)

(SEQ ID NO: 21)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTTTTATGGCCGGCCG
GGTGGAGTTTTTTTGCAAGTTGTCGCGATAACTTCGTATAGCATACATTA
TACGAAGTTATGGAAATGTTACGCATAAAAAGGCTTCTTTTCTCACGGAA
CTACTTAGTTTTCCCACGGTATTTAACAGGAAATGAGGTAGTTTTGACCG
GATGCAAGTGAAAATTGCTGATTTTCGCGCGAAAACTGAATGAGGAAGTG
TTTTTCTGAATAATGTGGTATTTATGGCAGGGTGGAGTATTTGTTCAGGG
CCAGGTAGACTTTGACCCATTACGTGGAGGTTTCGATTACCGATAACTTC
GTATAGCATACATTATACGAAGTTAT

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTT

### LoxP Sequence

(SEQ ID NO: 18)
ATAACTTCGTATAGCATACATTATACGAAGTTAT

### LoxP-Flanked Sequence (Identified Packaging Signals A1, A2, A5 and A6 Underlined)

(SEQ ID NO: 22)
GGAAATGTTACGCATAAAAAGGCTTCTTTTCTCACGGAACTACTTAGTT
TTCCCACGGTATTTAACAGGAAATGAGGTAGTTTTGACCGGATGCAAGT
GAAAATTGCTGATTTTCGCGCGAAAACTGAATGAGGAAGTGTTTTTCTG
AATAATGTGGTATTTATGGCAGGGTGGAGTATTTGTTCAGGGCCAGGTA
GACTTTGACCCATTACGTGGAGGTTTCGATTACCG

FIG. 2B shows a region (the left end) of an Ad35 helper genome that includes a sequence according to Construct 2.

### Exemplary Construct 3

Construct 3 includes recombinase sites positioned to generate a conditionally defective packaging sequence of an Ad35 helper genome. Inserted LoxP sites are shown in the context of nucleotides 1-479 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). In addition, the sequence CCGGCC (SEQ ID NO: 14) was inserted to create a recognition site for the restriction enzyme FseI (TTATGGCCGGCCGGGTGGAGTTTTTTTGCAAGTIGTCGCGGGAAATGTTACGCATAA AAAGGCT; SEQ ID NO: 23). Further sequence information is provided below.

### Sequence Corresponding to AY128640 Nucleotides 1-479 Engineered to Include a Conditionally Defective Packaging Sequence (LoxP Sequences Underlined)

(SEQ ID NO: 24)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTTTTATGGCCGG
CCGGGTGGAGTTTTTTTGCAAGTTGTCGCGGGAAATGTTACGCATAAAA
AGGCTATAACTTCGTATAGCATACATTATACGAAGTTATTCTTTTCTCA
CGGAACTACTTAGTTTTCCCACGGTATTTAACAGGAAATGAGGTAGTTT
TGACCGGATGCAAGTGAAAATTGCTGATTTTCGCGCGAAAACTGAATGA
GGAAGTGTTTTTCTGAATAATGTGGTATTTATGGCAGGGTGGAGTATTT
GTTCAGGGCCAGGTAGACTTTGACCCATTACGTGGAGGTTTCGATTACC
GTGTTTTTTACCTGAATTTCCGCGTACCGTGTCAAAGTCTTCTGTTTTT
ACGTAGGTGTCAGCTGATCGCTAGGGTATATAACTTCGTATAGCATACA
TTATACGAAGTTAT

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTT

### LoxP Sequence

(SEQ ID NO: 18)
ATAACTTCGTATAGCATACATTATACGAAGTTAT

### LoxP-Flanked Sequence (Identified Packaging Signals A1, A2, A5 and A6 Underlined)

(SEQ ID NO: 25)
TCTTTTCTCACGGAACTACTTAGTTTTCCCACGGTATTTAACAGGAAAT
GAGGTAGTTTTGACCGGATGCAAGTGAAAATTGCTGATTTTCGCGCGAA
AACTGAATGAGGAAGTGTTTTTCTGAATAATGTGGTATTTATGGCAGGG
TGGAGTATTTGTTCAGGGCCAGGTAGACTTTGACCCATTACGTGGAGGT
TTCGATTACCGTGTTTTTTACCTGAATTTCCGCGTACCGTGTCAAAGTC
TTCTGTTTTTACGTAGGTGTCAGCTGATCGCTAGGGTAT

FIG. 2C shows a region (the left end) of an Ad35 helper genome that includes a sequence according to Construct 3.

### Exemplary Construct 4

Construct 4 includes recombinase sites positioned to generate a conditionally defective packaging sequence of an Ad35 helper genome. Inserted LoxP sites are shown in the context of nucleotides 1-497 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). In addition, the sequence CCGGCC (SEQ ID NO: 14) was inserted to create a recognition site for the restriction enzyme FseI (TTATGGCCGGCCGGGTGGAGTTTTTTTGCA; SEQ ID NO: 26). Further sequence information is provided below.

### Sequence Corresponding to AY128640 Nucleotides 1-480 Engineered to Include a Conditionally Defective Packaging Sequence (LoxP Sequences Underlined)

(SEQ ID NO: 27)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTTTTATGGCCGG
CCGGGTGGAGTTTTTTTGCAATAACTTCGTATAGCATACATTATACGAA
GTTATAGTTGTCGCGGGAAATGTTACGCATAAAAAGGCTTCTTTTCTCA
CGGAACTACTTAGTTTTCCCACGGTATTTAACAGGAAATGAGGTAGTTT
TGACCGGATGCAAGTGAAAATTGCTGATTTTCGCGCGAAAACTGAATGA
GGAAGTGTTTTTCTGAATAATGTGGTATTTATGGCAGGGTGGAGTATTT
GTTCAGGGCCAGGTAGACTTTGACCCATTACGTGGAGGTTTCGATTACC
GTGTTTTTTACCTGAATTTCCGCGTACCGTGTCAAAGTCTTCTGTTTTT
ACGTAGGTGTCAGCTGATCGCTAGGGTATTTAGGGATAACAGGGTAATA
TAACTTCGTATAGCATACATTATACGAAGTTAT

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTT

### LoxP Sequence

(SEQ ID NO: 18)
ATAACTTCGTATAGCATACATTATACGAAGTTAT

### LoxP-Flanked Sequence (Identified Packaging Signals A1, A2, A5 and A6 Underlined; Sequence Inserted in Place of a Deletion of Base Pairs 481-497 Bold and Italicized)

(SEQ ID NO: 28)
AGTTGTCGCGGGAAATGTTACGCATAAAAAGGCTTCTTTTCTCACGGAAC
TACTTAGTTTTCCCACGGTATTTAACAGGAAATGAGGTAGTTTTGACCGG
ATGCAAGTGAAAATTGCTGATTTTCGCGCGAAAACTGAATGAGGAAGTGT
TTTTCTGAATAATGTGGTATTTATGGCAGGGTGGAGTATTTGTTCAGGGC
CAGGTAGACTTTGACCCATTACGTGGAGGTTTCGATTACCGTGTTTTTTA
CCTGAATTTCCGCGTACCGTGTCAAAGTCTTCTGTTTTTACGTAGGTGTC
AGCTGATCGCTAGGGTATTTAGGGATAACAGGGTAAT

FIG. 2D shows a region (the left end) of an Ad35 helper genome that includes a sequence according to Construct 4.

### Example 2: Analysis of Ad35 Helper Genome Propagation and Stability

The present Example demonstrates that Ad35 helper genomes including recombinase-flanked packaging sequences according to the present disclosure are stable and can be propagated without detectable genome rearrangement.

A helper genome can be present in a plasmid or in a viral vector. Plasmid forms can be used to transfect target cells for production of helper vectors (which helper vectors include the Ad35 helper genome) or for production of donor vectors (which donor vectors do not include the Ad35 helper genome). Four plasmids encoding E1-deleted Ad35 helper genomes (designated pEN025, pEN026, pEN027, and pEN028), were each transfected into HEK293 cells and propagated to determine whether viable helper viruses could be rescued. Each of pEN025, pEN026, pEN027, and pEN028 included a construct according to Constructs 1˜4 in Example 1, respectively.

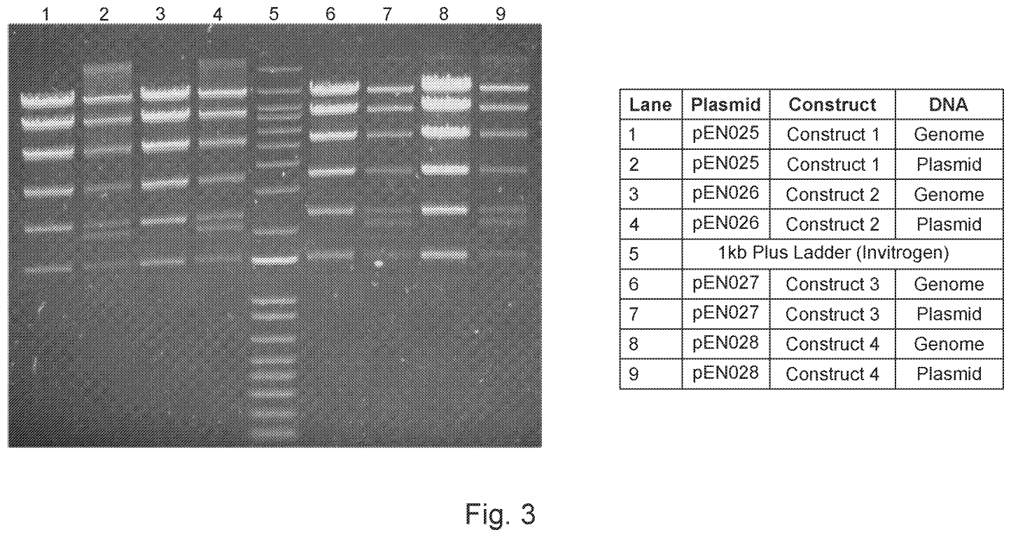

Rescued E1-deleted adenoviruses were purified using standard methods (see, e.g., Su et al. doi:10.1101/pdb.prot095547 Cold Spring Harb Protoc 2019) and viral genomes were isolated from purified helper vectors. Isolated Ad35 helper genomes were digested with BsrGI alone, and starting plasmids were digested with BsrGI and SwaI (which excises the plasmid backbone sequence) for comparison. Digestion products were analyzed by gel electrophoresis (FIG. 3).

To determine whether the Ad35 helper genomes were stable during propagation the restriction patterns obtained by digesting isolated adenoviral genomic DNA were compared to the restriction patterns obtained by digesting starting plasmids with the restriction enzymes BsrG1 and SwaI. Analysis of the restriction patterns on a gel showed the expected banding pattern and expected band sizes (FIG. 3), demonstrating that that Ad35 helper genomes including recombinase-flanked packaging sequences as disclosed herein are genetically stable and can be propagated without detectable genome rearrangement in large-scale preparations.

### Example 3: Analysis of Recombinase-Mediated Excision of Recombinase-Flanked Packaging Sequences in Ad35 Helper Genomes

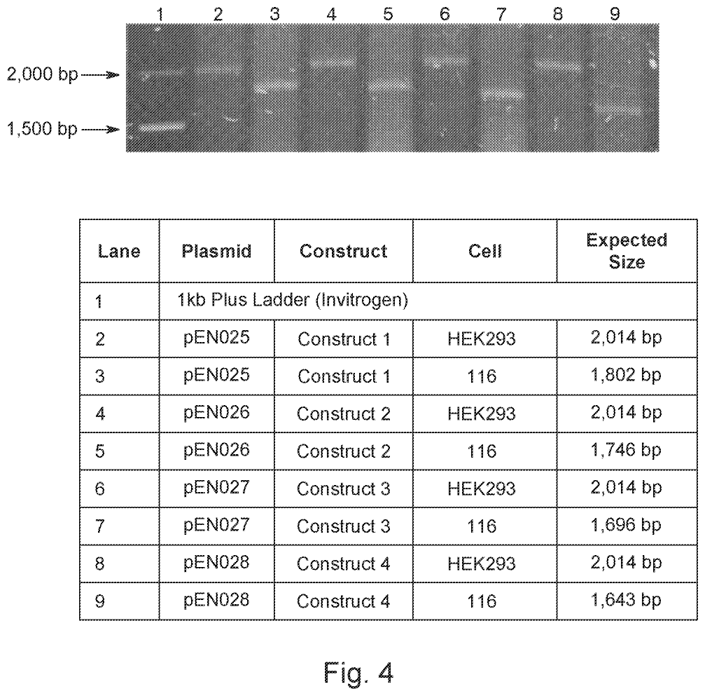

The present Example demonstrates the recombinase-mediated deletion of recombinase-flanked packaging sequences in Ad35 helper genomes. Plasmids including Ad35 helper genomes (pEN025, pEN026, pEN027, and pEN028) were linearized by digestion with SwaI (which excised the plasmid backbone sequence) and transfected into each of two cell types: HEK293 cells that do not express Cre recombinase, and 116 cells modified from HEK293 cells to express Cre recombinase. Thus, excision of loxP flanked sequences is expected in the 116 cells but not the HEK293 cells. DNA was isolated from transfected cells and digested with the restriction enzyme ApaI. Digestion of the Ad35 helper genome with restriction enzyme ApaI is expected to produce a 2014 bp fragment. A smaller DNA fragment is expected if the Ad35 helper genome has undergone recombination to mediate deletion of the recombinase-flanked packaging sequence. Restriction results were analyzed by gel electrophoresis (FIG. 4). The expected band sizes were observed for DNA isolated from HEK293 cells transfected with the Ad35 helper genomes (FIG. 4—lanes 2, 4, 6, and 8) and for DNA isolated from 116 cells transfected with the Ad35 helper genomes (FIG. 4—lanes 3, 5, 7, and 9). Data therefore show successful Cre-mediated excision of flanked packaging sequences from all helper genomes in the presence of recombinase.

### Example 4: Analysis of Helper-Dependent Adenovirus (HDAd) Production Using Ad35 Helper Vectors with Genomes Including Recombinase-Flanked Packaging Sequences

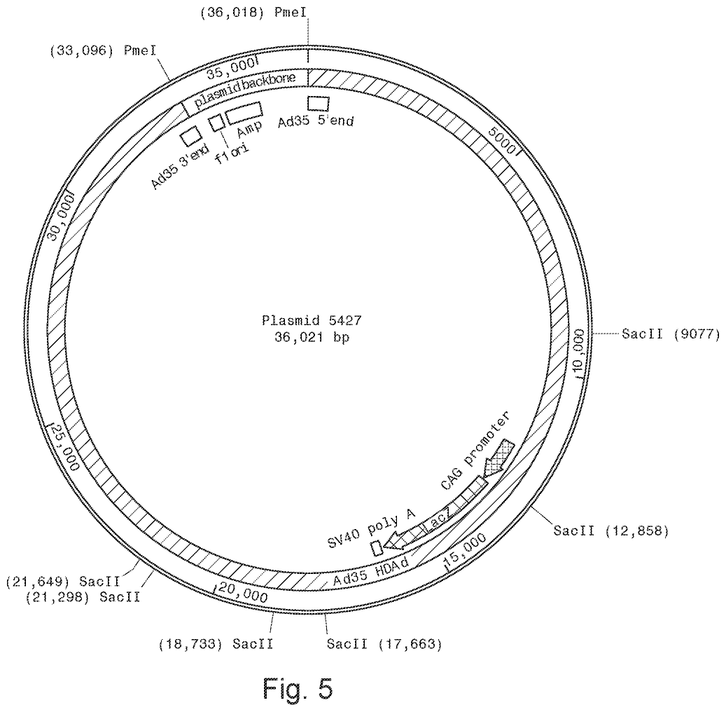

The present Example demonstrates the production of helper-dependent adenovirus (HDAd) using Ad35 helper vectors with genomes including recombinase-flanked packaging sequences. Ad35 helper vectors were purified from HEK293 cells transfected with plasmids including Ad35 helper genomes with recombinase-flanked packaging sequences (pEN025, pEN026, pEN027, and pEN028, and pEN024). Helper-dependent adenoviral vectors were then produced according to standard procedures (see Palmer and Ng, Methods Mol Biol. 2008; 433:33-53) in 116 cells using the purified Ad35 helper vectors and transfecting plasmid 5427, a plasmid that encodes a helper-dependent genome that includes terminal sequences derived from Ad35 and includes a cassette for expression of beta-galactosidase (FIG. 5). HDAd viral particles produced using Ad35 helper vectors from pEN026 and pEN028 were isolated and subsequently used to achieve production of secondary HDAd preparations by co-infection of 116 cells with the HDAd viral particles from plasmid 5427 and Ad35 helper viral particles from pEN026 or pEN028 (respectively).

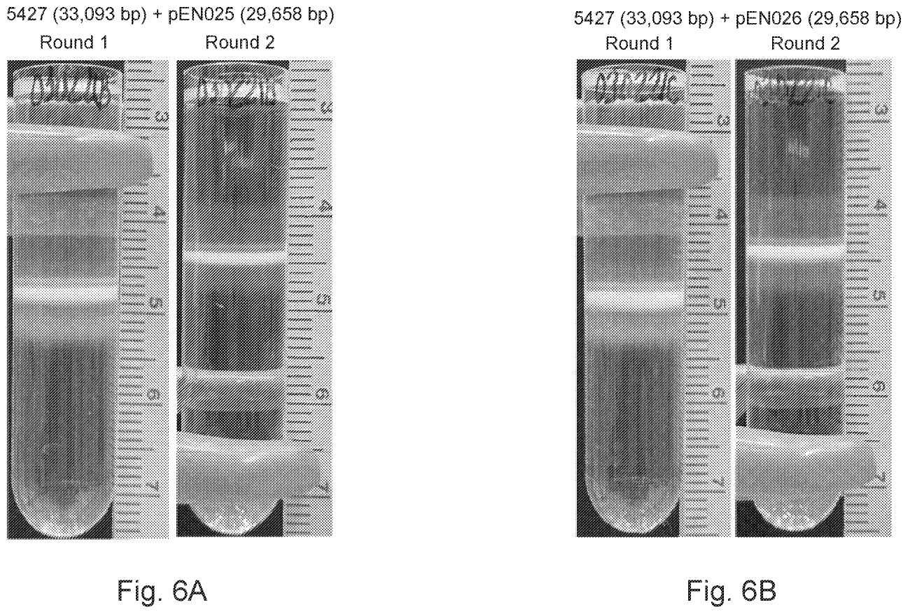

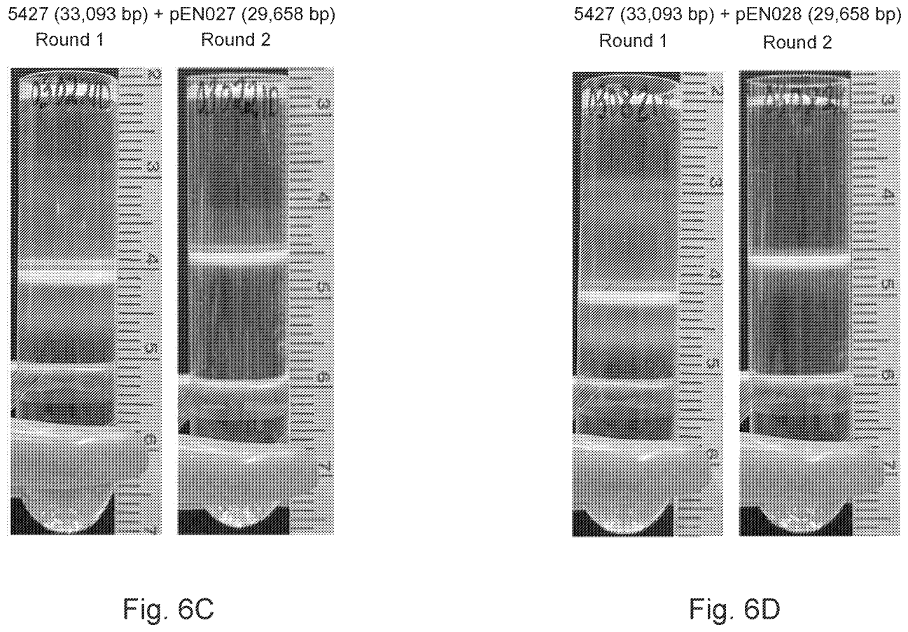

Helper-dependent adenovirus (HDAd) preparations were purified by using two consecutive cesium chloride continuous gradients (FIG. 6A-E). Purified HDAd preparations were characterized using several approaches. The physical titer or yield of the purified virus preparations was determined by spectrophotometry and can be expressed as the total number of purified viral particles (vp) or the number of viral particles per volume (vp/ml). The infectivity of the purified HDAd preparations was determined by using the purified helper-dependent viruses to infect cultured HEK293 cells and staining the cells to determine their expression of beta-galactosidase (as described in Parks et al., PNAS. 1996: 93(24):13565-13570). Infected cells were expected to express beta-galactosidase. Infectivity was represented in terms of blue-forming units (BFU), which is the number of cells that showing blue staining indicating positive expression of beta-galactosidase encoded by the cassette in HDAd genome. Infectivity can be further represented as the BFU per volume of purified virus (BFU/ml) and/or the ratio between the total number of viral particles and the BFU (vp:BFU).

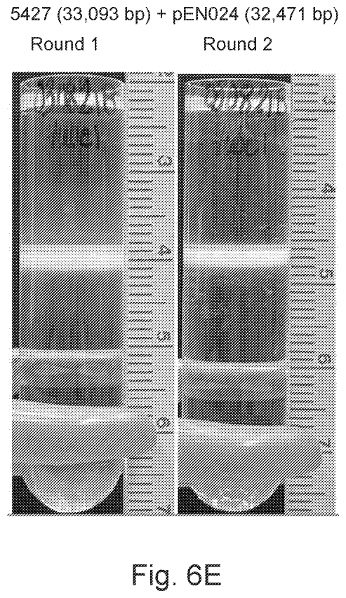

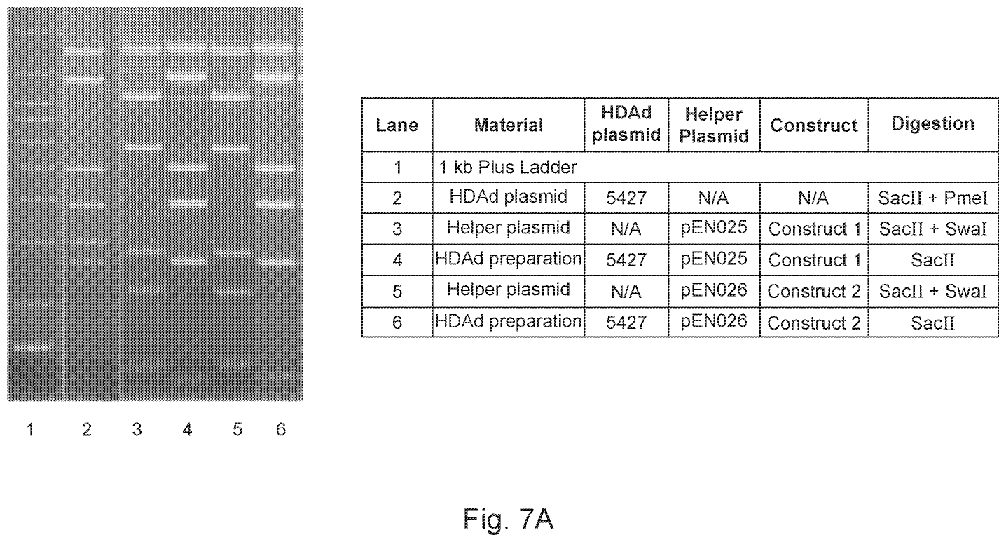

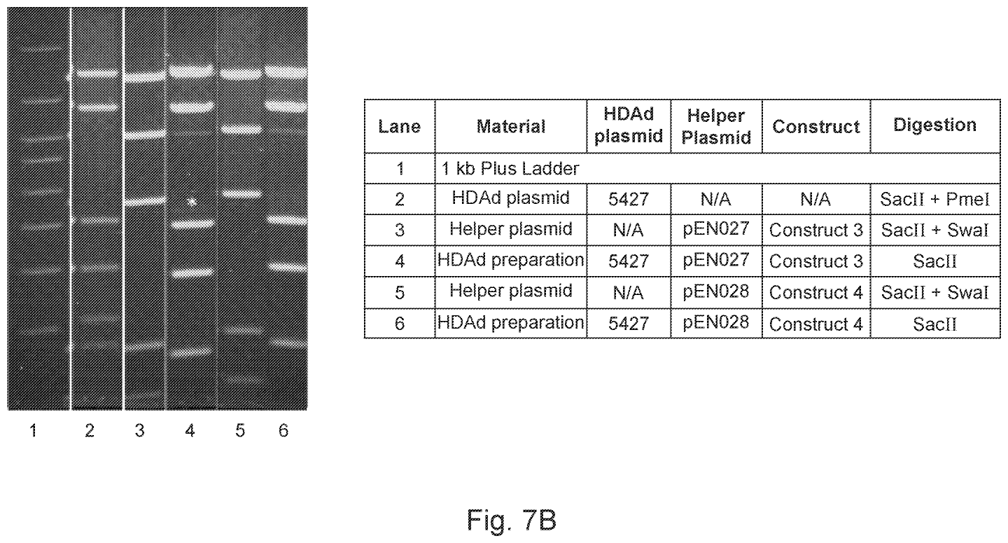

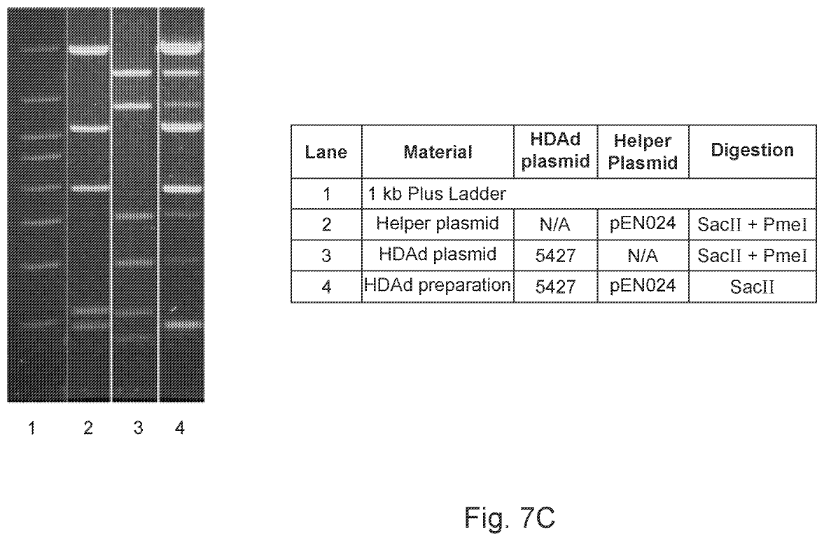

Further characterization of the purified HDAd preparations was performed using DNA isolated from the purified HDAd preparations. Isolated DNA was digested using restriction enzyme (SacII) and the restriction pattern was compared to the restriction pattern obtained by digestion using restriction enzymes (SacII and PmeI) of the starting HDAd plasmid and the restriction pattern obtained by digestion using restriction enzymes (SacII and SwaI for pEN025, pEN026, pEN027, and pEN028; or SacII and PmeI for pEN024) of the starting Ad35 helper plasmids. Analysis of the restriction patterns on a gel showed the expected banding pattern and expected band sizes (FIG. 7A-C), indicating successful HDAd production. While in FIGS. 7A and 7B the restriction patterns of the HDAd preparations demonstrate a low level of helper virus contamination, in FIG. 7C, the banding pattern in lane 4 demonstrates comparatively greater helper virus contamination. The HDAd preparation examined in FIG. 7C was prepared using Ad35 helper vectors produced using a plasmid (pEN024) encoding an Ad35 helper vector genome that includes the construct of FIG. 2E. Notwithstanding, vectors, genomes, and conditional packaging sequences analyzed in FIGS. 7A-C are advantageous and useful for various methods and compositions provided herein. Additionally, the Ad35 helper contamination fraction in the purified preparation was determined using quantitative PCR of DNA isolated from the purified HDAd preparation.

Table 36 shows the results from experiments to characterize the purified HDAd preparations. Table 37 shows results from secondary preparations, including estimated helper fraction (%).

TABLE 36
Characterization of Purified HDAd Preparations

| Helper | | Yield | Yield | Infectivity | Infectivity | Helper fraction |
|---|---|---|---|---|---|---|
| plasmid | Construct | (vp) | (vp/ml) | (BFU/ml) | (vp:BFU) | (%) |
| pEN025 | Construct 1 | 2.54e12 | 7.9e11 | 5.39e10 | 14.7:1 | 8.99 |
| pEN026 | Construct 2 | 3.59e12 | 1.25e12 | 1.05e11 | 11.9:1 | 8.10 |
| pEN027# | Construct 3 | 2.73e12 | 1.06e12 | 4.59e10 | 21.4:1 | 5.93 |
| pEN028 | Construct 4 | 2.43e12 | 9.51e11 | 7.22e11 | 13.2:1 | 7.05 |
| pEN024 | | 1.32e13 | 2.28e12 | 2.29e10 | 99.6:1 | 89.2 |

“#” indicates that evidence of genome rearrangement was observed in HDAd preparations generated using this helper plasmid (indicated by band below asterisk in FIG. 7B).

Notwithstanding, vectors, genomes, and conditional packaging sequences associated with such helper plasmids can be advantageous and useful for certain methods and compositions provided herein.

TABLE 37
Characterization of Secondary HDAd Preparations

| | | | | Helper |
|---|---|---|---|---|
| Helper | | Yield | Infectivity | fraction |
| plasmid | Construct | (vp) | (vp:BFU) | (%) |
| pEN026 | Construct 2 | 6.66e12 | 8.35:1 | 2.9 |
| pEN028 | Construct 4 | 3.82e12 | 7.86:1 | 3.1 |

### Example 5: Design of Ad35 Helper Genomes Including Inverted Packaging Sequences

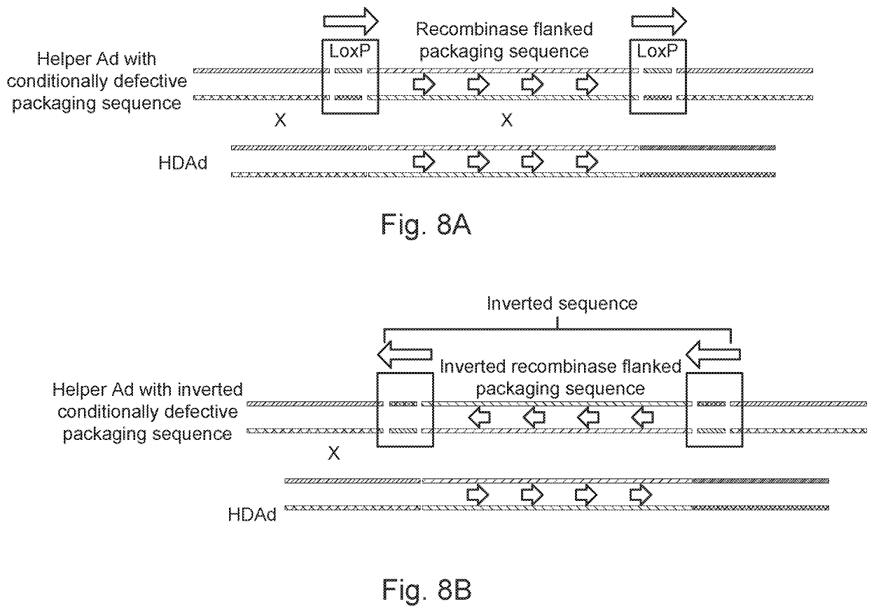

The present Example demonstrates the design of Ad35 helper genomes that include inverted packaging sequences. The present Example is based at least in part on the recognition that use of an inverted packaging sequence in an Ad35 helper vector can reduce and/or eliminate recombinase site-excising homologous recombination (compare FIG. 8A and FIG. 8B). Inversion of sequences comprising a conditionally defective packaging sequence—thereby generating an inverted recombinase-flanked packaging sequences—will reduce and/or eliminate recombinase site-excising homologous recombination, as shown in FIG. 8B. Sequence elements included within an inverted sequence are herein referred to as inverted sequence elements (e.g., a recombinase-flanked packaging sequence included within an inverted sequence is referred to as an inverted recombinase-flanked packaging sequence). A person of skill in the art would appreciate from the present disclosure that the orientation of the packaging sequence is not critical to its function (see, e.g., Palmer and Ng, Mol Ther. 2003; 8:8460852) and would further appreciate from the present disclosure that an inverted conditionally defective packaging sequence as disclosed herein is packaging competent. An inverted recombinase site flanked packaging sequence can further be excised by recombination upon contact with a corresponding recombinase and prevent packaging of a helper genome.

The present Example particularly includes Ad35 helper genomes including inverted packaging sequences as set forth below.

### Exemplary Construct 5

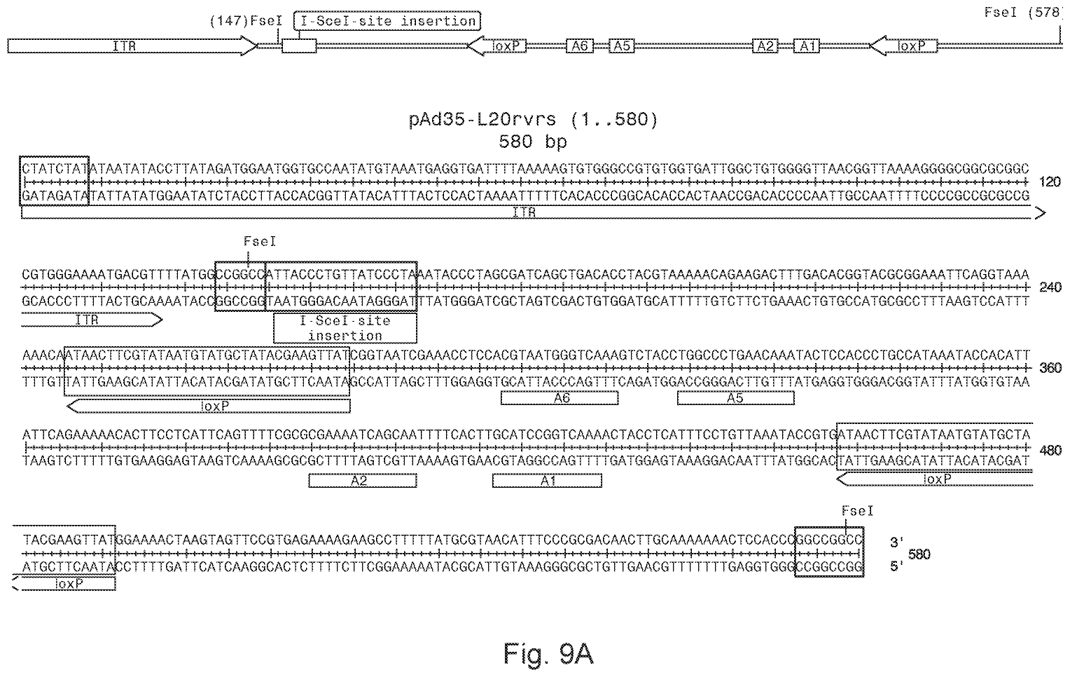

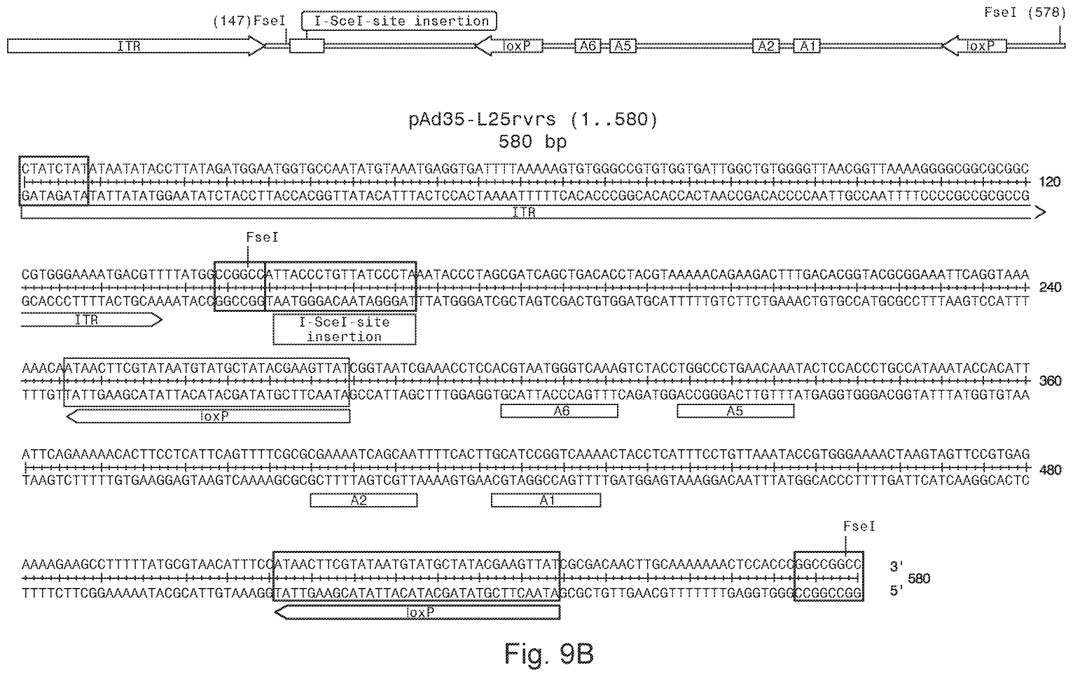

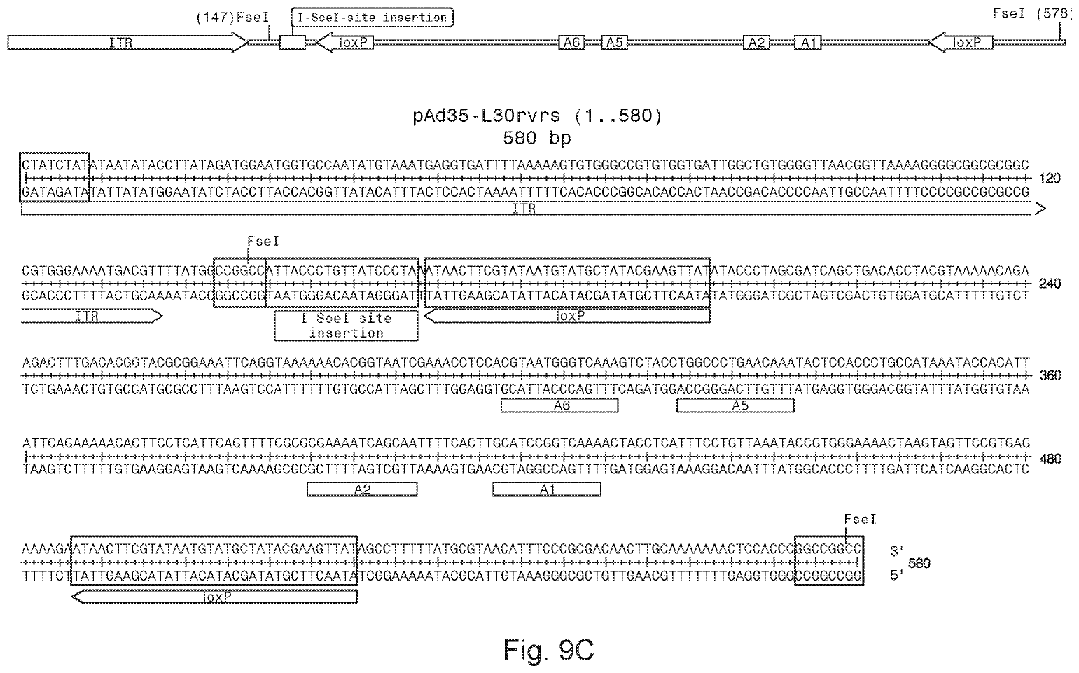

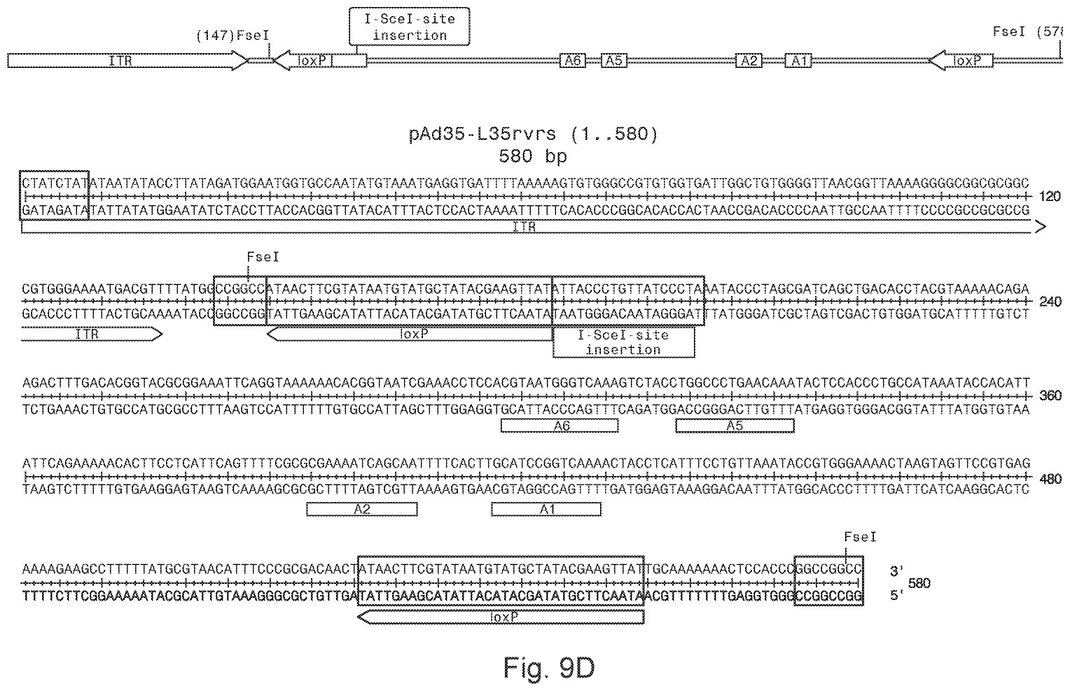

Construct 5 (FIG. 9A) corresponds to Construct 1 (FIG. 2A) but includes a packaging sequence inversion. The inverted recombinase-flanked packing sequence is shown in the context of nucleotides 1-497 of the reference Ad35 sequence in GenBank accession number AY128640. Positions of inserted sequence elements are identified based on their correspondence with positions of the reference Ad35 genome sequence, including if present in Construct 5 in an inverted orientation. Two inserted LoxP sites—one at position 224 and the other at position 402—flank a packaging sequence of the Ad35 genome, so that Cre recombinase-mediated deletion of the flanked packaging sequence will render the genome deficient for packaging. The sequence TAGGGATAACAGGGTAAT (SEQ ID NO: 29) was inserted in place of a deletion of base pairs 481-497 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 30) was inserted at position 497 to create a second recognition site for the restriction enzyme FseI. An inverted recombinase-flanked packing sequence was generated by inversion of a sequence comprising the recombinase-flanked packaging sequence. The inverted sequence for Construct 5 includes the sequence flanked by the two FseI sites at positions 143 and 497-which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). FIG. 9A shows a region of an Ad35 helper genome that includes Construct 5. Sequence information is provided below.

### Sequence Corresponding to AY128640 Nucleotides 1-497 Engineered to Include an Inverted Recombinase-Flanked Packing Sequence (Inverted Sequence Underlined; FseI Recognition Sites Bold and Italicized)

(SEQ ID NO: 31)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTTTTATGGCCGG
CCATTACCCTGTTATCCCTAAATACCCTAGCGATCAGCTGACACCTACG
TAAAAACAGAAGACTTTGACACGGTACGCGGAAATTCAGGTAAAAAACA
ATAACTTCGTATAATGTATGCTATACGAAGTTATCGGTAATCGAAACCT
CCACGTAATGGGTCAAAGTCTACCTGGCCCTGAACAAATACTCCACCCT
GCCATAAATACCACATTATTCAGAAAAACACTTCCTCATTCAGTTTTCG
CGCGAAAATCAGCAATTTTCACTTGCATCCGGTCAAAACTACCTCATTT
CCTGTTAAATACCGTGATAACTTCGTATAATGTATGCTATACGAAGTTA
TGGAAAACTAAGTAGTTCCGTGAGAAAAGAAGCCTTTTTATGCGTAACA
TTTCCCGCGACAACTTGCAAAAAAACTCCACCCGGCCGGCC

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTT

### Inverted LoxP Sequence

(SEQ ID NO: 1)
ATAACTTCGTATAATGTATGCTATACGAAGTTAT

### Inverted Sequence (Inverted LoxP Sequences Underlined; Inverted Recombinase-Flanked Packaging Sequence Bold and Italicized)

(SEQ ID NO: 32)
ATTACCCTGTTATCCCTAAATACCCTAGCGATCAGCTGACACCTACGTA
AAAACAGAAGACTTTGACACGGTACGCGGAAATTCAGGTAAAAAACAAT
AACTTCGTATAATGTATGCTATACGAAGTTATCGGTAATCGAAACCTCC
ACGTAATGGGTCAAAGTCTACCTGGCCCTGAACAAATACTCCACCCTGC
CATAAATACCACATTATTCAGAAAAACACTTCCTCATTCAGTTTTCGCG
CGAAAATCAGCAATTTTCACTTGCATCCGGTCAAAACTACCTCATTTCC
TGTTAAATACCGTGATAACTTCGTATAATGTATGCTATACGAAGTTATG
GAAAACTAAGTAGTTCCGTGAGAAAAGAAGCCTTTTTATGCGTAACATT
TCCCGCGACAACTTGCAAAAAAACTCCACCC

### Inverted Recombinase-Flanked Packaging Sequence (Identified Inverted Packaging Signals A1, A2, A5 and A6 Underlined)

(SEQ ID NO: 33)
CGGTAATCGAAACCTCCACGTAATGGGTCAAAGTCTACCTGGCCCTGAA
CAAATACTCCACCCTGCCATAAATACCACATTATTCAGAAAAACACTTC
CTCATTCAGTTTTCGCGCGAAAATCAGCAATTTTCACTTGCATCCGGTC
AAAACTACCTCATTTCCTGTTAAATACCGTG

### Exemplary Construct 6

Construct 6 (FIG. 9B) corresponds to Construct 1 (FIG. 2B) but includes a packaging sequence inversion. The inverted recombinase-flanked packing sequence is shown in the context of nucleotides 1-497 of the reference Ad35 sequence in GenBank accession number AY128640. Positions of inserted sequence elements are identified based on their correspondence with positions of the reference Ad35 genome sequence, including if present in Construct 6 in an inverted orientation. Two inserted LoxP sites—one at position 171 and the other at position 402—flank a packaging sequence of the Ad35 genome, so that Cre recombinase-mediated deletion of the flanked packaging sequence will render the genome deficient for packaging. The sequence TAGGGATAACAGGGTAAT (SEQ ID NO: 29) was inserted in place of a deletion of base pairs 481-497 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 30) was inserted at position 497 to create a second recognition site for the restriction enzyme FseI. An inverted recombinase-flanked packing sequence was generated by inversion of a sequence comprising the recombinase-flanked packaging sequence. The inverted sequence for Construct 6 includes the sequence flanked by the two FseI sites at positions 143 and 497—which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). FIG. 9B shows a region of an Ad35 helper genome that includes Construct 6. Sequence information is provided below.

### Sequence Corresponding to AY128640 Nucleotides 1-497 Engineered to Include an Inverted Recombinase-Flanked Packing Sequence (Inverted Sequence Underlined; FseI Recognition Sites Bold and Italicized)

(SEQ ID NO: 34)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTTTTATGGCCGGCCA
TTACCCTGTTATCCCTAAATACCCTAGCGATCAGCTGACACCTACGTAAA
AACAGAAGACTTTGACACGGTACGCGGAAATTCAGGTAAAAAACAATAAC
TTCGTATAATGTATGCTATACGAAGTTATCGGTAATCGAAACCTCCACGT
AATGGGTCAAAGTCTACCTGGCCCTGAACAAATACTCCACCCTGCCATAA
ATACCACATTATTCAGAAAAACACTTCCTCATTCAGTTTTCGCGCGAAAA
TCAGCAATTTTCACTTGCATCCGGTCAAAACTACCTCATTTCCTGTTAAA
TACCGTGGGAAAACTAAGTAGTTCCGTGAGAAAAGAAGCCTTTTTATGCG
TAACATTTCCATAACTTCGTATAATGTATGCTATACGAAGTTATCGCGAC
AACTTGCAAAAAAACTCCACCCGGCCGGCC

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTT

### Inverted LoxP Sequence

(SEQ ID NO: 1)
ATAACTTCGTATAATGTATGCTATACGAAGTTAT

### Inverted Sequence (Inverted LoxP Sequences Underlined; Inverted Recombinase-Flanked Packaging Sequence Bold and Italicized)

(SEQ ID NO: 35)
ATTACCCTGTTATCCCTAAATACCCTAGCGATCAGCTGACACCTACGTAA
AAACAGAAGACTTTGACACGGTACGCGGAAATTCAGGTAAAAAACAATAA
CTTCGTATAATGTATGCTATACGAAGTTATCGGTAATCGAAACCTCCACG
TAATGGGTCAAAGTCTACCTGGCCCTGAACAAATACTCCACCCTGCCATA
AATACCACATTATTCAGAAAAACACTTCCTCATTCAGTTTTCGCGCGAAA
ATCAGCAATTTTCACTTGCATCCGGTCAAAACTACCTCATTTCCTGTTAA
ATACCGTGGGAAAACTAAGTAGTTCCGTGAGAAAAGAAGCCTTTTTATGC
GTAACATTTCCATAACTTCGTATAATGTATGCTATACGAAGTTATCGCGA
CAACTTGCAAAAAAACTCCACCC

### Inverted Recombinase-Flanked Packaging Sequence (Identified Inverted Packaging Signals A1, A2, A5 and A6 Underlined)

(SEQ ID NO: 36)
CGGTAATCGAAACCTCCACGTAATGGGTCAAAGTCTACCTGGCCCTGAAC
AAATACTCCACCCTGCCATAAATACCACATTATTCAGAAAAACACTTCCT
CATTCAGTTTTCGCGCGAAAATCAGCAATTTTCACTTGCATCCGGTCAAA
ACTACCTCATTTCCTGTTAAATACCGTGGGAAAACTAAGTAGTTCCGTGA
GAAAAGAAGCCTTTTTATGCGTAACATTTCC

### Exemplary Construct 7

Construct 7 (FIG. 9C) corresponds to Construct 1 (FIG. 2C) but includes a packaging sequence inversion. The inverted recombinase-flanked packing sequence is shown in the context of nucleotides 1-497 of the reference Ad35 sequence in GenBank accession number AY128640. Positions of inserted sequence elements are identified based on their correspondence with positions of the reference Ad35 genome sequence, including if present in Construct 7 in an inverted orientation. Two inserted LoxP sites—one at position 195 and the other at position 479—flank a packaging sequence of the Ad35 genome, so that Cre recombinase-mediated deletion of the flanked packaging sequence will render the genome deficient for packaging. The sequence TAGGGATAACAGGGTAAT (SEQ ID NO: 29) was inserted in place of a deletion of base pairs 481-497 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 30) was inserted at position 497 to create a second recognition site for the restriction enzyme FseI. An inverted recombinase-flanked packing sequence was generated by inversion of a sequence comprising the recombinase-flanked packaging sequence. The inverted sequence for Construct 7 includes the sequence flanked by the two FseI sites at positions 143 and 497-which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). FIG. 9C shows a region of an Ad35 helper genome that includes Construct 7. Sequence information is provided below.

### Sequence Corresponding to AY128640 Nucleotides 1-497 Engineered to Include an Inverted Recombinase-Flanked Packing Sequence (Inverted Sequence Underlined; FseI Recognition Sites Bold and Italicized)

(SEQ ID NO: 37)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTTTTATGGCCGGCCA
TTACCCTGTTATCCCTAAATAACTTCGTATAATGTATGCTATACGAAGTT
ATATACCCTAGCGATCAGCTGACACCTACGTAAAAACAGAAGACTTTGAC
ACGGTACGCGGAAATTCAGGTAAAAAACACGGTAATCGAAACCTCCACGT
AATGGGTCAAAGTCTACCTGGCCCTGAACAAATACTCCACCCTGCCATAA
ATACCACATTATTCAGAAAAACACTTCCTCATTCAGTTTTCGCGCGAAAA
TCAGCAATTTTCACTTGCATCCGGTCAAAACTACCTCATTTCCTGTTAAA
TACCGTGGGAAAACTAAGTAGTTCCGTGAGAAAAGAATAACTTCGTATAA
TGTATGCTATACGAAGTTATAGCCTTTTTATGCGTAACATTTCCCGCGAC
AACTTGCAAAAAAACTCCACCCGGCCGGCC

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATGA
GGTGATTTTAAAAAGTGTGGGCCGTGTGGTGATTGGCTGTGGGGTTAACG
GTTAAAAGGGGCGGCGCGGCCGTGGGAAAATGACGTT

### Inverted LoxP Sequence

(SEQ ID NO: 1)
ATAACTTCGTATAATGTATGCTATACGAAGTTAT

### Inverted Sequence (Inverted LoxP Sequences Underlined; Inverted Recombinase-Flanked Packaging Sequence Bold and Italicized)

(SEQ ID NO: 38)
ATTACCCTGTTATCCCTAAATAACTTCGTATAATGTATGCTATACGAAG
TTATATACCCTAGCGATCAGCTGACACCTACGTAAAAACAGAAGACTTT
GACACGGTACGCGGAAATTCAGGTAAAAAACACGGTAATCGAAACCTCC
ACGTAATGGGTCAAAGTCTACCTGGCCCTGAACAAATACTCCACCCTGC
CATAAATACCACATTATTCAGAAAAACACTTCCTCATTCAGTTTTCGCG
CGAAAATCAGCAATTTTCACTTGCATCCGGTCAAAACTACCTCATTTCC
TGTTAAATACCGTGGGAAAACTAAGTAGTTCCGTGAGAAAAGAATAACT
TCGTATAATGTATGCTATACGAAGTTATAGCCTTTTTATGCGTAACATT
TCCCGCGACAACTTGCAAAAAAACTCCACCC

### Inverted Recombinase-Flanked Packaging Sequence (Identified Inverted Packaging Signals A1, A2, A5 and A6 Underlined)

(SEQ ID NO: 39)
ATACCCTAGCGATCAGCTGACACCTACGTAAAAACAGAAGACTTTGACA
CGGTACGCGGAAATTCAGGTAAAAAACACGGTAATCGAAACCTCCACGT
AATGGGTCAAAGTCTACCTGGCCCTGAACAAATACTCCACCCTGCCATA
AATACCACATTATTCAGAAAAACACTTCCTCATTCAGTTTTCGCGCGAA
AATCAGCAATTTTCACTTGCATCCGGTCAAAACTACCTCATTTCCTGTT
AAATACCGTGGGAAAACTAAGTAGTTCCGTGAGAAAAGA

### Exemplary Construct 8

Construct 8 (FIG. 9D) corresponds to Construct 1 (FIG. 2D) but includes a packaging sequence inversion. The inverted recombinase-flanked packing sequence is shown in the context of nucleotides 1-497 of the reference Ad35 sequence in GenBank accession number AY128640. Positions of inserted sequence elements are identified based on their correspondence with positions of the reference Ad35 genome sequence, including if present in Construct 8 in an inverted orientation. Two inserted LoxP sites—one at position 161 and the other at position 497—flank a packaging sequence of the Ad35 genome, so that Cre recombinase-mediated deletion of the flanked packaging sequence will render the genome deficient for packaging. The sequence TAGGGATAACAGGGTAAT (SEQ ID NO: 29) was inserted in place of a deletion of base pairs 481-497 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 30) was inserted immediately downstream of the LoxP site inserted at position 497 to create a second recognition site for the restriction enzyme FseI. An inverted recombinase-flanked packing sequence was generated by inversion of a sequence comprising the recombinase-flanked packaging sequence. The inverted sequence for Construct 8 includes the sequence flanked by the two FseI sites at positions 143 and 497-which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 95:1574-1584 (2014). FIG. 9D shows a region of an Ad35 helper genome that includes Construct 8. Sequence information is provided below.

### Sequence Corresponding to AY128640 Nucleotides 1-497 Engineered to Include an Inverted Recombinase-Flanked Packing Sequence (Inverted Sequence Underlined; FseI Recognition Sites Bold and Italicized)

(SEQ ID NO: 40)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTTTTATGGCCGG
CCATAACTTCGTATAATGTATGCTATACGAAGTTATATTACCCTGTTAT
CCCTAAATACCCTAGCGATCAGCTGACACCTACGTAAAAACAGAAGACT
TTGACACGGTACGCGGAAATTCAGGTAAAAAACACGGTAATCGAAACCT
CCACGTAATGGGTCAAAGTCTACCTGGCCCTGAACAAATACTCCACCCT
GCCATAAATACCACATTATTCAGAAAAACACTTCCTCATTCAGTTTTCG
CGCGAAAATCAGCAATTTTCACTTGCATCCGGTCAAAACTACCTCATTT
CCTGTTAAATACCGTGGGAAAACTAAGTAGTTCCGTGAGAAAAGAAGCC
TTTTTATGCGTAACATTTCCCGCGACAACTATAACTTCGTATAATGTAT
GCTATACGAAGTTATTGCAAAAAAACTCCACCCGGCCGGCC

Components of the above sequence are further described below.

### ITR (CTATCTAT (SEQ ID NO: 12) Sequence Used in Place of the Canonical CATCATCA (SEQ ID NO: 13) Underlined)

(SEQ ID NO: 17)
CTATCTATATAATATACCTTATAGATGGAATGGTGCCAATATGTAAATG
AGGTGATTTTAAAAAGTGTGGGCCGTGTGGTGATTGGCTGTGGGGTTAA
CGGTTAAAAGGGGCGGCGCGGCCGTGGGAAAATGACGTT

### Inverted LoxP Sequence

(SEQ ID NO: 1)
ATAACTTCGTATAATGTATGCTATACGAAGTTAT

### Inverted Sequence (Inverted LoxP Sequences Underlined; Inverted Recombinase-Flanked Packaging Sequence Bold and Italicized)

(SEQ ID NO: 41)
ATAACTTCGTATAATGTATGCTATACGAAGTTATATTACCCTGTTATCC
CTAAATACCCTAGCGATCAGCTGACACCTACGTAAAAACAGAAGACTTT
GACACGGTACGCGGAAATTCAGGTAAAAAACACGGTAATCGAAACCTCC
ACGTAATGGGTCAAAGTCTACCTGGCCCTGAACAAATACTCCACCCTGC
CATAAATACCACATTATTCAGAAAAACACTTCCTCATTCAGTTTTCGCG
CGAAAATCAGCAATTTTCACTTGCATCCGGTCAAAACTACCTCATTTCC
TGTTAAATACCGTGGGAAAACTAAGTAGTTCCGTGAGAAAAGAAGCCTT
TTTATGCGTAACATTTCCCGCGACAACTATAACTTCGTATAATGTATGC
TATACGAAGTTATTGCAAAAAAACTCCACCC

### Inverted Recombinase-Flanked Packaging Sequence (Identified Inverted Packaging Signals A1, A2, A5 and A6 Underlined)

(SEQ ID NO: 42)
ATTACCCTGTTATCCCTAAATACCCTAGCGATCAGCTGACACCTACGTA
AAAACAGAAGACTTTGACACGGTACGCGGAAATTCAGGTAAAAAACACG
GTAATCGAAACCTCCACGTAATGGGTCAAAGTCTACCTGGCCCTGAACA
AATACTCCACCCTGCCATAAATACCACATTATTCAGAAAAACACTTCCT
CATTCAGTTTTCGCGCGAAAATCAGCAATTTTCACTTGCATCCGGTCAA
AACTACCTCATTTCCTGTTAAATACCGTGGGAAAACTAAGTAGTTCCGT
GAGAAAAGAAGCCTTTTTATGCGTAACATTTCCCGCGACAACT

### Example 6: Analysis of Ad35 Helper Genome Propagation and Stability

The present Example demonstrates successful use of Ad35 helper genomes as disclosed herein that include an inverted packaging sequence. The present Example further demonstrates that Ad35 helper genomes including inverted, recombinase-flanked packaging sequences according to the present disclosure are stable and can be propagated without detectable genome rearrangement.

Broadly, a helper genome can be present in a plasmid or in a viral vector. Plasmid forms can be used to transfect target cells for production of helper vectors (which helper vectors include the Ad35 helper genome) or for production of donor vectors (which donor vectors do not include the Ad35 helper genome). In the present Example, two plasmids encoding E1-deleted Ad35 helper genomes (designated pEN0056 and pEN0057), were each transfected into HEK293 cells and propagated to determine whether viable helper viruses could be rescued. Each of pEN0056 and pEN0057 included a construct according to Constructs 7 and 8 in Example 5, respectively.

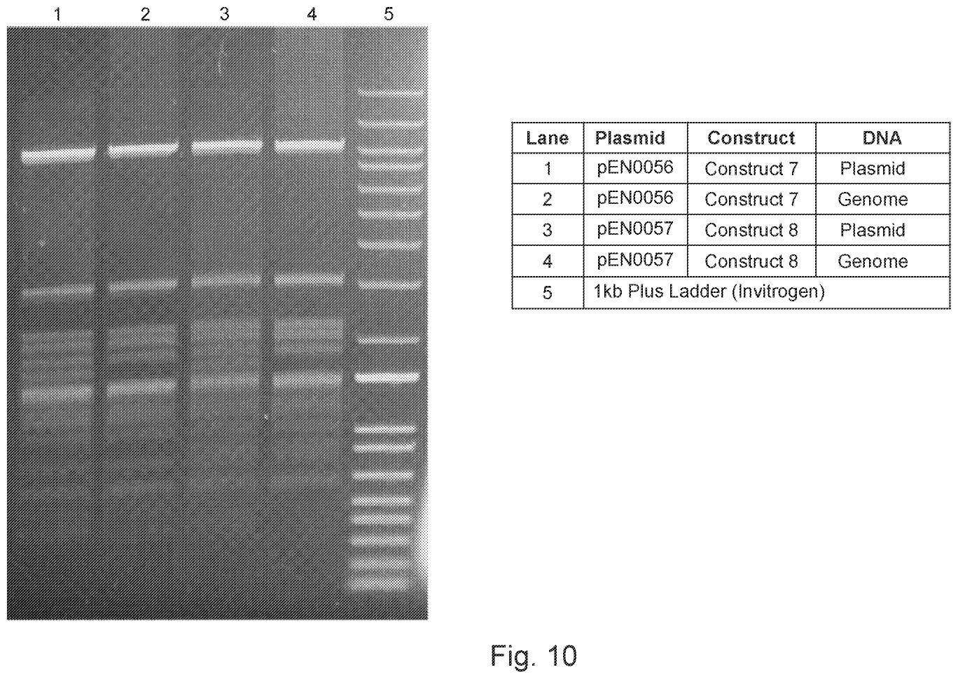

Rescued E1-deleted adenoviruses were purified using standard methods (see, e.g., Su et al. doi:10.1101/pdb.prot095547 Cold Spring Harb Protoc 2019) and viral genomes were isolated from purified helper vectors. Isolated Ad35 helper genomes were digested with XmnI alone, and starting plasmids were digested with XmnI and SwaI (which excises the plasmid backbone sequence) for comparison. Digestion products were analyzed by gel electrophoresis (FIG. 10).

To determine whether the Ad35 helper genomes were stable during propagation the restriction patterns obtained by digesting isolated adenoviral genomic DNA were compared to the restriction patterns obtained by digesting starting plasmids with the restriction enzymes XmnI and SwaI. Analysis of the restriction patterns on a gel showed the expected banding pattern and expected band sizes (FIG. 10), demonstrating that that Ad35 helper genomes including inverted recombinase-flanked packaging sequences as disclosed herein are genetically stable and can be propagated without detectable genome rearrangement in large-scale preparations.

### Example 7: Analysis of Recombinase-Mediated Excision of Inverted Recombinase-Flanked Packaging Sequences in Ad35 Helper Genomes

The present Example demonstrates the recombinase-mediated deletion of inverted recombinase-flanked packaging sequences in Ad35 helper genomes. Plasmids including Ad35 helper genomes (pEN0056 and pEN0057) were linearized by digestion with SwaI (which excised the plasmid backbone sequence) and transfected into each of two cell types: HEK293 cells that do not express Cre recombinase, and 116 cells modified from HEK293 cells to express Cre recombinase. Thus, excision of loxP flanked sequences is expected in the 116 cells but not the HEK293 cells. DNA was isolated from transfected cells and digested with the restriction enzyme ApaI. Digestion of the Ad35 helper genome with restriction enzyme ApaI is expected to produce a 2013 bp fragment. A smaller DNA fragment is expected if the Ad35 helper genome has undergone recombination to mediate deletion of the inverted recombinase-flanked packaging sequence. Restriction results were analyzed by gel electrophoresis (FIG. 11). The expected band sizes were observed for DNA isolated from HEK293 cells transfected with the Ad35 helper genomes (FIG. 11—lanes 2 and 4) and for DNA isolated from 116 cells transfected with the Ad35 helper genomes (FIG. 11—lanes 3 and 5). Data therefore show successful Cre-mediated excision of flanked packaging sequences from all helper genomes in the presence of recombinase.

### Example 8: Analysis of Helper-Dependent Adenovirus (HDAd) Production Using Ad35 Helper Vectors with Genomes Including Inverted Recombinase-Flanked Packaging Sequences

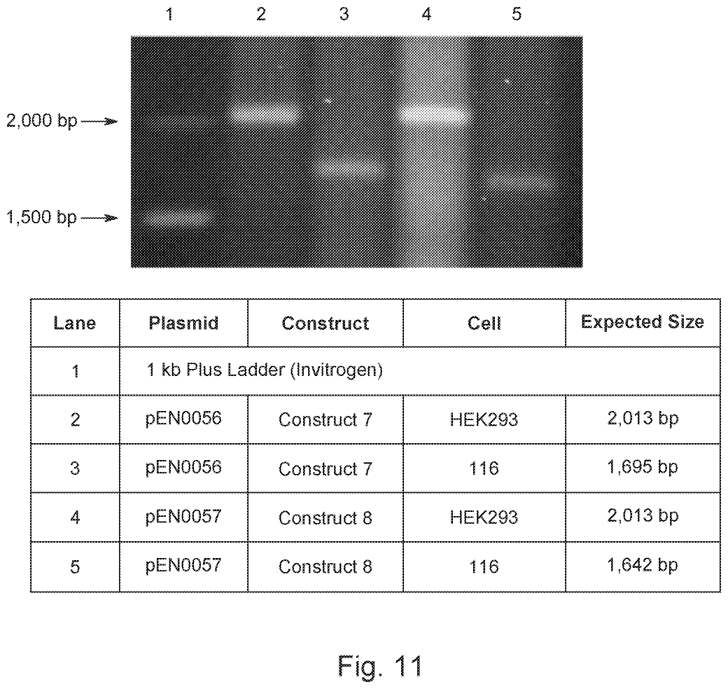

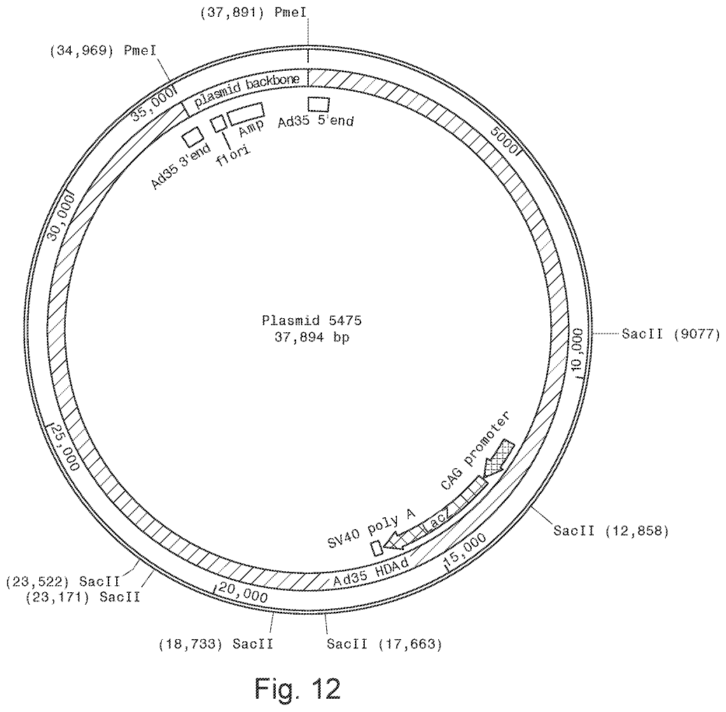

The present Example demonstrates the production of helper-dependent adenovirus (HDAd) using Ad35 helper vectors with genomes including inverted recombinase-flanked packaging sequences. Ad35 helper vectors were purified from HEK293 cells transfected with plasmids including Ad35 helper genomes with inverted recombinase-flanked packaging sequences (pEN0056 and pEN0057). Helper-dependent adenoviral vectors were produced according to standard procedures (see Palmer and Ng, Methods Mol Biol. 2008; 433:33-53) in 116 cells using the purified Ad35 helper vectors and transfecting plasmid 5475, a plasmid that encodes a helper-dependent genome that includes terminal sequences derived from Ad35 and includes a cassette for expression of beta-galactosidase (FIG. 12). HDAd viral particles produced using Ad35 helper vectors from pEN0056 and pEN0057 were isolated and subsequently used to achieve production of secondary HDAd preparations by co-infection of 116 cells with the HDAd viral particles from plasmid 5475 and Ad35 helper viral particles from pEN0056 and pEN0057 (respectively).

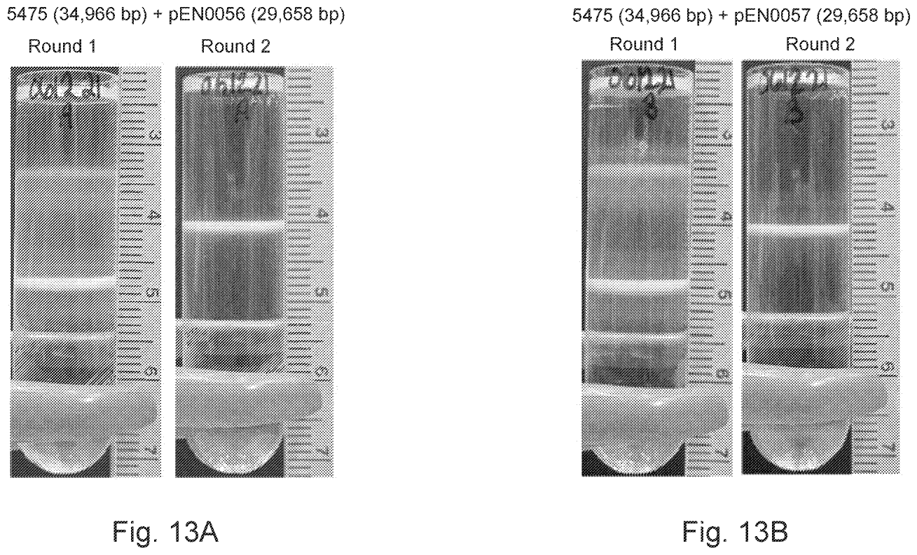

Helper-dependent adenovirus (HDAd) preparations were purified by using two consecutive cesium chloride continuous gradients (FIG. 13A-B). Purified HDAd preparations were characterized using several approaches. The physical titer or yield of the purified virus preparations was determined by spectrophotometry and can be expressed as the total number of purified viral particles (vp) or the number of viral particles per volume (vp/ml). The infectivity of the purified HDAd preparations was determined by using the purified helper-dependent viruses to infect cultured HEK293 cells and staining the cells to determine their expression of beta-galactosidase (as described in Parks et al., PNAS. 1996: 93(24):13565-13570). Infected cells were expected to express beta-galactosidase. Infectivity was represented in terms of blue-forming units (BFU), which is the number of cells that showing blue staining indicating positive expression of beta-galactosidase encoded by the cassette in HDAd genome. Infectivity can be further represented as the BFU per volume of purified virus (BFU/ml) and/or the ratio between the total number of viral particles and the BFU (vp:BFU).

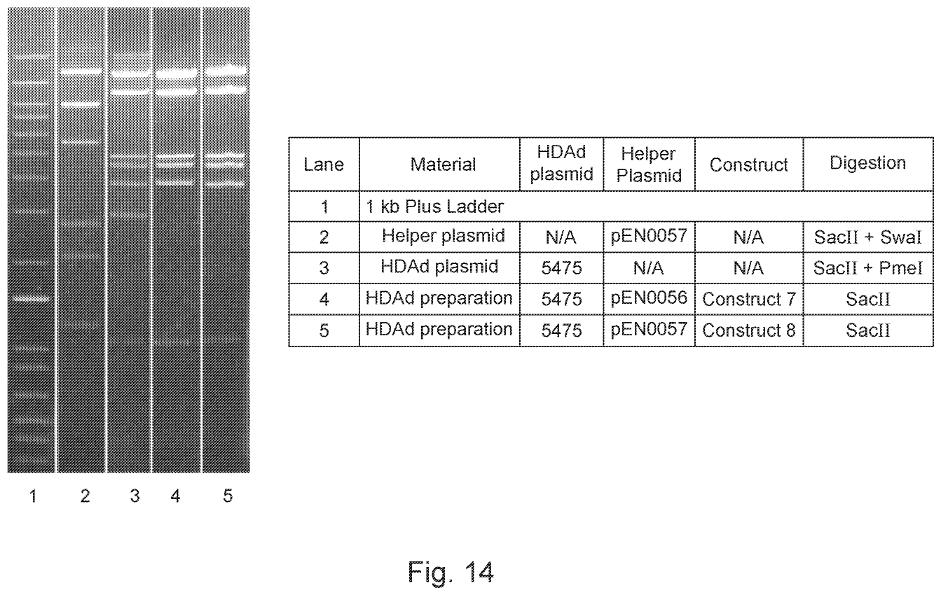

Further characterization of the purified HDAd preparations was performed using DNA isolated from the purified HDAd preparations. Isolated DNA was digested using restriction enzyme (SacII) and the restriction pattern was compared to the restriction pattern obtained by digestion using restriction enzymes (SacII and PmeI) of the starting HDAd plasmid and the restriction pattern obtained by digestion using restriction enzymes (SacII and SwaI) of the starting Ad35 helper plasmids. Analysis of the restriction patterns on a gel showed the expected banding pattern and expected band sizes (FIG. 14), indicating successful HDAd production. Vectors, genomes, and conditional packaging sequences analyzed in FIG. 14 are advantageous and useful for various methods and compositions provided herein. Additionally, the Ad35 helper contamination fraction in the purified preparation was determined using quantitative PCR of DNA isolated from the purified HDAd preparation.

Table 38 shows the results from experiments to characterize the purified HDAd preparations. Table 39 shows results from secondary preparations, including estimated helper fraction (%).

TABLE 38
Characterization of Purified HDAd Preparations

| Helper | | Yield | Yield | Infectivity | Infectivity | Helper fraction |
|---|---|---|---|---|---|---|
| plasmid | Construct | (vp) | (vp/ml) | (BFU/ml) | (vp:BFU) | (%) |
| pEN0056 | Construct 7 | 3.23e12 | 1.126e12 | 5.69e10 | 19.8:1 | 1.44 |
| pEN0057 | Construct 8 | 2.45e12 | 7.806e11 | 2.44e10 | 32:1 | 1.67 |

TABLE 39
Characterization of Secondary HDAd Preparations

| | | | | Helper |
|---|---|---|---|---|
| Helper | | Yield | Infectivity | fraction |
| plasmid | Construct | (vp) | (vp:BFU) | (%) |
| pEN0057 | Construct 8 | 5e12 | 17.6:1 | 0.95 |

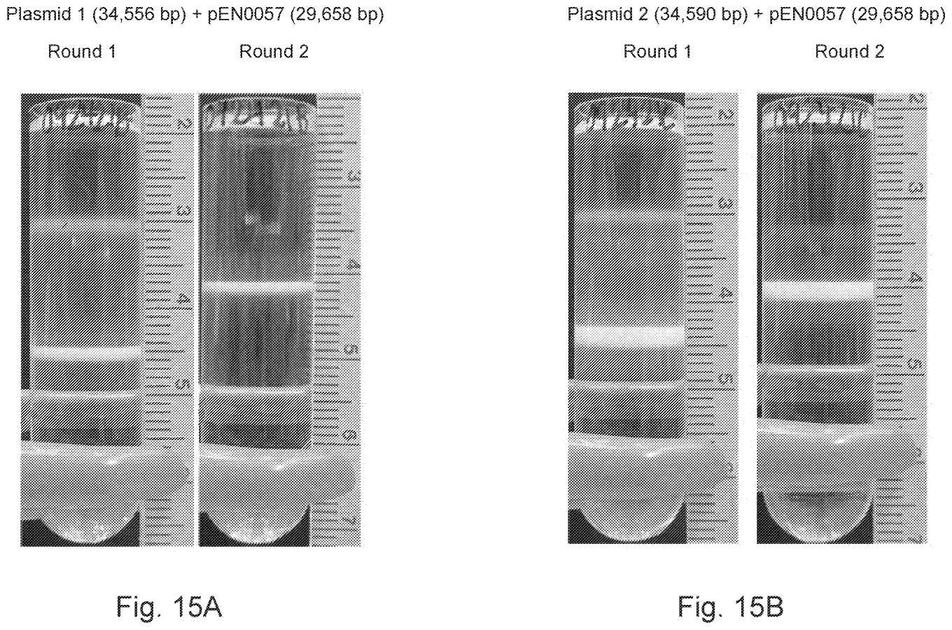

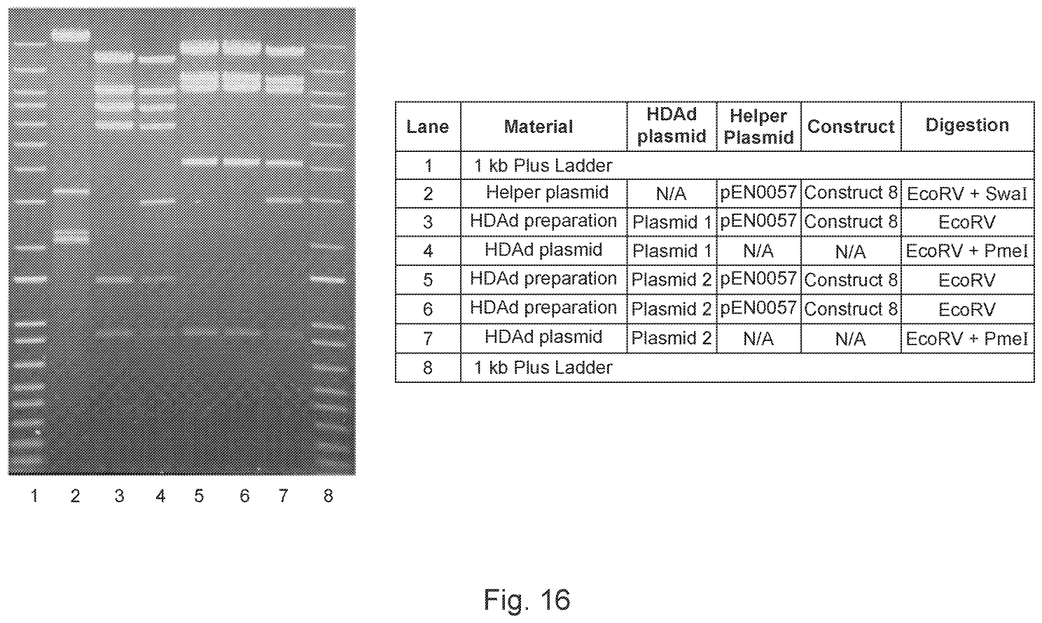

To further demonstrate production of helper-dependent adenovirus (HDAd) using Ad35 helper vectors with genomes including inverted, recombinase-flanked packaging sequences, HDAd vectors were produced in 116 cells using a purified Ad35 helper vector (from pEN0057) and transfecting one of two exemplary plasmids (plasmid 1 and plasmid 2). Plasmid 1 and plasmid 2 encode exemplary helper-dependent genomes that each include terminal sequences derived from Ad35 and includes an exemplary transgene payload, each of plasmids 1 and 2 including a different exemplary transgene payload. HDAd preparations were purified by using two consecutive cesium chloride continuous gradients (FIG. 15A-B). Purified HDAd preparations were characterized as described above. DNA isolated from the purified HDAd preparations was digested using restriction enzyme (EcoRV) and the restriction pattern was compared to the restriction pattern obtained by digestion using restriction enzymes (EcoRV and PmeI) of the starting HDAd plasmids and the restriction pattern obtained by digestion using restriction enzymes (EcoRV and SwaI) of the starting Ad35 helper plasmid. Analysis of the restriction patterns on a gel showed the expected banding pattern and expected band sizes (FIG. 16), indicating successful HDAd production.

Table 40 shows the results from experiments to characterize the purified HDAd preparations.

TABLE 40
Characterization of Purified HDAd Preparations

| | | | | Helper |
|---|---|---|---|---|
| Helper | | HDAd | Yield | fraction |
| plasmid | Construct | plasmid | (vp) | (%) |
| pEN0057 | Construct 8 | Plasmid 1 | 2.08e12 | 0.75 |
| pEN0057 | Construct 8 | Plasmid 2 | 4.27e12 | 0.75 |
| pEN0057 | Construct 8 | Plasmid 2 | 3.74e12 | 0.81 |

### Example 9: Design of Helper Genomes

The present Example includes the identification of positions within Ad3, Ad7, Ad11, Ad14, Ad16, Ad21, Ad34, and Ad50 genomes, and particularly within the Ad3, Ad7, Ad11, Ad14, Ad16, Ad21, Ad34, and Ad50 packaging sequences, at which recombinase sites can be positioned for efficient switching between packaging competence and packaging deficiency. A person of skill in the art will appreciate that data presented in the preceding examples for Ad35 can be extrapolated to other species B serotypes including Ad3, Ad7, Ad11, Ad14, Ad16, Ad21, Ad34 and Ad50, based at least in part on the recognized functional and sequence similarity of serotypes within the species. Insertion or positioning of the recombinase sites into the packaging sequence will not abrogate adenoviral genome packaging. However, excision of the recombinase-flanked sequences will reduce and/or eliminates packaging of the genome.

The present Example includes alignment of adenoviral packaging sequences to identify putative packaging signals in the Ad3, Ad7, Ad11, Ad14, Ad16, Ad21, Ad34, and Ad50 genomes, as shown in FIG. 1B. The present Example further includes selection of particular locations for placement of 5′ (left) and 3′ (right) recombinase sites, also shown in FIG. 1B and Table 21. Selected left recombinase sites include positions corresponding to an L1, L2, L3, or L4 site of a reference Ad3, Ad7, Ad11, Ad14, Ad16, Ad21, Ad34, or Ad50 genome as disclosed in Table 21. Selected right recombinase sites include positions corresponding to an R1, R2, or R3 site of a reference Ad3, Ad7, Ad11, Ad14, Ad16, Ad21, Ad34, or Ad50 genome as disclosed in Table 21. The present Example further includes particular pairings of left and right recombinase site positions: position L1 with position R3, position L2 with position R1, position L3 with position R2, and position L4 with position R1. These pairing are shown as exemplary Constructs 9-40 in Table 41.

TABLE 41
Exemplary constructs including recombinase-flanked packaging sequences

| | | Left | Right | |
|---|---|---|---|---|
| Exemplary | | Recombinase | Recombinase | |
| Construct | Serotype | Position | Position | Reference Sequence |
| 9 | Ad3 | L1 (163) | R3 (500) | NCBI accession no. NC_011203 |
| 10 | Ad3 | L2 (173) | R1 (405) | NCBI accession no. NC_011203 |
| 11 | Ad3 | L3 (197) | R2 (482) | NCBI accession no. NC_011203 |
| 12 | Ad3 | L4 (227) | R1 (405) | NCBI accession no. NC_011203 |
| 13 | Ad7 | L1 (163) | R3 (500) | GenBank accession no. AY601634 |
| 14 | Ad7 | L2 (173) | R1 (405) | GenBank accession no. AY601634 |
| 15 | Ad7 | L3 (197) | R2 (482) | GenBank accession no. AY601634 |
| 16 | Ad7 | L4 (227) | R1 (405) | GenBank accession no. AY601634 |
| 17 | Ad11 | L1 (161) | R3 (498) | NCBI accession no. NC_011202 |
| 18 | Ad11 | L2 (171) | R1 (401) | NCBI accession no. NC_011202 |
| 19 | Ad11 | L3 (195) | R2 (478) | NCBI accession no. NC_011202 |
| 20 | Ad11 | L4 (223) | R1 (401) | NCBI accession no. NC_011202 |
| 21 | Ad14 | L1 (161) | R3 (498) | GenBank accession no. AY803294 |
| 22 | Ad14 | L2 (171) | R1 (419) | GenBank accession no. AY803294 |
| 23 | Ad14 | L3 (195) | R2 (496) | GenBank accession no. AY803294 |
| 24 | Ad14 | L4 (223) | R1 (419) | GenBank accession no. AY803294 |
| 25 | Ad16 | L1 (161) | R3 (498) | GenBank accession no. AY601636 |
| 26 | Ad16 | L2 (171) | R1 (403) | GenBank accession no. AY601636 |
| 27 | Ad16 | L3 (195) | R2 (480) | GenBank accession no. AY601636 |
| 28 | Ad16 | L4 (225) | R1 (403) | GenBank accession no. AY601636 |
| 29 | Ad21 | L1 (161) | R3 (498) | GenBank accession no. AY601633 |
| 30 | Ad21 | L2 (171) | R1 (403) | GenBank accession no. AY601633 |
| 31 | Ad21 | L3 (195) | R2 (480) | GenBank accession no. AY601633 |
| 32 | Ad21 | L4 (225) | R1 (403) | GenBank accession no. AY601633 |
| 33 | Ad34 | L1 (161) | R3 (498) | GenBank accession no. AY737797 |
| 34 | Ad34 | L2 (171) | R1 (402) | GenBank accession no. AY737797 |
| 35 | Ad34 | L3 (195) | R2 (479) | GenBank accession no. AY737797 |
| 36 | Ad34 | L4 (224) | R1 (402) | GenBank accession no. AY737797 |
| 37 | Ad50 | L1 (160) | R3 (497) | GenBank accession no. AY737798 |
| 38 | Ad50 | L2 (170) | R1 (402) | GenBank accession no. AY737798 |
| 39 | Ad50 | L3 (194) | R2 (479) | GenBank accession no. AY737798 |
| 40 | Ad50 | L4 (224) | R1 (402) | GenBank accession no. AY737798 |

Constructs 9-40 are presented in Table 41 and include recombinase sites positions to generate a conditionally defective packaging sequence of an adenoviral genome. For each construct, positions of inserted LoxP sites are indicated relative to a respective adenoviral genome sequence. The LoxP sites flank an adenoviral packaging sequence so that Cre recombinase-mediated deletion of the flanked sequences will render the genome deficient for packaging. The inserted LoxP sequence corresponds to ATAACTTCGTATAGCATACATTATACGAAGTTAT (SEQ ID NO: 18). Optionally, in the 5′ inverted terminal repeat (ITR) of the indicated genome, CTATCTAT (SEQ ID NO: 12) can be used in place of the canonical sequence present at positions 1-8 in the indicated reference sequence, based on the publication by Wunderlich et al., J Gen Virol. 95:1574-1584 (2014).

### Example 10: Design of Helper Genomes Including Inverted Packaging Sequences

The present Example demonstrates the design of Ad3, Ad7, Ad11, Ad14, Ad16, Ad21, Ad34, and Ad50 helper genomes that include inverted packaging sequences. The present Example is based at least in part on the recognition that use of an inverted packaging sequence in an Ad3, Ad7, Ad11, Ad14, Ad16, Ad21, Ad34, or Ad50 helper vector can reduce and/or eliminate recombinase site-excising homologous recombination (compare FIG. 8A and FIG. 8B). Inversion of sequences comprising a conditionally defective packaging sequence-thereby generating an inverted recombinase-flanked packaging sequences-will reduce and/or eliminate recombinase site-excising homologous recombination, as shown in FIG. 8B. Sequence elements included within an inverted sequence are herein referred to as inverted sequence elements (e.g., a recombinase-flanked packaging sequence included within an inverted sequence is referred to as an inverted recombinase-flanked packaging sequence). A person of skill in the art would appreciate from the present disclosure that the orientation of the packaging sequence is not critical to its function (see, e.g., Palmer and Ng, Mol Ther. 2003; 8:8460852) and would further appreciate from the present disclosure that an inverted conditionally defective packaging sequence as disclosed herein is packaging competent. An inverted recombinase site flanked packaging sequence can further be excised by recombination upon contact with a corresponding recombinase and prevent packaging of a helper genome.

The present Example particularly includes Ad3, Ad7, Ad11, Ad14, Ad16, Ad21, Ad34, and Ad50 helper genomes including inverted packaging sequences, as shown as exemplary Constructs 41-72 in Table 42. Constructs 41-72 correspond to Constructs 9-41 but includes a packaging sequence inversion. Constructs 41-72 contain loxP sites inserted at the positions indicated for Constructs 9-40 as indicated in Table 41. Positions of the inserted loxP sequences are identified based on their correspondence with positions in the indicated reference genome sequence, regardless of whether the position is present in an inverted orientation. The inserted loxP sites in each of Constructs 41-72 flank a packaging sequence of the corresponding genome, so that Cre recombinase-mediated deletion of the flanked packaging sequence will render the genome deficient for packaging. An inverted recombinase-flanked packaging sequence is generated by inversion of a sequence comprising the recombinase-flanked packaging sequence. The inverted sequence for each of Constructs 41-72 includes a sequence have a first end point and a second endpoint corresponding to a left inversion position (Left Inversion Point) and right inversion position (Right Inversion Point) of a corresponding reference genome, as set forth in Table 28. The inverted sequences include the two loxP sites and the recombinase-flanked packaging sequence. The inverted LoxP sites flank the inverted recombinase-flanked packaging sequence of the Ad3, Ad7, Ad11, Ad14, Ad16, Ad21, Ad34, or Ad50 genome so that Cre recombinase-mediated deletion of the flanked sequences will render the genome deficient for packaging. Optionally, in the 5′ inverted terminal repeat (ITR) of the indicated genome, CTATCTAT (SEQ ID NO: 12) can be used in place of the canonical sequence present at positions 1-8 in the indicated reference sequence, based on the publication by Wunderlich et al., J Gen Virol. 95:1574-1584 (2014).

TABLE 42
Exemplary constructs including inverted recombinase-flanked packaging sequences

| | | | Left | Right | |
|---|---|---|---|---|---|
| Exemplary | | Related | Inversion | Inversion | |
| Construct | Serotype | Construct | Position | Position | Reference Sequence |
| 41 | Ad3 | 9 | 137 | 500 | NCBI accession no. NC_011203 |
| 42 | Ad3 | 10 | 137 | 500 | NCBI accession no. NC_011203 |
| 43 | Ad3 | 11 | 137 | 500 | NCBI accession no. NC_011203 |
| 44 | Ad3 | 12 | 137 | 500 | NCBI accession no. NC_011203 |
| 45 | Ad7 | 13 | 137 | 500 | GenBank accession no. AY601634 |
| 46 | Ad7 | 14 | 137 | 500 | GenBank accession no. AY601634 |
| 47 | Ad7 | 15 | 137 | 500 | GenBank accession no. AY601634 |
| 48 | Ad7 | 16 | 137 | 500 | GenBank accession no. AY601634 |
| 49 | Ad11 | 17 | 138 | 498 | NCBI accession no. NC_011202 |
| 50 | Ad11 | 18 | 138 | 498 | NCBI accession no. NC_011202 |
| 51 | Ad11 | 19 | 138 | 498 | NCBI accession no. NC_011202 |
| 52 | Ad11 | 20 | 138 | 498 | NCBI accession no. NC_011202 |
| 53 | Ad14 | 21 | 138 | 498 | GenBank accession no. AY803294 |
| 54 | Ad14 | 22 | 138 | 498 | GenBank accession no. AY803294 |
| 55 | Ad14 | 23 | 138 | 498 | GenBank accession no. AY803294 |
| 56 | Ad14 | 24 | 138 | 498 | GenBank accession no. AY803294 |
| 57 | Ad16 | 25 | 115 | 498 | GenBank accession no. AY601636 |
| 58 | Ad16 | 26 | 115 | 498 | GenBank accession no. AY601636 |
| 59 | Ad16 | 27 | 115 | 498 | GenBank accession no. AY601636 |
| 60 | Ad16 | 28 | 115 | 498 | GenBank accession no. AY601636 |
| 61 | Ad21 | 29 | 115 | 498 | GenBank accession no. AY601633 |
| 62 | Ad21 | 30 | 115 | 498 | GenBank accession no. AY601633 |
| 63 | Ad21 | 31 | 115 | 498 | GenBank accession no. AY601633 |
| 64 | Ad21 | 32 | 115 | 498 | GenBank accession no. AY601633 |
| 65 | Ad34 | 33 | 138 | 498 | GenBank accession no. AY737797 |
| 66 | Ad34 | 34 | 138 | 498 | GenBank accession no. AY737797 |
| 67 | Ad34 | 35 | 138 | 498 | GenBank accession no. AY737797 |
| 68 | Ad34 | 36 | 138 | 498 | GenBank accession no. AY737797 |
| 69 | Ad50 | 37 | 115 | 497 | GenBank accession no. AY737798 |
| 70 | Ad50 | 38 | 115 | 497 | GenBank accession no. AY737798 |
| 71 | Ad50 | 39 | 115 | 497 | GenBank accession no. AY737798 |
| 72 | Ad50 | 40 | 115 | 497 | GenBank accession no. AY737798 |

### Example 11: Analysis of Helper Genome Propagation and Stability

The present Example includes demonstration that helper genomes including recombinase-flanked packaging sequences and helper genomes including an inverted packaging sequence (e.g., an inverted, recombinase-flanked packaging sequence) according to the present disclosure are stable and can be propagated without detectable genome rearrangement.

Broadly, a helper genome can be present in a plasmid or in a viral vector. Plasmid forms can be used to transfect target cells for production of helper vectors (which helper vectors include the helper genome) or for production of donor vectors (which donor vectors do not include the helper genome). In the present Example, plasmids encoding exemplary E1-deleted helper genomes including a construct according to Examples 9 and 10 are each transfected into HEK293 cells and propagated to determine whether viable helper viruses can be rescued.

Rescued E1-deleted adenoviruses are purified using standard methods (see, e.g., Su et al., Cold Spring Harb Protoc. (2019), doi:10.1101/pdb.prot095547) and viral genomes are isolated from purified helper vector. Isolated helper genomes and starting plasmids are digested with restriction enzymes. To determine whether the helper genomes are stable during propagation, restriction patterns obtained by digesting isolated adenoviral genomic DNA are compared to the restriction patterns obtained by digesting starting plasmids. Analysis of the restriction patterns by gel electrophoresis will demonstrate that helper genomes including recombinase-flanked packaging sequences and helper genomes including inverted recombinase-flanked packaging sequences are genetically stable and can be propagated without detectable genome rearrangement in large-scale preparations.

### Example 12: Analysis of Recombinase-Mediated Excision of Recombinase-Flanked Packaging Sequences in Helper Genomes

The present Example includes demonstration of recombinase-mediated deletion of recombinase-flanked packaging sequences and inverted, recombinase-flanked packing sequences in helper genomes. Plasmids encoding exemplary E1-deleted helper genomes including a construct according to Examples 9 and 10 are linearized by restriction enzyme digestion (which excises the plasmid backbone sequence) and transfected into each of two cell types: HEK293 cells that do not express Cre recombinase, and 116 cells modified from HEK293 cells to express Cre recombinase. Accordingly, excision of loxP-flanked sequences is expected in the 116 cells but not the HEK293 cells. DNA is isolated from transfected cells and subject to restriction enzyme digestion. Digestion of the helper genome is expected to produce different restriction patterns depending on whether the helper genome has undergone recombination to mediate deletion of the recombinase-flanked packaging sequence or the inverted, recombinase-flanked packing sequence. A person of skill in the art would appreciate that the expected restriction patterns can be readily determined based on the sequence of the helper genome and the particular restriction enzyme(s) being used for digestion. Restriction results are analyzed by gel electrophoresis. Observation of the expected band sizes will show successful Cre-mediated excision of flanked packaging sequences from the helper genomes in the presence of recombinase.

### Example 13: Analysis of Helper-Dependent Adenovirus (HDAd) Production Using Helper Vectors with Genomes Including Recombinase-Flanked Packaging Sequences

The present Example includes demonstration of the production of helper-dependent adenovirus (HDAd) using helper vectors with genomes including recombinase-flanked packaging sequences or inverted recombinase-flanked packaging sequences. Helper vectors are purified from HEK293 cells transfected with plasmids exemplary E1-deleted helper genomes including a construct according to Examples 9 and 10. Helper-dependent adenoviral vectors are then produced according to standard procedures (see, Palmer and Ng, Methods Mol Biol. 433:33-53 (2008)) in 116 cells using the purified helper vectors and transfecting a plasmid that encodes a helper-dependent genome that includes (i) terminal sequences derived from the same adenoviral serotype as the helper vector and (ii) a cassette for expression of a reporter gene (e.g., beta-galactosidase). HDAd viral particles produced using helper vectors are isolated and subsequently used to produce secondary HDAd preparations by co-infection of 116 cells with the HDAd viral particles and helper viral particles.

HDAd preparations are purified (e.g., by using two consecutive cesium chloride continuous gradients). Purified HDAd preparations are characterized using several approaches. The physical titer or yield of the purified virus preparations is determined by spectrophotometry and can be expressed as the total number of purified viral particles (vp) or the number of viral particles per volume (vp/ml). The infectivity of the purified HDAd preparations is determined by using the purified helper-dependent viruses to infect cultured HEK293 cells and analyzing the cells to determine their expression of the reporter gene (e.g., by staining the cells to determine their expression of beta-galactosidase, as described in Parks et al., PNAS. 93(24):13565-13570 (1996)). Infected cells are expected to express the reporter gene. Infectivity can be expressed as the number of cells showing expression of the reporter gene (e.g., in terms of blue-forming units (BFU) for beta-galactosidase as a reporter gene). Infectivity can be further represented as the number of cells showing expression of the reporter gene per volume of purified virus and/or the ratio between the total number of viral particles and the number of cells showing expression of the reporter gene.

Further characterization of the purified HDAd preparations was performed using DNA isolated from the purified HDAd preparations. Further characterization of the purified HDAd preparations is performed using DNA isolated from the purified HDAd preparations. Isolated DNA is digested using restriction enzyme(s) and the restriction pattern is compared to (i) the restriction pattern obtained by digestion using restriction enzyme(s) of the starting HDAd plasmid and (ii) the restriction pattern obtained by digestion using restriction enzyme(s) of the starting helper plasmids. Analysis of the restriction patterns on a gel will show the expected banding pattern and expected band, indicating successful HDAd production. Additionally, the helper contamination faction in the purified preparation is determined using quantitative PCR of DNA isolated from the purified HDAd preparation.

### Accession Sequences

Provided herein is a listing of nucleic acid sequences and amino acid sequences corresponding to publicly available sequence accession numbers, certain of which sequences and/or sequence accession numbers are included and/or utilized, in whole and/or in part, in the present disclosure, and/or certain of which sequences and/or sequence accession numbers are included herein as references. Sequences associated with accession numbers are available in publicly accessible databases, as is known to those of skill in the art, and such sequences are provided herein solely for easy for reference.

### OTHER EMBODIMENTS

While we have described a number of embodiments, it is apparent that our disclosure and examples also provide other embodiments that utilize or are encompassed by the compositions and methods described herein. Therefore, it will be appreciated that the scope of disclosure is to be defined by that which may be understood from the disclosure rather than by the specific embodiments that have been represented by way of example. Limitations described with respect to one aspect of the disclosure, in certain embodiments, be practiced with respect to other aspects of the disclosure. For example, limitations of claims that depend directly or indirectly from a certain independent claim presented herein serve as support for those limitations being presented in additional dependent claims of one or more other independent claims.

All references cited herein are hereby incorporated by reference.

What is claimed is:

1. A recombinant adenoviral helper genome comprising:
a 5′ inverted terminal repeat (ITR);
a 3′ ITR; and
a packaging sequence;
wherein the 5′ ITR, the 3′ ITR, and the packaging sequence are each derived from a species B adenovirus of a serotype selected from Ad3, Ad7, Ad11, Ad14, Ad16, Ad21, Ad34, or Ad50;
wherein the packaging sequence is flanked by or comprises recombinase direct repeats comprising a first recombinase direct repeat and a second recombinase direct repeat;
wherein the position of the first recombinase direct repeat corresponds to a position that is within 10 nucleotides of an L1, L2, L3, or L4 site of a reference sequence for the serotype of the packaging sequence, as set forth in Table 21; and
wherein the position of the second recombinase direct repeat corresponds to a position that is within 10 nucleotides of an R1, R2, or R3 site of a reference sequence for the serotype of the packaging sequence, as set forth in Table 21.

2. The helper genome of claim 1, wherein the position of the first recombinase direct repeat corresponds to a position that is within 10 nucleotides of an L1 site of a reference sequence for the serotype of the packaging sequence, as set forth in Table 21; and wherein the position of the second recombinase direct repeat corresponds to a position that is within 10 nucleotides of an R3 site of a reference sequence for the serotype of the packaging sequence, as set forth in Table 21.

3. The helper genome of claim 1, wherein the position of the first recombinase direct repeat corresponds to a position that is within 10 nucleotides of an L2 site of a reference sequence for the serotype of the packaging sequence, as set forth in Table 21; and wherein the position of the second recombinase direct repeat corresponds to a position that is within 10 nucleotides of an R1 site of a reference sequence for the serotype of the packaging sequence, as set forth in Table 21.

4. The helper genome of claim 1, wherein the position of the first recombinase direct repeat corresponds to a position that is within 10 nucleotides of an L3 site of a reference sequence for the serotype of the packaging sequence, as set forth in Table 21; and wherein the position of the second recombinase direct repeat corresponds to a position that is within 10 nucleotides of an R2 site of a reference sequence for the serotype of the packaging sequence, as set forth in Table 21.

5. The helper genome of claim 1, wherein the position of the first recombinase direct repeat corresponds to a position that is within 10 nucleotides of an L4 site of a reference sequence for the serotype of the packaging sequence, as set forth in Table 21; and wherein the position of the second recombinase direct repeat corresponds to a position that is within 10 nucleotides of an R1 site of a reference sequence for the serotype of the packaging sequence, as set forth in Table 21.

6. The helper genome of claim 1, wherein the position of the first recombinase direct repeat corresponds to a position at an L1, L2, L3, or L4 site of a reference sequence for the serotype of the packaging sequence, as set forth in Table 21; and wherein the position of the second recombinase direct repeat corresponds to a position that is at an R1, R2, or R3 site of a reference sequence for the serotype of the packaging sequence, as set forth in Table 21.

7. The helper genome of claim 1, wherein the position of the first recombinase direct repeat corresponds to a position at an L1 site of a reference sequence for the serotype of the packaging sequence, as set forth in Table 21; and wherein the position of the second recombinase direct repeat corresponds to a position that is at an R3 site of a reference sequence for the serotype of the packaging sequence, as set forth in Table 21.

8. The helper genome of claim 1, wherein the position of the first recombinase direct repeat corresponds to a position at an L2 site of a reference sequence for the serotype of the packaging sequence, as set forth in Table 21; and wherein the position of the second recombinase direct repeat corresponds to a position that is at an R1 site of a reference sequence for the serotype of the packaging sequence, as set forth in Table 21.

9. The helper genome of claim 1, wherein the position of the first recombinase direct repeat corresponds to a position at an L3 site of a reference sequence for the serotype of the packaging sequence, as set forth in Table 21; and wherein the position of the second recombinase direct repeat corresponds to a position that is at an R2 site of a reference sequence for the serotype of the packaging sequence, as set forth in Table 21.

10. The helper genome of claim 1, wherein the position of the first recombinase direct repeat corresponds to a position at an L4 site of a reference sequence for the serotype of the packaging sequence, as set forth in Table 21; and wherein the position of the second recombinase direct repeat corresponds to a position that is at an R1 site of a reference sequence for the serotype of the packaging sequence, as set forth in Table 21.

11. The helper genome of claim 1, wherein the position of the first recombinase direct repeat corresponds to a position at an L1, L2, L3, or L4 site of a reference sequence for the serotype of the packaging sequence, as set forth in any one of Tables 22-27; and wherein the position of the second recombinase direct repeat corresponds to a position that is at an R1, R2, or R3 site of a reference sequence for the serotype of the packaging sequence, as set forth in any one of Tables 22-27.

12. The helper genome of claim 1, wherein the position of the first recombinase direct repeat corresponds to a position at an L1 site of a reference sequence for the serotype of the packaging sequence, as set forth in any one of Tables 22-27; and wherein the position of the second recombinase direct repeat corresponds to a position that is at an R3 site of a reference sequence for the serotype of the packaging sequence, as set forth in any one of Tables 22-27.

13. The helper genome of claim 1, wherein the position of the first recombinase direct repeat corresponds to a position at an L2 site of a reference sequence for the serotype of the packaging sequence, as set forth in any one of Tables 22-27; and wherein the position of the second recombinase direct repeat corresponds to a position that is at an R1 site of a reference sequence for the serotype of the packaging sequence, as set forth in any one of Tables 22-27.

14. The helper genome of claim 1, wherein the position of the first recombinase direct repeat corresponds to a position at an L3 site of a reference sequence for the serotype of the packaging sequence, as set forth in any one of Tables 22-27; and wherein the position of the second recombinase direct repeat corresponds to a position that is at an R2 site of a reference sequence for the serotype of the packaging sequence, as set forth in any one of Tables 22-27.

15. The helper genome of claim 1, wherein the position of the first recombinase direct repeat corresponds to a position at an L4 site of a reference sequence for the serotype of the packaging sequence, as set forth in any one of Tables 22-27; and wherein the position of the second recombinase direct repeat corresponds to a position that is at an R1 site of a reference sequence for the serotype of the packaging sequence, as set forth in any one of Tables 22-27.

16. The helper genome of any one of claims 1-15, wherein the 5′ ITR and the 3′ ITR are derived from the same serotype.

17. The helper genome of any one of claims 1-16, wherein the 5′ ITR, the 3′ ITR, and the packaging sequence are derived from the same serotype.

18. The helper genome of any one of claims 1-17, wherein the recombinase direct repeats that flank the packaging sequence are FRT, loxP, rox, vox, AttB, or AttP sites.

19. The helper genome of any one of claims 1-18, wherein the recombinase direct repeats that flank the packaging sequence are loxP sites.

20. A recombinant adenoviral helper vector comprising the helper genome of any one of claims 1-19.

21. A recombinant adenoviral vector production system comprising:
(i) the helper genome of any one of claims 1-19 or the helper vector of claim 20, and
(ii) a helper-dependent adenoviral (HDAd) donor genome, the HDAd donor genome comprising:
a 5′ inverted terminal repeat (ITR);
a 3′ ITR;
a packaging sequence; and
a nucleic acid sequence encoding at least one heterologous expression product;
wherein the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50.

22. A method of producing a recombinant helper-dependent adenoviral (HDAd) donor vector, the method comprising isolating the recombinant HDAd donor vector from a culture of cells, wherein the cells comprise:
a recombinant helper genome of any one of claims 1-19 or a recombinant adenoviral helper vector of claim 20; and
a recombinant HDAd donor genome comprising:
a 5′ inverted terminal repeat (ITR);
a 3′ ITR;
a packaging sequence; and
a nucleic acid sequence encoding at least one heterologous expression product;
wherein the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50.

23. The system or method of claim 21 or claim 22, wherein the 5′ ITR and the 3′ ITR of the HDAd donor genome are derived from the same serotype.

24. The system or method of any one of claims 21-23, wherein the 5′ ITR, the 3′ ITR, and the packaging sequence of the HDAd donor genome are derived from the same serotype.

25. The helper genome, helper vector, system, or method of any one of claims 1-24, wherein the helper genome comprises a nucleic acid sequence that encodes an Ad35 fiber knob.

26. The genome, vector, system, or method of claim 25, wherein the Ad35 fiber knob comprises a mutation that increases affinity with CD46.

27. The helper genome, helper vector, system, or method of claim 25 or claim 26, wherein the Ad35 fiber knob comprises one or more mutations:
selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or
comprising each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

28. The helper genome, helper vector, system, or method of any one of claims 1-27, wherein the helper genome is present in a cell that comprises a nucleic acid encoding a recombinase for recombination of the direct repeats.

29. The helper genome, helper vector, system, or method of claim 28, wherein the recombinase is a Flp, Cre, Dre, Vika, or PhiC31 recombinase.

30. The helper genome, helper vector, system, or method of claim 28 or claim 29, wherein the cell is a HEK293 cell, optionally wherein the cell is a HEK293 cell that encodes or expresses Cre recombinase, optionally wherein the HEK293 cell that encodes or expresses Cre recombinase is a 116 cell.

31. The helper genome, helper vector, system, or method of any one of claims 1-30, wherein the helper genome comprises an inverted packaging sequence.

32. A recombinant adenoviral helper genome comprising:
a 5′ inverted terminal repeat (ITR);
a 3′ ITR; and
a packaging sequence;
wherein the 5′ ITR, the 3′ ITR, and the packaging sequence are each derived from a species B adenovirus of a serotype selected from Ad3, Ad7, Ad11, Ad14, Ad16, Ad21, Ad34, or Ad50;
wherein the packaging sequence is flanked by or comprises recombinase direct repeats comprising a first recombinase direct repeat and a second recombinase direct repeat;
wherein the position of the first recombinase direct repeat corresponds to a position that is within 10 nucleotides of an L1, L2, L3, or L4 site of a reference sequence for the serotype of the packaging sequence, as set forth in Table 21; and
wherein the position of the second recombinase direct repeat corresponds to a position that is within 10 nucleotides of an R1, R2, or R3 site of a reference sequence for the serotype of the packaging sequence, as set forth in Table 21; and
wherein the helper genome comprises an inverted packaging sequence.

33. The helper genome of claim 32, wherein the 5′ ITR and the 3′ ITR are derived from the same serotype.

34. The helper genome of claim 32 or claim 33, wherein the 5′ ITR, the 3′ ITR, and the packaging sequence are derived from the same serotype.

35. The helper genome of any one of claims 32-34, wherein the recombinase direct repeats that flank the packaging sequence are FRT, loxP, rox, vox, AttB, or AttP sites.

36. The helper genome of any one of claims 32-35, wherein the recombinase direct repeats that flank the packaging sequence are loxP sites.

37. A recombinant adenoviral helper vector comprising the helper genome of any one of claims 32-36.

38. A recombinant adenoviral vector production system comprising:
(i) the helper genome of any one of claims 32-36 or the helper vector of claim 37, and
(ii) a helper-dependent adenoviral (HDAd) donor genome, the HDAd donor genome comprising:
a 5′ inverted terminal repeat (ITR);
a 3′ ITR;
a packaging sequence; and
a nucleic acid sequence encoding at least one heterologous expression product;
wherein the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50.

39. A method of producing a recombinant helper-dependent adenoviral (HDAd) donor vector, the method comprising isolating the recombinant HDAd donor vector from a culture of cells, wherein the cells comprise:
a recombinant helper genome of any one of claims 32-36 or a recombinant adenoviral helper vector of claim 37; and
a recombinant HDAd donor genome comprising:
a 5′ inverted terminal repeat (ITR);
a 3′ ITR;
an packaging sequence; and
a nucleic acid sequence encoding at least one heterologous expression product;
wherein the 5′ ITR, the 3′ ITR, and the packaging sequence of the HDAd donor genome are each derived from a species B adenovirus of a serotype selected from Ad3, Ad7, Ad11, Ad14, Ad16, Ad21, Ad34, or Ad50.

40. The system or method of claim 38 or claim 39, wherein the 5′ ITR and the 3′ ITR of the HDAd donor genome are derived from the same serotype.

41. The system or method of any one of claims 38-40, wherein the 5′ ITR, the 3′ ITR, and the packaging sequence of the HDAd donor genome are derived from the same serotype.

42. The helper genome, helper vector, system, or method of any one of claims 32-41, wherein the helper genome comprises a nucleic acid sequence that encodes an Ad35 fiber knob.

43. The genome, vector, system, or method of claim 42, wherein the Ad35 fiber knob comprises a mutation that increases affinity with CD46.

44. The helper genome, helper vector, system, or method of claim 42 or claim 43, wherein the Ad35 fiber knob comprises one or more mutations:
selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or
comprising each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

45. The helper genome, helper vector, system, or method of any one of claims 32-44, wherein the helper genome is present in a cell that comprises a nucleic acid encoding a recombinase for recombination of the direct repeats.

46. The helper genome, helper vector, system, or method of claim 45, wherein the recombinase is a Flp, Cre, Dre, Vika, or PhiC31 recombinase.

47. The helper genome, helper vector, system, or method of claim 45 or claim 46, wherein the cell is a HEK293 cell, optionally wherein the cell is a HEK293 cell that encodes or expresses Cre recombinase, optionally wherein the HEK293 cell that encodes or expresses Cre recombinase is a 116 cell.

48. The helper genome, helper vector, system, or method of any one of claims 31-47, wherein the inverted packaging sequence comprises the packaging sequence and one or both of the first recombinase direct repeat and the second recombinase direct repeat.

49. The helper genome, helper vector, system, or method of any one of claims 31-48, wherein the inverted packaging sequence comprises, or comprises a first end point at, a nucleotide position corresponding to a position that is within 25 nucleotides of a Left Inversion Point of a reference sequence for the serotype of the packaging sequence (e.g., at the Left Inversion Point, no more than 25 nucleotides 5′ of the Left Inversion Point, and/or no more than 25 nucleotides 3′ of the Left Inversion Point), as set forth in Table 28.

50. The helper genome, helper vector, system, or method of any one of claims 31-48, wherein the inverted packaging sequence comprises, or comprises a first end point at, a nucleotide position corresponding to a position that is within 10 nucleotides of a Left Inversion Point of a reference sequence for the serotype of the packaging sequence, as set forth in Table 28.

51. The helper genome, helper vector, system, or method of any one of claims 31-48, wherein the inverted packaging sequence comprises, or comprises a first end point at, a nucleotide position corresponding to a position at a Left Inversion Point of a reference sequence for the serotype of the packaging sequence, as set forth in Table 28.

52. The helper genome, helper vector, system, or method of any one of claims 31-48, wherein the inverted packaging sequence comprises, or comprises a first end point at, a nucleotide position corresponding to a position at a Left Inversion Point of a reference sequence for the serotype of the packaging sequence, as set forth in any one of Tables 29-35.

53. The helper genome, helper vector, system, or method of any one of claims 31-52, wherein the inverted packaging sequence comprises, or comprises a second end point at, a nucleotide position corresponding to a position that is within 25 nucleotides of a Right Inversion Point of a reference sequence for the serotype of the packaging sequence (e.g., at the Right Inversion Point, no more than 25 nucleotides 5′ of the Right Inversion Point, and/or no more than 25 nucleotides 3′ of the Right Inversion Point), as set forth in Table 28.

54. The helper genome, helper vector, system, or method of any one of claims 31-52, wherein the inverted packaging sequence comprises, or comprises a second end point at, a nucleotide position corresponding to a position that is within 10 nucleotides of a Right Inversion Point of a reference sequence for the serotype of the packaging sequence, as set forth in Table 28.

55. The helper genome, helper vector, system, or method of any one of claims 31-52, wherein the inverted packaging sequence comprises, or comprises a second end point at, a nucleotide position corresponding to a position at a Right Inversion Point of a reference sequence for the serotype of the packaging sequence, as set forth in Table 28.

56. The helper genome, helper vector, system, or method of any one of claims 31-52, wherein the inverted packaging sequence comprises, or comprises a second end point at, a nucleotide position corresponding to a position at a Right Inversion Point of a reference sequence for the serotype of the packaging sequence, as set forth in any one of Tables 29-35.

57. A recombinant recombinase site-flanked adenoviral packaging sequence, wherein recombinase direct repeats flank a packaging sequence, and wherein the packaging sequence is derived from a species B adenovirus of a serotype selected from Ad3, Ad7, Ad11, Ad14, Ad16, Ad21, Ad34, or Ad50; and wherein the packaging sequence corresponds to a fragment of an adenoviral genome having:
(i) a first end point that corresponds to a position that is within 10 nucleotides of an L1, L2, L3, or L4 site of a reference sequence for the serotype of the packaging sequence, as set forth in Table 21, and
(ii) a second end point that corresponds to a position that is within 10 nucleotides of an R1, R2, or R3 site of a reference sequence for the serotype of the packaging sequence, as set forth in Table 21.

58. The recombinant packaging sequence of claim 57, wherein the first end point corresponds to a position that is within 10 nucleotides of an L1 site of a reference sequence for the serotype of the packaging sequence, as set forth in Table 21; and wherein the second end point corresponds to a position that is within 10 nucleotides of an R3 site of a reference sequence for the serotype of the packaging sequence, as set forth in Table 21.

59. The recombinant packaging sequence of claim 57, wherein the first end point corresponds to a position that is within 10 nucleotides of an L2 site of a reference sequence for the serotype of the packaging sequence, as set forth in Table 21; and wherein the second end point corresponds to a position that is within 10 nucleotides of an RI site of a reference sequence for the serotype of the packaging sequence, as set forth in Table 21.

60. The recombinant packaging sequence of claim 57, wherein the first end point corresponds to a position that is within 10 nucleotides of an L3 site of a reference sequence for the serotype of the packaging sequence, as set forth in Table 21; and wherein the second end point corresponds to a position that is within 10 nucleotides of an R2 site of a reference sequence for the serotype of the packaging sequence, as set forth in Table 21.

61. The recombinant packaging sequence of claim 57, wherein the first end point corresponds to a position that is within 10 nucleotides of an L4 site of a reference sequence for the serotype of the packaging sequence, as set forth in Table 21; and wherein the second end point corresponds to a position that is within 10 nucleotides of an RI site of a reference sequence for the serotype of the packaging sequence, as set forth in Table 21.

62. The recombinant packaging sequence of claim 57, wherein the first end point corresponds to a position at an L1, L2, L3, or L4 site of a reference sequence for the serotype of the packaging sequence, as set forth in Table 21; and wherein the second end point corresponds to a position at an R1, R2, or R3 site of a reference sequence for the serotype of the packaging sequence, as set forth in Table 21.

63. The recombinant packaging sequence of claim 57, wherein the first end point corresponds to a position at an L1 site of a reference sequence for the serotype of the packaging sequence, as set forth in Table 21; and wherein the second end point corresponds to a position at an R3 site of a reference sequence for the serotype of the packaging sequence, as set forth in Table 21.

64. The recombinant packaging sequence of claim 57, wherein the first end point corresponds to a position at an L2 site of a reference sequence for the serotype of the packaging sequence, as set forth in Table 21; and wherein the second end point corresponds to a position at an R1 site of a reference sequence for the serotype of the packaging sequence, as set forth in Table 21.

65. The recombinant packaging sequence of claim 57, wherein the first end point corresponds to a position at an L3 site of a reference sequence for the serotype of the packaging sequence, as set forth in Table 21; and wherein the second end point corresponds to a position at an R2 site of a reference sequence for the serotype of the packaging sequence, as set forth in Table 21.

66. The recombinant packaging sequence of claim 57, wherein the first end point corresponds to a position at an L4 site of a reference sequence for the serotype of the packaging sequence, as set forth in Table 21; and wherein the second end point corresponds to a position at an R1 site of a reference sequence for the serotype of the packaging sequence, as set forth in Table 21.

67. The recombinant packaging sequence of claim 57, wherein the first end point corresponds to a position at an L1, L2, L3, or L4 site of a reference sequence for the serotype of the packaging sequence, as set forth in any one of Tables 22-27; and wherein the second end point corresponds to a position at an R1, R2, or R3 site of a reference sequence for the serotype of the packaging sequence, as set forth in any one of Tables 22-27.

68. The recombinant packaging sequence of claim 57, wherein the first end point corresponds to a position at an L1 site of a reference sequence for the serotype of the packaging sequence, as set forth in any one of Tables 22-27; and wherein the second end point corresponds to a position at an R3 site of a reference sequence for the serotype of the packaging sequence, as set forth in any one of Tables 22-27.

69. The recombinant packaging sequence of claim 57, wherein the first end point corresponds to a position at an L2 site of a reference sequence for the serotype of the packaging sequence, as set forth in any one of Tables 22-27; and wherein the second end point corresponds to a position at an R1 site of a reference sequence for the serotype of the packaging sequence, as set forth in any one of Tables 22-27.

70. The recombinant packaging sequence of claim 57, wherein the first end point corresponds to a position at an L3 site of a reference sequence for the serotype of the packaging sequence, as set forth in any one of Tables 22-27; and wherein the second end point corresponds to a position at an R2 site of a reference sequence for the serotype of the packaging sequence, as set forth in any one of Tables 22-27.

71. The recombinant packaging sequence of claim 57, wherein the first end point corresponds to a position at an L4 site of a reference sequence for the serotype of the packaging sequence, as set forth in any one of Tables 22-27; and wherein the second end point corresponds to a position at an R1 site of a reference sequence for the serotype of the packaging sequence, as set forth in any one of Tables 22-27.

72. The recombinant packaging sequence of any one of claims 57-71, wherein the packaging sequence is present in an adenoviral genome and is inverted, optionally wherein the packaging sequence is inverted as compared to a 5′ITR of the adenoviral genome.

73. A recombinant adenoviral helper genome comprising:
a 5′ inverted terminal repeat (ITR);
a 3′ ITR; and
an inverted sequence comprising a packaging sequence;
wherein the 5′ ITR, the 3′ ITR, and the packaging sequence are each derived from a species B adenovirus of a serotype selected from Ad3, Ad7, Ad11, Ad14, Ad16, Ad21, Ad34, or Ad50; and
wherein the inverted sequence comprises, or comprises a first end point at, a nucleotide position corresponding to a position within 25 nucleotides of a Left Inversion Point (e.g., within 10 nucleotides of a Left Inversion Point, e.g., at a Left Inversion Point) of a reference sequence for the serotype of the packaging sequence, as set forth in Table 28; and
wherein the inverted sequence comprises, or comprises a second end point at, a nucleotide position corresponding to a position within 25 nucleotides of a Right Inversion Point (e.g., within 10 nucleotides of a Right Inversion Point, e.g., at a Right Inversion Point) of a reference sequence for the serotype of the packaging sequence, as set forth in Table 28.

74. The recombinant adenoviral helper genome of claim 73, wherein the 5′ ITR and the 3′ ITR are derived from the same serotype.

75. The recombinant adenoviral helper genome of claim 73 or claim 74, wherein the 5′ ITR, the 3′ ITR, and the packaging sequence are derived from the same serotype.

76. The recombinant adenoviral helper genome of any one of claims 73-75, wherein recombinase direct repeats flank the packaging sequence.
